(19) United States
(12) Patent Application Publication
(10) Pub. No.: US 2022/0106640 A1
(43) Pub. Date: Apr. 7, 2022

(54) METHODS OF DETECTING DNA AND RNA IN THE SAME SAMPLE

(71) Applicant: HTG Molecular Diagnostics, Inc., Tucson, AZ (US)

(72) Inventors: Debrah Thompson, Tucson, AZ (US); Matthew Rounseville, Tucson, AZ (US)

(73) Assignee: HTG Molecular Diagnostics, Inc., Tucson, AZ (US)

(21) Appl. No.: 17/416,982

(22) PCT Filed: Dec. 2, 2019
(86) PCT No.: PCT/US2019/064041

§ 371 Date: Jun. 21, 2021

Related U.S. Application Data
(60) Provisional application No. 62/787,114, filed on Dec. 31, 2018.

(51) Int. Cl. C12Q 1/6874; C12Q 1/6853

(52) U.S. Cl. CPC C12Q 1/6874; C12Q 2600/16; C12Q 1/6853

(57) ABSTRACT

The present disclosure provides methods for sequencing nucleic acid targets (e.g., both DNA and RNA co-amplified in a sample mixture, for example by using a surrogate for the RNA). Such methods can be used to determine if one or more nucleic acid targets are present in a sample.

### CROSS-REFERENCE TO RELATED APPLICATION

This application claims priority to U.S. Provisional Application No. 62/787,114 filed Dec. 31, 2018, herein incorporated by reference in its entirety.

### FIELD

The present disclosure provides quantitative nuclease protection sequencing (qNPS) methods that allow sequencing of nucleic acid targets (for example by co-amplifying DNA and an RNA surrogate in the same sample). Such methods can be used to determine if one or more nucleic acid targets are present in a sample, and in some examples is quantitative.

### BACKGROUND

Although methods of sequencing nucleic acid molecules are known, there is still a need for methods that permit sequencing of RNA and DNA co-amplified in the sample mixture. Methods of multiplexing nucleic acid molecule sequencing reactions that utilize DNA and RNA co-amplified in the sample mixture have not been realized at the most desired performance or simplicity levels.

### SUMMARY

Methods are provided that improve prior quantitative nuclease protection sequencing (qNPS) methods (such as those disclosed in U.S. Publication No. US 2011-0104693 and U.S. Pat. No. 8,741,564) and represent an improvement to current nucleic acid sequencing methods. In some examples, the disclosed methods sequence or detect at least one target DNA and at least one target RNA in the same sample (such as the same biopsy sample or the same tissue sample), by co-amplifying both molecules from the same sample, by use of an RNA surrogate molecule. In some examples, a plurality of different (e.g., unique) samples are analyzed simultaneously. In some examples, the target RNA and DNA molecules have a point mutation, a deletion, insertion, or combinations thereof. In some examples, the method determines the abundance (e.g., quantitatively or qualitatively) of one or more target RNAs and determines if genomic mutations are present in one or more target DNA sequences.

The disclosed methods of determining a sequence of a target DNA molecule (e.g., a target genomic molecule) and a target RNA molecule (e.g., a target mRNA or target miRNA molecule) in a sample (e.g., a fixed sample, such as a formalin-fixed sample) can include lysing the sample with a lysis buffer (e.g., a lysis buffer that includes a detergent and/or a chaotropic agent), thereby generating a lysate comprising the target DNA molecule and the target RNA molecule. The lysate is divided into at least two different portions, for example of equal volume or of equal nucleic acid content.

The target DNA is amplified from a first portion of the lysate using at least one primer (e.g., a target DNA primer, such as a first forward primer and first reverse primer), thereby generating flanked amplicon regions (FARs). In some examples, amplifying the target DNA from the first portion of the cell lysate uses at least two primers (e.g., at least two target DNA primers), each DNA primer having a flanking sequence at its 5′ end. For example, the first target DNA primer (such as a forward primer) can have at its 5′-end a flanking sequence that is the reverse-complement sequence of the 3′-flanking sequence of the nuclease protection probe that includes a flanking sequence (NPPF—see below), while the second target DNA primer (such as a reverse primer) can have at its 5′-end a flanking sequence identical to the 5′-flanking sequence of the NPPF. These flanking sequences on the DNA primers allow flanking sequences to be added to the DNA amplicons, thereby generating flanked amplicon regions (FARs). In some examples, the flanking sequences added are about 10 to 50 nucleotides (nt) each, such as 25 nt each. In some examples, the DNA amplified from the target is about 40 to 150 nt in length, such as 40 to 125 nt or 40 to 100 nt. In some examples, the FAR generated is about 100 to 200 nt in length, such as 160 to 200 nt.

A second (i.e., different) portion of the lysate is incubated with at least one nuclease protection probe that includes a flanking sequence (NPPF) under conditions sufficient for the NPPF to specifically bind to the target RNA molecule present in the second portion of the lysate. In some examples the NPPF is a DNA molecule about 50 to 200 nt in length, such as 60 to 200 nt, 75 to 150, or 65 to 100 nt. The NPPF includes (1) a 5′-end, (2) a 3′-end, (3) a sequence (e.g., about 10-60 nt in length, such as 16 to 50 nt) that is complementary to all or a portion of the target RNA molecule, thus permitting specific binding or hybridization between the target RNA molecule and the NPPF, and (4) a flanking sequence. For example, the region of the NPPF that is complementary to a region of the target RNA molecule binds to or hybridizes to that region of the target RNA molecule with high specificity. In some examples, the flanking sequence is located 5′, 3′, or both to the sequence complementary to the target RNA molecule, such as a 5′-flanking sequence 5′ of the sequence complementary to the target RNA molecule and a 3′-flanking sequence 3′ of the sequence complementary to the target RNA molecule. In some examples, the flanking sequence includes at least 12 contiguous nucleotides not found in a nucleic acid molecule present in the sample.

In some examples, the NPPF includes a 5′-flanking sequence, and the methods further include contacting the second portion of the lysate with a nucleic acid molecule (e.g., DNA or RNA) that includes a sequence complementary to the 5′-flanking sequence (5CFS) under conditions sufficient for the 5′-flanking sequence to specifically hybridize to the 5CFS. In some examples, the NPPF includes a 3′-flanking sequence, and the method further includes contacting the second portion of the lysate with a nucleic acid molecule (e.g., DNA or RNA) that includes a sequence complementary to the 3′-flanking sequence (3CFS) under conditions sufficient for the 3′-flanking sequence to specifically hybridize to the 3CFS. In some examples, the NPPF includes a 3′- and a 5′-flanking sequence, and the method further includes contacting the second portion of the lysate with a 3CFS and 5CFS under conditions sufficient for the 3′-flanking sequence to specifically hybridize to the 3CFS and the 5′-flanking sequence to specifically hybridize to the 5CFS. Hybridization results in the generation of a double-stranded (ds) nucleic acid molecule, namely NPPF hybridized to (1) the target RNA molecule, and (2) the 5CFS and/or 3CFS. In some examples, at least one nucleotide in the NPPF does not have complementarity to the corresponding nucleotide in the target RNA molecule or does not have complementarity to the corresponding nucleotide in the 5CFS or 3CFS.

The resulting double-stranded (ds) nucleic acid molecule, namely NPPF hybridized to (1) the target RNA molecule, and (2) the 5CFS and/or 3CFS present in the second portion of the lysate is contacted with a nuclease specific for single-stranded (ss) nucleic acid molecules (e.g., an exonuclease, an endonuclease, or a combination thereof, such as S1 nuclease) under conditions sufficient to degrade (hydrolyze) or remove unbound ss nucleic acid molecules in the second portion of the lysate. Thus for example, NPPFs that have not bound target RNA or CFSs, unbound RNA molecules, unbound portions of target RNA molecules, unbound CFSs, and other ss nucleic acid molecules in the second portion of the lysate, are degraded. This results in a second portion of the lysate containing a digested sample that includes an NPPF hybridized to its target RNA molecule, hybridized to its corresponding 3CFS, hybridized to its corresponding 5CFS, or hybridized to both its corresponding 3CFS and its corresponding 5CFS.

This ds nucleic acid molecule (NPPF: target RNA molecule:CFS) in the second portion of the lysate can be separated into its corresponding ss nucleic acid molecules (for example by heating, for example heating to 95° C. to 100° C.), thereby generating a mixture of ssNPPFs, ssCFSs, and ss target RNA molecules. In some examples, this separation occurs as the first step of the second amplification (amplification of the FARs and ssNPPFs) described below. In one example, the RNA strand of the NPPF:RNA target can be selectively removed by treating the complex with RNase H, which selectively removes the RNA moiety of a DNA:RNA complex (for example, if the if the target molecule is RNA, the NPPF is DNA, and the 3CFS and 5 CFS are DNA). Alternative nucleases can be used to optionally degrade RNA separately from DNA.

The methods include mixing or combining the FARs generated in the first portion of the sample lysate with the second portion of the sample lysate containing the ssNPPFs, thereby generating a DNA amplicons/ssNPPF mixture. In some examples, the first portion of the cell lysate containing the DNA amplicons is added to the second portion of the cell lysate containing the ssNPPFs (or vice versa). In some examples, a 1:1, 1:2, 1:3, 1:4, 1:5, or 1:10 ratio of ssNPPFs:FARs is used in the subsequent amplification step.

The resulting FARs/ssNPPF mixture is incubated with appropriate primers (such as forward and reverse primers), under conditions that co-amplify the FARs and the ssNPPFs in the same reaction vessel (e.g., same microfuge tube or same well of a multi-well plate). In some examples, different primers are used to amplify the FARs, and to amplify the ssNPPFs. In some examples the same forward and reverse primers are used to amplify the FARs, and to amplify the ss NPPFs, for example due to the presence of identical 5′- and 3-flanking sequences on the FARs and the ssNPPFs (e.g., the NPPF includes a 5′-flanking sequence and a 3′-flanking sequence, and the FARs include the same 5′-flanking sequence and same 3′-flanking sequence as that in the NPPF). For example, the amplification can use a first amplification primer having a region identical to the 5′-flanking sequence and a second amplification primer having a region complementary to the 3′-flanking sequence. Such primers can further include one or more sequences that permit attachment of an experimental tag, sequencing adaptor, or both, to the FAR amplicons or NPPF amplicons (for example to the 5′-end, 3′-end, or both of the resulting amplicons) during the amplification of the FARs and the single stranded NPPFs. In some examples, the methods further include removing the amplification primers after amplifying the FARs and the ssNPPFs but before sequencing the FAR amplicons and the NPPF amplicons.

In some examples, the NPPF includes both a 5′-flanking sequence and a 3′-flanking sequence (such as a flanking sequence at the 5′-end that differs from the flanking sequence at the 3′-end), and the FARs include the same 5′-flanking sequence and same 3′-flanking sequence as those in the NPPF. Thus, after separating the ds NPPF:RNA target:CFS molecule into a ss NPPF molecule, but before sequencing, the methods can include contacting the ssNPPF (and in some examples also the FAR with the same 5′- and 3′-end flanking sequences) with a first amplification primer that includes a region complementary to the 3′-flanking sequence and with a second amplification primer that includes a region complementary to the 5′-flanking sequence. For example, the first and second amplification primers can permit attachment of an experimental tag (e.g., a nucleic acid sequence that permits identification of a sample, subject, treatment or target RNA or DNA molecule) and/or sequencing adaptor (e.g., a nucleic acid sequence that permits capture onto a sequencing platform) to the resulting NPPF amplicons (and FAR amplicons) (such as an experiment tag or sequence adaptor on the 5′-end or 3′-end of the NPPF amplicons and FAR amplicons), such as a first amplification primer that permits attachment of a first experimental tag and/or first sequencing adaptor to the NPPF amplicons and FAR amplicons and a second amplification primer that permits attachment of a second experimental tag and/or second sequencing adaptor to the NPPF amplicons and FAR amplicons. In some examples, the methods further include removing the first and second amplification primers after amplifying but before sequencing (such as removing amplification primers after the amplifying the target DNA from a first portion of the lysate using at least one target DNA primer, removing the first and second amplification primers after the amplifying of the FARs and the single stranded NPPF, or removing both sets of amplification primers, before the sequencing step).

The methods can further include sequencing (e.g., next generation sequencing or single molecule sequencing) at least a portion of the resulting NPPF amplicons and at least a portion of the FAR amplicons, thereby determining the sequence of the target DNA molecule (via the FAR amplicons), the sequence of (and/or abundance of) the target RNA molecule (via the NPPF amplicons) in the sample.

In some examples, the methods sequence or detect at least two different target RNA molecules (e.g., where the sample is contacted with at least two different NPPFs, such as where each NPPF is specific for a different target RNA molecule, or where the sample is contacted with at least one NPPF specific for the at least two different target RNA molecules, such as separate RNA molecules transcribed from different loci, or more than one alternative transcript or splice isoform transcribed from the same locus). In some examples, the methods sequence or detect at least two different target DNA molecules (e.g., where the at least two different target DNA molecules include a wild type gene sequence and at least one mutation in the gene sequence). In specific examples, the methods can be performed on a plurality of samples with, for example, at least two different target RNA molecules and at least two different target DNA molecules detected in each of the plurality of samples. In specific examples, at least one NPPF is specific for a miRNA target nucleic acid molecule and at least one NPPF is specific for an mRNA target nucleic acid molecule.

Also provided are isolated nucleic acid molecules, such as one comprising or consisting of the nucleic acid sequence of any one of SEQ ID NO: 4, 5, 6, 7, 8, 9, 10, 11, 12, 13, 17, 18, 19, 20, 21, 22, 23, 24, 25, 26, 27, 28, 29, 30, 31 or 32. Also provided are sets of nucleic acid primers, for example as part of a kit. In some examples, the set includes the nucleic acid sequence of SEQ ID NOs: 4 and 5; SEQ ID NOs: 6 and 7; SEQ ID NOs: 8 and 9; SEQ ID NOs: 10 and 11; SEQ ID NOs: 12 and 13; SEQ ID NOs: 17 and 18; SEQ ID NOs: 19 and 20; SEQ ID NOs: 21 and 22; SEQ ID NOs: 23 and 24; SEQ ID NOs: 25 and 26; SEQ ID NOs: 27 and 28; SEQ ID NOs: 29 and 30; SEQ ID NOs: 31 and 32; or combinations of these sets (such as at least two or at least three of these sets).

The foregoing and other objects and features of the disclosure will become more apparent from the following detailed description, which proceeds with reference to the accompanying figures.

### BRIEF DESCRIPTION OF THE DRAWINGS

FIG. 1A is a schematic diagram showing an exemplary nuclease protection probe having flanking sequences (NPPF), 100. The NPPF 100 includes a region 102 having a sequence that specifically binds to/hybridizes to a target nucleic acid sequence (e.g., target RNA sequence). The NPPF also includes a 5′-flanking sequence 104, a 3′-flanking sequence 106, or both (the embodiment with both is shown).

FIG. 1B is a schematic diagram showing an exemplary nuclease protection probe having flanking sequences (NPPF), 120. In this example, the NPPF 120 is composed of two separate nucleic acid molecules 128, 130, instead of a single nucleic acid molecule as shown in FIG. 1A. The NPPF 120 includes a region 122 having a sequence that specifically binds to/hybridizes to a target nucleic acid sequence. The NPPF also includes a 5′-flanking sequence 124, a 3′-flanking sequence 126, or both (the embodiment with both is shown).

FIG. 2 is a schematic diagram showing an overview of the steps of an illustrative method for lysing the sample 10, dividing the lysed sample into at least two portions, wherein target DNA is amplified in a first portion 12, generating FARs specific for the target DNA, and target RNA is hybridized to NPPFs, nuclease digested, and ds nucleic acid molecules denatured, generating ssNPPFs specific for the target RNA in second portion 14, then at least a portion of first and second portions combined and the FARs and NPPFs co-amplified in the mixture 16, prior to sequencing and data extraction 18.

FIG. 3 is a schematic diagram showing an overview of the steps of an illustrative method for sequencing of at least one target DNA molecule and at least one target RNA molecule, wherein the DNA and a surrogate of the RNA are amplified in the sample mixture. Step 1 shows a sample (such as cells or FFPE tissue), which is contacted with sample disruption buffer (for example to permit lysis of cells and tissues in the sample) and then separated into at least two portions (first and second portion). Step 2A shows that a first portion of the cell lysate is incubated with two amplification primers (e.g., target DNA primers), such as a first primer containing a 5′ extension 234 and a second primer containing a 5′ extension 232 under conditions that allow for hybridization of the primers to the target DNA 230. Step 2B shows that the target DNA molecule 230 is amplified using the primers 234, 232, generating a flanked amplicon region (FAR) 236 with 5′ and 3′ extensions from the primers (in some examples the 5′- and 3′-extensions of the FAR (shown as 238, 239, respectively) are identical to the 5′- and 3′-flanking sequences of the NPPF (204, 206). Step 2AA shows that a second portion of the cell lysate is incubated with at least one NPPF 202 and its complementary 5CFS 208 and 3CFS 210 under conditions that allow specific hybridization of the NPPF 202 to a target RNA 200, and to the CFSs 208, 210. Step 2BB shows that the resulting ds nucleic acid molecule generated in Step 2AA, is incubated with a nuclease specific for ss nucleic acid molecules (such as S1 nuclease, mung bean nuclease, BAL 31 nuclease, or P1 nuclease), resulting in a ds NPPF/RNA/CFSs target complex 212. Step 2CC shows that the ds NPPF/RNA target complex 212 is then separated or denature into its single nucleic acid strands, generating a mixture of ssRNA 200, ss CFSs 208, 210, and ssNPPF 202. In Step 3, the mixture of ssRNA 200, ss CFSs 208, 210 and ssNPPF 202 is combined with the DNA amplicons 236. In Step 4, the combined ssNPPF 202 and FARs 236 are co-amplified in the same reaction, for example, by using PCR with appropriate primers, and then sequenced.

FIG. 4 is a schematic diagram showing amplification of ssNPPF 200 (RNA target surrogate) and FAR 236 using forward and reverse primers (arrows), resulting in NPPF amplicons 226 and FAR amplicons 246, respectively. The primers can include sequences that allow sequencing adaptors 218, 220, 248, 240 and/or experiment tags 222, 224, 242, 244 to be added to the NPPF amplicons 226 and FAR amplicons 246, respectively. The resulting NPPF amplicons 226 are used to detect target RNA (and can be used to determine a target RNA sequence and/or its abundance), and FAR amplicons 246 are used to detect target DNA (and can be used to determine a target DNA sequence). In some examples, the primer sequences are used to identify amplicons (such as NPPF amplicons 226 and FAR amplicons 246) as a product of the same sample, in which, some examples of the methods include primers where the adaptor and/or tag sequences are the same (e.g., in such examples, sequences 218, 222 are the same as 248, 242, and sequences 224, 220 are the same as 244, 240).

FIGS. 5A-5B show scatterplots with Pearson correlations for raw data from triplicate experiments for a formalin-fixed, paraffin-embedded (FFPE) sample (FIG. 5A) and a cell line mixture sample (FIG. 5B).

FIG. 6 shows expression of the indicated RNA measured in a cell line titration series from triplicate experiments.

FIG. 7 shows DNA mutations detected in cell line samples as a percentage of the total counts for the indicated region (BRAF left, KRAS right) from triplicate experiments performed on three different days.

FIG. 8 shows the average of raw counts in cell line titration from triplicate experiments performed on three different days (BRAF V600E left, KRAS G12D right) FIG. 9 shows the percentage of total reads consumed by NPPFs/RNA (grey) and by FARs/DNA (hatched grey) for one sample under the different conditions used.

FIG. 10 shows the results for a single set of conditions (14 cycles and 4 ul added) for all seven FFPE samples. The graph shows the percentage of total reads consumed by NPPFs or RNA (grey) and by FARs or DNA (hatched grey).

FIG. 11 shows DNA mutation information and BRAF mutation detection in eight FFPE samples as a percentage of total BRAF signal (SEQ ID NOS: 14-16, from top to bottom).

FIGS. 12A-12B show scatterplots of RNA expression data generated using a set of 470 NPPS for two of the eight FFPE samples (FFPE1 (lung, FIG. 12A) and FFPE7591 (melanoma, FIG. 12B)). Pearson correlations (r) for triplicate measurements are displayed on the scatterplots.

FIG. 13 shows a principal component analysis (PCA) plot of RNA expression data from nine replicates of samples from cell lines HD300, HD301, and HD789. The three different cell lines are strongly separated, demonstrating the differences in expression profiles. The replicates are tightly clustered together, demonstrating excellent repeatability between technical replicates and replicates run on different days.

FIG. 14 is a table showing observed and expected allelic frequencies for each of the three reference standards and the three mixture samples.

FIG. 15 shows a bar graph and table demonstrating the repeatability of individual measurements of DNA variants.

### SEQUENCE LISTING

The nucleic acid and protein sequences are shown using standard letter abbreviations for nucleotide bases as defined in 37 C.F.R. 1.822. Only one strand of each nucleic acid sequence is shown, but the complementary strand is understood as included by any reference to the displayed strand. The contents of the text file named “seq listing”, which was created on Dec. 2, 2019 and is about 4 KB in size, are hereby incorporated by reference in their entirety.

SEQ ID NO: 1 shows an exemplary 5′-flanking sequence.

SEQ ID NO: 2 shows an exemplary 3′-flanking sequence.

SEQ ID NO: 3 shows an exemplary reverse-complement of a 3′-flanking sequence.

SEQ ID NOs: 4 and 5 show exemplary forward and reverse primers, respectively, for amplifying BRAF.

SEQ ID NOs: 6 and 7 show exemplary forward and reverse primers, respectively, for amplifying KRAS.

SEQ ID NOs: 8 and 9 show exemplary forward and reverse primers, respectively, for amplifying EGFR.

SEQ ID NOs: 10 and 11 show exemplary forward and reverse primers, respectively, for amplifying EGFR.

SEQ ID NOs: 12 and 13 show exemplary primers that can be used to add an experiment tag to the resulting amplicon.

SEQ ID NOs: 14-16 show three BRAF sequences: Wild type, nt mutation giving rise to V600E mutation, and another nt mutation giving rise to V600E2 mutation.

SEQ ID NOs: 17 and 18 show exemplary forward and reverse primers, respectively, for amplifying BRAF to detect a V600 mutation.

SEQ ID NOs: 19 and 20 show exemplary forward and reverse primers, respectively, for amplifying EGFR to detect a G719 mutation.

SEQ ID NOs: 21 and 22 show exemplary forward and reverse primers, respectively, for amplifying EGFR to detect mutations within exon 19.

SEQ ID NOs: 23 and 24 show exemplary forward and reverse primers, respectively, for amplifying EGFR to detect mutations within exon 20.

SEQ ID NOs: 25 and 26 show exemplary forward and reverse primers, respectively, for amplifying EGFR to detect a L858F or L858-L861 mutation.

SEQ ID NOs: 27 and 28 show exemplary forward and reverse primers, respectively, for amplifying KRAS to detect a G12 mutation.

SEQ ID NOs: 29 and 30 show exemplary forward and reverse primers, respectively, for amplifying KRAS to detect a Q61 mutation.

SEQ ID NOs: 31 and 32 show exemplary forward and reverse primers, respectively, for amplifying PIK3CA.

### DETAILED DESCRIPTION

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, and Proteomics, 2nd Edition, 2003 (ISBN: 0-471-26821-6).

The singular forms “a,” “an,” and “the” refer to one or more than one, unless the context clearly dictates otherwise. For example, the term “comprising an NPPF” includes single or plural NPPFs and is considered equivalent to the phrase “comprising at least one NPPF.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety, as are the GenBank® Accession numbers (for the sequence present on Dec. 31, 2018). In case of conflict, the present specification, including explanations of terms, will control. In addition, the materials, methods, and examples are illustrative only and not intended to be limiting.

Except as otherwise noted, the methods and techniques of the present disclosure are generally performed according to conventional methods well known in the art and as described in various general and more specific references that are cited and discussed throughout the present specification. See, e.g., Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

### I. Overview

The present disclosure provides methods that allow for sequencing of target nucleic acid molecules, such as target DNA and target RNA (using a NPPF surrogate) co-amplified in the sample mixture, which methods further can be multiplexed (e.g., detecting a plurality of DNA and RNA targets in a single sample) or are amenable to high-throughput (e.g., detecting DNA and RNA targets in a plurality of samples, e.g., different samples) or are multiplexed and high-throughput (e.g., detecting a plurality of DNA and RNA targets in a plurality of sample, e.g., different samples). The disclosed methods provide several improvements over currently available sequencing methods. For example, because the methods co-amplify target DNA (generating amplicons referred to herein as FAR amplicons) and NPPFs (generating NPPF amplicons, which serve as surrogates of target RNA) in the same reaction vessel, these allow for analysis of DNA and RNA from the same sample, instead of from two different samples (i.e., one sample for DNA analysis and another/different sample for RNA analysis). In addition, the disclosed methods eliminate the requirement of extracting nucleic acid molecules from the samples, prior to analysis. Instead, the sample is simply lysed. The disclosed methods allow for the use of a very small input size compared to standard methods. For example, when RNA and DNA are extracted from an FFPE sample, for example, to perform DNA and RNA sequencing, this normally requires 10-12 tissue sections from the FFPE sample. In contrast, the disclosed methods can use less than 1 FFPE section for analysis of both RNA and DNA. Similarly, the disclosed methods can use only a few thousand cells for analysis of both RNA and DNA (such as lysing only 1000 to 10,000 cells for the analysis, such as 1000 to 5000 cells or 1000 to 2000 cells). Because the methods require less processing of the target nucleic acid molecules, bias, or loss of material (especially loss of small fragments) introduced by such processing can be reduced or eliminated. For example, in some current methods, when the target is both DNA and RNA (such as mRNA and/or miRNA), methods typically employ steps to isolate or extract the nucleic acids from the sample. For example, in prior methods, RNA is typically isolated from a sample, subjected to reverse transcription, amplification, ligation of the RNA, or combinations thereof. Prior methods may also require a depletion or a separation step to remove undesired nucleic acid molecules or undesired library molecules. In some embodiments of the disclosed methods, such steps are not required. As a result, the methods permit one to analyze a range of sample types not otherwise amenable to detection by sequencing. In addition, this results in less loss of the targets from the sample, providing a more accurate result.

The methods can be used to detect DNA and RNA (e.g., sequence, determine the amount of) in the same sample (such as the same individual FFPE tissue section/slice). For example, the methods can be used to detect a mutation, such as one or more nucleotide/ribonucleotide insertions, substitutions, deletions, or combinations thereof, for example gene fusions, insertions, or deletions; tandem repeats, single nucleotide polymorphisms (SNPs); single nucleotide (or ribonucleotide) variants (SNVs); microsatellite repeats; and DNA methylation status. In one example, the methods are used to detect a point mutation in a target nucleic acid molecule. Such a mutation can be a known mutation or a mutation that is newly discovered using the disclosed methods. For example, the methods can be used to detect one or more point mutations (such as at least 2, at least 3, at least 4, at least 5, at least 6, at least 7, at least 8, at least 9, at least 10 or more point mutations, such as 1, 2, 3, 4, 5, 6, 7, 8, 9, or 10 different point mutations) in a single target nucleic acid molecule or in multiple target nucleic acid molecules. The methods can be used to detect an insertion and/or a deletion, such as both an insertation and a deletion (indel, such as one that is less than about 10 kb, less than about 1 kb, less than 100 bases, or less than 50 bases) in a single target nucleic acid molecule or in multiple target nucleic acid molecules. In some examples, each different point mutation is considered a different target nucleic acid molecule. In some examples, the methods can be used to detect one or more point mutations in two or more different target nucleic acid molecules. The method amplifies DNA to generate FAR amplicons to detect target DNA, and uses a nucleic acid probe, referred to herein as a nuclease protection probe comprising a flanking sequence (NPPF), which binds to the target RNA, thereby serving as a surrogate for the target RNA. The method amplifies the ssNPPF to generate NPPF amplicons to detect target RNA. Amplification of the FAR and ssNPPF occurs at the same time, in the same reaction vessel, eliminating the requirement of two separate samples for DNA and RNA analysis. The methods can be multiplexed and, in some examples, roughly conserve the stoichiometry of the sequenced target DNA and RNA molecules.

The primers used to amplify target DNA in the first amplification reaction permit addition of flanking sequences to the resulting FARs, wherein the flanking sequences can be the same as those on the NPPF. The NPPF includes flanking sequences. During the second amplification reaction, sequencing adaptors and/or experiment tags can be added to the FARs and ssNPPFs using the same amplification primers due to the presence of the same flanking sequences. The presence of the experiment tags on the resulting sequencing library (composed of FAR amplicons and NPPF amplicons) permit the identification of the target without necessitating the sequencing of the entire target itself or to permit samples from different patients or different experiments or otherwise to be combined into a single sequencing run. Experiment tags may be included at either the 3′- or the 5′-end or at both ends, for example, to increase multiplexing. Sequencing adaptors permit attachment of a sequence needed for a particular sequencing platform and formation of clusters for some sequencing platforms. The sequencing library composed of FAR amplicons and NPPF amplicons also simplifies the complexity of the sequencer input that is analyzed (e.g., sequenced), as the sequencing library contains a known portion of the target DNA(s) and RNA(s) of interest rather than whole targets, many fragments of whole targets, or unknown targets. The sequencing of FAR amplicons and NPPF amplicons simplifies data analysis compared to that required for other sequencing methods, reducing the algorithm to simply count the amplicons and NPPF amplicons sequenced, rather than having to match sequences to the genome and deconvolute the multiple sequences per gene that are obtained from standard methods of sequencing.

In one example, the disclosure provides methods for sequencing at least one target DNA molecule (by sequencing a FAR amplicon) and at least one RNA molecule (by sequencing an NPPF amplicon) in a sample (such as at least 3, at least 4, at least 5, at least 10, at least 20, at least 30, at least 40, at least 50, at least 100, at least 500, at least 1000, at least 2000, or at least 3000 different target nucleic acid molecules In one example, about 2-100, 2-50, 5-50, 5-100, 50-100, 50-500, 100-1000, 100-2000, 500-3000, 2-40,0000, 2-30,000, 2-20,000, 2-10,000, 100-40,0000, or 30,000-40,000 different target DNA and RNA molecules are analyzed. The sample (e.g., single slice of an FFPE tissue) is lysed and separated or divided into at least two portions (e.g., having the same or a different volume or amount of nucleic acids, such as a volume ratio of the DNA:RNA reaction of at at least about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:30, 1:35, 1:40, 1:45, or 1:50 or 1:1-1:5, 1:1-1:10, 1:10-1:15, 1:15-1:20, 1:10-1:25, 1:10-1:50, or about 1:14; in some examples the DNA reaction has fewer nucleic acid molecules than the RNA reaction or may need more or fewer reads per amplicon of sequencing depth). In some examples, the sample is a fixed sample (such as a paraffin-embedded formalin-fixed (FFPE) sample, hematoxylin and eosin stained tissues, or glutaraldehyde fixed tissues). In some examples, the sample is isolated genomic DNA and isolated RNA obtained from the same sample (e.g., from an individual slice of FFPE tissue section). In some examples, the sample is a single FFPE tissue section (e.g., individual slice), or part of a single (e.g., individual slice) FFPE tissue section. In some examples, the sample contains fewer than 10,000 cells, fewer than 5000 cells, or fewer than 1000 cells, such as 1000-10,000, 1000-5000, 1000-3000, 1000-2000, or 100-1000 cells. For example, the target nucleic acid molecules (e.g., DNA, RNA, or both) can be fixed, cross-linked, or insoluble.

In some examples, the sample (or a portion thereof), such as a sample including nucleic acids (such as DNA and RNA), is heated to denature nucleic acid molecules in the sample, for example to permit subsequent hybridization between target DNA molecules in the sample and at least one target DNA amplification primer (such as a forward and a reverse target DNA amplification primer), and between the NPPF and target RNA molecules in the sample, and hybridization between the NPPF and its corresponding CFS(s).

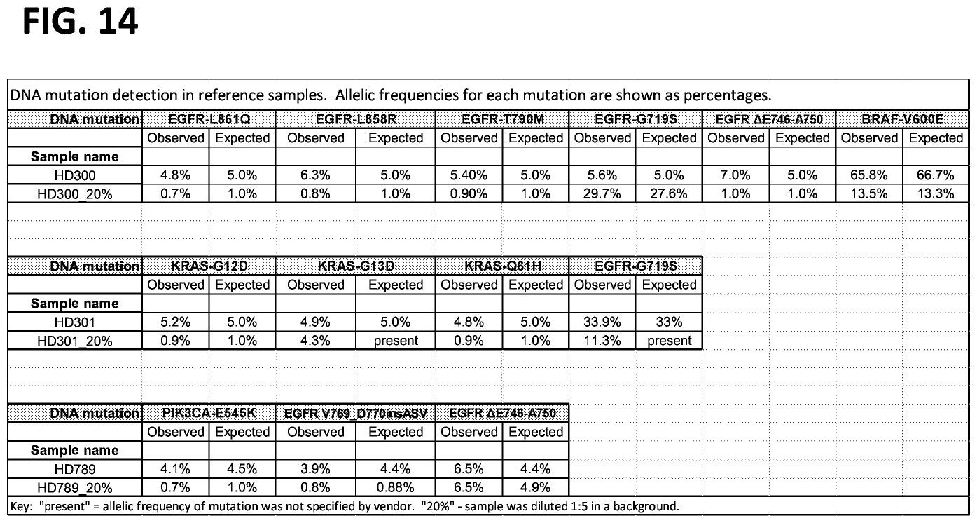

In some examples, the disclosed methods include sequencing at least one target RNA molecule (via an NPPF surrogate) and at least one target DNA molecule in a plurality of samples simultaneously or contemporaneously. Simultaneous sequencing refers to sequencing that occurs at the same time or substantially the same time and/or occurring in the same sequencing library or the same sequencing reaction or performed on the same sequencing flowcell or semiconductor chip (for example, contemporaneous). In some examples, the events occur within 1 microsecond to 120 seconds of one another (for example within 0.5 to 120 seconds, 1 to 60 seconds, or 1 to 30 seconds, or 1 to 10 seconds). In some examples, the disclosed methods sequence two or more target DNA molecules in a sample (e.g., single slice of an FFPE tissue) (for example simultaneously or contemporaneously), for example using (1) at least two different sets of amplification primers in the first amplification step of the target DNA, each set specific for a different target DNA molecule, (2) by using one set of amplification primers specific for a plurality of different target DNA molecules. In one example, at least one portion of the lysed sample is contacted with a plurality of amplification primer sets (such as at least 2, 3, 4, 5, 10, 15, 20, 25, 50, 75, 100, 200, 300, 500, 1000, 2000, 3000, 4000, 5000, or more amplification primer sets), wherein each amplification primer set specifically binds to a particular target DNA molecule. For example, if there are 10 target DNA molecules, at least one portion of the lysed sample can be contacted with 10 different amplification primer sets, each specific for one of the 10 DNA targets. However, in some examples, at least one portion of the lysed sample is contacted with at least one amplification primer set (such as at least 2, 3, 4, 5, 10, 15, 20, 25, 50, 75, 100, 200, 300, 500, 1000, 2000, 3000, 4000, 5000, 10,000, 15,000, 20,000, 25,000, 30,000 or more amplification primer sets), wherein each amplification primer set specifically binds to at least two (such as at least 2, 3, 4, 5, 6, 7, 8, 9, 10, or more) different target DNA molecules (such as a wild type gene and one or more mutations of the wild type gene, such as EGFR, BRAF, PIK3CA, or KRAS). In some examples, at least one portion of the lysed sample is contacted with one or more amplification primer sets that each specifically bind to a particular target DNA molecule and is contacted with one or more amplification primer sets that each specifically bind to at least two different target DNA molecules (such as a wild type gene and one or more mutations of the wild type gene, such as wt EGFR, EGFR with a L861Q mutation, EGFR with a G719S mutation, EGFR with a T790M mutation, and EGFR with an L858R mutation; e.g., see FIG. 14). In some examples, at least 10 different amplification primer sets are incubated with one portion of the lysed sample. However, it is appreciated that in some examples, more than one amplification primer set (such as 2, 3, 4, 5, 10, 20, or more amplification primer sets) specific for a single target DNA molecule can be used, such as a population of amplification primers that are specific for different regions of the same target DNA, or a population of amplification primers that can bind to the target DNA and variations thereof (such as those having mutations or polymorphisms) (for example SEQ ID NOS: 36-43 to detect different EGFR mutations). For example, a particular DNA target known to have multiple polymorphisms of interest across its sequence may have more amplification primers that hybridize to it relative to a DNA target known to have one polymorphism of interest (specific examples provided in Tables 1 and 7). Thus, a population of amplification primer sets can include at least two different amplification primer set populations (such as 2, 3, 4, 5, 10, 20, or 50 different amplification primer sets), wherein each amplification primer population (or sequence) specifically binds to a different target DNA molecule.

In some examples, the disclosed methods sequence two or more target RNA molecules in a sample (e.g., same or individual sample) (for example simultaneously or contemporaneously), for example using (1) at least two different NPPFs, each NPPF specific for a different target RNA molecule, (2) by using one NPPF specific for a plurality of different target RNA molecules. In one example, at least one portion of the lysed sample is contacted with a plurality of NPPFs (such as at least 2, 3, 4, 5, 10, 15, 20, 25, 50, 75, 100, 200, 300, 500, 1000, 2000, 3000, 4000, 5000, 6000, 7000, 8000, 9000, 10,000, 15,000, 20,000, 25,000, 30,000, 35,000, 40,000, 45,000, 50,000 or more NPPFs), wherein each NPPF specifically binds to a particular target RNA molecule. For example, if there are 10 target RNA molecules, at least one portion of the lysed sample can be contacted with 10 different NPPFs each specific for one of the 10 RNA targets. However, in some examples, at least one portion of the lysed sample is contacted with at least one NPPF (such as at least 2, 3, 4, 5, 10, 15, 20, 25, 50, 75, 100, 200, 300, 500, 1000, 2000, 3000, 4000, 5000, 6000, 7000, 8000, 9000, 10,000, 15,000, 20,000, 25,000, 30,000, 35,000, 40,000, 45,000, 50,000 or more NPPFs), wherein each NPPF specifically binds to at least two (such as at least 2, 3, 4, 5, 6, 7, 8, 9, 10, or more) different target RNA molecules (such as separate RNA molecules transcribed from different loci, or more than one alternative transcript or splice isoform transcribed from the same locus). In some examples, the at least one portion of the lysed sample is contacted with one or more NPPFs that each specifically bind to a particular target RNA molecule and is contacted with one or more NPPFs that each specifically bind to at least two different target RNA molecules (such as a wild type RNA and one or more mutations of the wild type RNA). In one example, at least one NPPF is specific for a miRNA target nucleic acid molecule and at least one NPPF is specific for an mRNA target nucleic acid molecule. In some examples, at least 10 different NPPFs are incubated with the sample. However, it is appreciated that in some examples, more than one NPPF (such as 2, 3, 4, 5, 10, 20, or more NPPFs) specific for a single target RNA molecule can be used, such as a population of NPPFs that are specific for different regions of the same target RNA or a population of NPPFs that can bind to the target RNA and variations thereof (such as those with alternative splicing of exons, alternative transcription start sites, tissue-specific isoforms, or structural changes such as insertions, deletions, or fusion transcripts). For example, a low expressed RNA target may have more NPPFs that hybridize to it relative to a RNA target expressed at a higher level, such as four NPPFs hybridizing to a low expressed RNA target and a single NPPF hybridizing to a high expressed RNA target. Thus, a population of NPPFs can include at least two different NPPF populations (such as 2, 3, 4, 5, 10, 20, or 50 different NPPF sequences), wherein each NPPF population (or sequence) specifically binds to a different target RNA molecule.

The methods also include contacting at least one portion of a lysed sample (such as a first portion of a lysed sample) with at least one target DNA amplification primer (such as a set composed of two target DNA amplification primers, such as a forward and reverse primer set) under conditions sufficient for the primer(s) to specifically bind to or hybridize to the target DNA molecule in the lysed sample. In some examples, the target DNA amplification primers include a sequence that allows addition of 5′- and 3′-flanking sequences to the resulting amplicons, wherein the added 5′- and 3′-flanking sequences are identical to the 5′- and 3′-flanking sequences of the NPPF. The methods include contacting at least one portion of a (e.g., single or individual) lysed sample (such as a second portion of a lysed sample) with at least one nuclease protection probe comprising a flanking sequence (NPPF) under conditions sufficient for the NPPF to specifically bind to or hybridize to the target RNA molecule in the lysed sample. Hybridization is the process that occurs wherein there is a sufficient degree of complementarity between two nucleic acid molecules such that stable and specific binding (e.g., base pairing) occurs between the first (e.g., an NPPF or primer) and the second nucleic acid molecule (e.g., a RNA target and CFSs, or DNA target).

The NPPF molecule includes a 5′-end and a 3′-end, as well as a sequence in between that is complementary to all or a part of the target RNA molecule. The 5′-end of a nucleic acid sequence is where the 5′ position of the terminal residue is not bound by a nucleotide. The 3′-end of a nucleic acid molecule is the end that does not have a nucleotide bound to it 3′ of the terminal residue. This permits specific binding or hybridization between the NPPF and the target RNA molecule. For example, the region of the NPPF that is complementary to a region of the target RNA molecule binds to or hybridizes to that region of the target RNA molecule with high specificity. In some examples, the region of the NPPF that is complementary to a region of the target RNA molecule is about 40-150 nt, such as 40-100 nt, 45-60 nt, such as 50 nt (e.g., if the target is mRNA), or about 15-27 nt (e.g., if the target is miRNA). The NPPF molecule further includes one or more flanking sequences, which are at the 5′-end and/or 3′-end of the NPPF. Thus, the one or more flanking sequences are located 5′, 3′, or both, to the sequence complementary to the target nucleic acid molecule. Each flanking sequence includes several contiguous nucleotides, generating a sequence that is not found in a nucleic acid molecule otherwise present in the sample (such as a sequence of at least about 8, 10, 12, 14, 16, 18, 20, 25, 30, or 35 contiguous nucleotides, or about 8-30, 8-25, 8-20, or 10-15 contiguous nucleotides, or at least about 25 contiguous nucleotides). If the NPPF includes a flanking sequence at both the 5′-end and the 3′-end, in some examples the sequence of each NPPF is different and not complementary to each other.

The flanking sequence(s) are complementary to complementary flanking sequences (CFSs) and provide a universal hybridization/amplification sequence, which is complementary to at least a portion of an amplification primer. In some examples, the flanking sequence(s) are identical to the flanking sequence(s) of the FARs. In some examples, the flanking sequence(s) can include (or permit addition of) an experimental tag, sequencing adaptor, or combinations thereof. The methods further include contacting at least one portion of the sample (such as a second portion of the sample) with at least one nucleic acid molecule having complementarity to the flanking sequence (CFS) under conditions sufficient for the CFS to specifically bind or hybridize to the flanking sequence of the NPPF. For example, if the NPPF has a 5′-flanking sequence, at least one portion of the sample is contacted with a nucleic acid molecule having sequence complementarity to the 5′-flanking sequence (5CFS) under conditions sufficient for the 5′-flanking sequence to specifically bind to the 5CFS. Similarly, if the NPPF has a 3′-flanking sequence, at least one portion of the sample is contacted with a nucleic acid molecule having sequence complementarity to the 3′-flanking sequence (3CFS) under conditions sufficient for the 3′-flanking sequence to specifically bind to the 3CFS. One skilled in the art will appreciate that instead of using a single CFS to protect a flanking sequence, multiple CFSs can be used to protect a flanking sequence (e.g., multiple 5CFSs can be used to protect a 5′-flanking sequence). The 5CFS and the 3CFS can be DNA or RNA. In some examples, the 5CFS and/or the 3CFS is an RNA-DNA hybrid oligo, for example wherein the 5′ base or bases of the 5CFS and/or the 3′ base or bases of the 3CFS are RNA, and the remainder of the 5CFS and 3CFS are DNA. In some examples, one or more CFSs contain modifications to a base, or a modification to the 3′ or 5′ end of the CFS, such as a phosphorothioate linkage, a nucleotide that will result in a locked nucleic acid (LNA) (e.g., a ribose s modified with an extra bridge connecting the 2′ oxygen and 4′ carbon), or a chain-terminator (e.g., ddCTP or inverted-T base).

This results in the generation of NPPF molecules that have bound thereto a target RNA molecule (or portion thereof), as well as the CFS(s), thereby generating a double-stranded molecule that includes bases of the NPPF engaged in hybridization to complementary ribobases or bases on the target RNA and CFS. The CFS(s) hybridizes to and, thus, protects its corresponding flanking sequence from digestion with the nuclease in subsequent steps. In some examples, each CFS is the exact length of its corresponding flanking sequence. In some examples, the CFS is completely complementary to its corresponding flanking sequence. However, one skilled in the art will appreciate that the 3′-end of a 5CFS that protects a 5′-end flanking sequence or the 5′-end of a 3CFS that protects the 3′-end flanking sequence can have a difference, such as a nucleotide mismatch, a modification discussed above, or combinations thereof, at each of these positions.

After allowing a target RNA molecule and the CFS(s) to bind to the NPPFs, the method further includes contacting the at least one portion of the sample with a nuclease specific for single-stranded (ss) nucleic acid molecules or ss regions of a nucleic acid molecule, such as S1 nuclease, under conditions sufficient to remove nucleic acid bases (or ribobases) that are not hybridized to complementary bases. Thus for example, NPPFs that have not bound to target RNA molecules or CFSs, as well as unbound single-stranded target RNA molecules, other ss nucleic acid molecules in the sample, and unbound CFSs, are degraded. This generates a digested sample that includes intact NPPFs present as double stranded adducts hybridized to 5CFSs, 3CFSs, or both, and at least a portion of the target RNA. In some examples, the NPPF is composed of DNA and the nuclease includes an exonuclease, an endonuclease, or a combination thereof.

In some examples, the double-stranded (ds) NPPF:target RNA:CFS(s) molecule is separated into its component ss nucleic acid molecules (for example by creating an environment that encourages denaturation, such as heating (e.g., about 95° C. to 100° C. in a buffer or dH₂O), increasing the pH of the sample (e.g. treatment with NaOH), or treatment with 50% formamide/0.02% Tween® detergent), or a combination of such treatments, thereby generating a mixture of ssNPPFs, ss CFSs, and ss target RNA. Such methods allow the liberated NPPF to be further analyzed (such as amplified, sequenced, or both). In some examples, separation of the ds NPPF:target RNA:CFS molecule into its corresponding ss nucleic acid molecules includes treatment with a RNase. Thus, the RNA target is degraded, cleaved, digested, or separated from the NPPF, or combinations thereof, thereby allowing the liberated ssNPPF to be further analyzed (such as amplified, sequenced, or both), thus allowing the ssNPPF to serve as a surrogate of the target RNA. As the ssNPPF is composed of DNA, it can be co-amplified with the DNA amplicons generated in the other portion of the lysed sample. One skilled in the art will appreciate that amplification of the ds NPPF:target RNA:CFS (i.e., the second amplification step) will start with a denaturation step, which may also serve as the method for generating ssNPPFs prior to or during amplification and sequencing.

Thus, the amplicons generated in a first portion of the lysed sample (FARs), and the liberated ssNPPF generated in the second portion of the lysed sample, are combined, and amplified. In some examples, the first portion of the lysed sample and the second portion of the lysed sample are simply combined once the DNA amplicons and liberated ssNPPF are generated, amplification primers added, and the mixture subjected to nucleic acid amplification conditions, such as PCR amplification. In some examples, the volumetric ratio of the second portion of the lysed sample containing liberated ssNPPF to the first portion of the lysed sample containing FARs is 1:1, 1:2, 1:3, 1:4, 1:5, 1:15, 1:10 or 1:20. Such amplification can be used to add an experiment tag and/or sequence adaptor to resulting amplicons, and/or to increase the number of copies of the FARs and the ssNPPFs. At least a portion of the resulting FAR amplicons and NPPF amplicons are sequenced, thereby determining the sequence of the at least one target DNA molecule and the at least one target RNA molecule, respectively in the sample.

The FARs generated in a first portion of the lysed sample, and the liberated ssNPPF generated in the second portion of the lysed sample can be amplified using one or more amplification primers, thereby generating FAR amplicons and NPPF amplicons. One or more of the amplification primers can include a sequence or sequences that act as an experiment tag and/or sequencing adaptor to the FAR amplicons and to the NPPF amplicons. In some examples, one or more of the amplification primers are labeled, such as with a biotin moiety, to permit labeling of the resulting FAR amplicons and NPPF amplicons. In some examples, the FARs and NPPFs have the same flanking sequences, allowing them to be amplified using the same primer or primers.

In one example, at least one of the primers used to amplify the ssNPPF includes a region that is complementary to a flanking sequence of the NPPF. In some examples, two amplification primers are used to amplify the ssNPPF, wherein one amplification primer has a region that has identity to a region of the 5′ flanking sequence and the other amplification primer has a region that is has complementarity to a region of the 3′ flanking sequence, wherein the complementarity is sufficient to allow hybridization of the primers to the ssNPPF. In some examples, the FARs and NPPFs have the same flanking sequences, allowing them to be amplified using the same primers. In some examples, one amplification primer is used (for example to perform linear amplification), wherein the amplification primer has a region that has complementarity to a region of the 3′ flanking sequence.

In some examples, during the co-amplification, both an experiment tag and a sequencing adaptor are added to the FAR and the ssNPPF, for example, at opposite ends of the resulting amplicon(s). For example, the use of such primers can generate an experiment tag and/or sequence adaptor extending from the 5′-end or 3′-end of the amplicons or from both the 3′-end and 5′-end to increase the degree of multiplexing possible. The experiment tag can include a unique nucleic acid sequence that permits identification of a sample, subject, or target nucleic acid sequence. The sequencing adaptor can include a nucleic acid sequence that permits capture of the resulting amplicons onto a sequencing platform. In some examples, primers are removed from the mixture prior to sequencing.

The FAR amplicons and NPPF amplicons are sequenced. Any sequencing method can be used, and the disclosure is not limited to particular sequencing methods. In some examples, the sequencing method used is chain termination sequencing, dye termination sequencing, pyrosequencing, nanopore sequencing, or massively parallel sequencing (also called next-generation sequencing (or NGS)), which is exemplified by ThermoFisher Ion Torrent™ sequencers (e.g. Ion Torrent Personal Genome Machine (PGM™, S5m, or Genexus™ systems), Illumina-branded NGS sequencers (e.g., MiSeg™, NextSeg™) (or as otherwise derived from Solexa™ sequencing) and 454 sequencing from Roche Life Sciences. In some examples, single molecule sequencing is used. In some examples, the method also includes comparing at least one of the obtained sequences of the FAR amplicons or NPPF amplicons to a sequence or mutations database, for example, to determine if a target mutation is present or absent. In some examples, the method includes determining the number of (e.g., counting) each of the FAR amplicons and NPPF amplicons obtained (e.g., wild type, SNPs, newly identified variant, etc.), for example using bowtie, bowtie2, TMAP or other sequence aligners. In some examples, the method includes aligning the sequencing results to an appropriate genome (e.g., if the target nucleic acid molecule(s) are human, then the appropriate genome is the human genome) or portions thereof. In one example, the method includes aligning to only the expected target sequences but enumerating the matches to the expected sequence and any changes within the expected sequence.

### II. Methods of Sequencing

Disclosed herein are methods of sequencing at least one target DNA molecule and at least one target RNA molecule (indirectly via an NPPF surrogate for the RNA) present in a sample, such as a single or individual sample (e.g., a single FFPE slice from a FFPE tissue). In some examples, the at least one target DNA molecule and at least one target RNA molecule (indirectly via an NPPF surrogate) are amplified in the same mixture. In some examples, the same target nucleic acid molecules are detected in at least two different samples or assays (for example, in samples from different patients). Thus, the disclosed methods can be multiplexed (e.g., detecting a plurality of targets in a single sample), high-throughput (e.g., detecting a target in a plurality of samples), or multiplexed and high-throughput (e.g., detecting a plurality of targets in a plurality of samples).

In the disclosed methods, following lysing, the sample (such as a single or individual sample, such as a single FFPE slice from a FFPE tissue) is separated into at least two portions. At least a first portion of the lysed sample is contacted with target DNA-specific primers (such as primers containing flanking sequences), under conditions sufficient for amplification of one or more DNA targets, thus generating FARs. At least a second portion of the lysed sample is contacted with NPPFs and corresponding CFSs under conditions sufficient for hybridization of NPPFs to one or more RNA targets (and CFSs to NPPFs), thus generating an NPPF:target RNA:CFSs complex. The NPPF:target RNA:CFSs complex is then contacted with at least one nuclease specific for ss nucleic acid (such as S1 nuclease) under conditions sufficient for nuclease digestion of ss nucleic acid molecules in the second portion of the lysed sample. The hybridized NPPF:target RNA:CFSs complex is then separated, thus generating ssNPPFs, ssCFSs, and ssRNA. The FARs generated in the first portion of the lysed sample are combined with the ssNPPFs generated in the second portion of the lysed sample, thus generating a mixture of FARs (representing DNA in the sample), ssNPPFs (serving as surrogates of RNA in the sample). The resulting mixture is then incubated with primers under conditions sufficient for amplification of the FARs and ssNPPFs (which can be composed of DNA), thus generating FAR amplicons and NPPF amplicons, which can be sequenced.

In some examples, the ssNPPFs and FARs can be co-amplified in the same reaction mixture, for example, by using primers having a region that is complementary to the flanking sequence(s) of the NPPFs (and can include sequences that allow the incorporation of an experiment tag and/or sequence adaptor to the target) and primers having a region that is complementary to a region of the DNA amplicons (such as flanking sequence(s) added during the first amplification reaction, which is/are, in some examples, identical to the flanking sequences of the NPPFs). In some examples, the disclosed methods provide sequenced nucleic acid molecules that have similar relative quantities of the nucleic acid molecules as in the test sample, such as a variation of no more than 20%, no more than 15%, no more than 10%, no more than 9%, no more than 8%, no more than 7%, no more than 6%, no more than 5%, no more than 4%, no more than 3%, no more than 2%, no more than 1%, no more than 0.5%, or no more than 0.1%, such as 0.001%-5%, 0.01%-5%, 0.1%-5%, or 0.1%-1%.

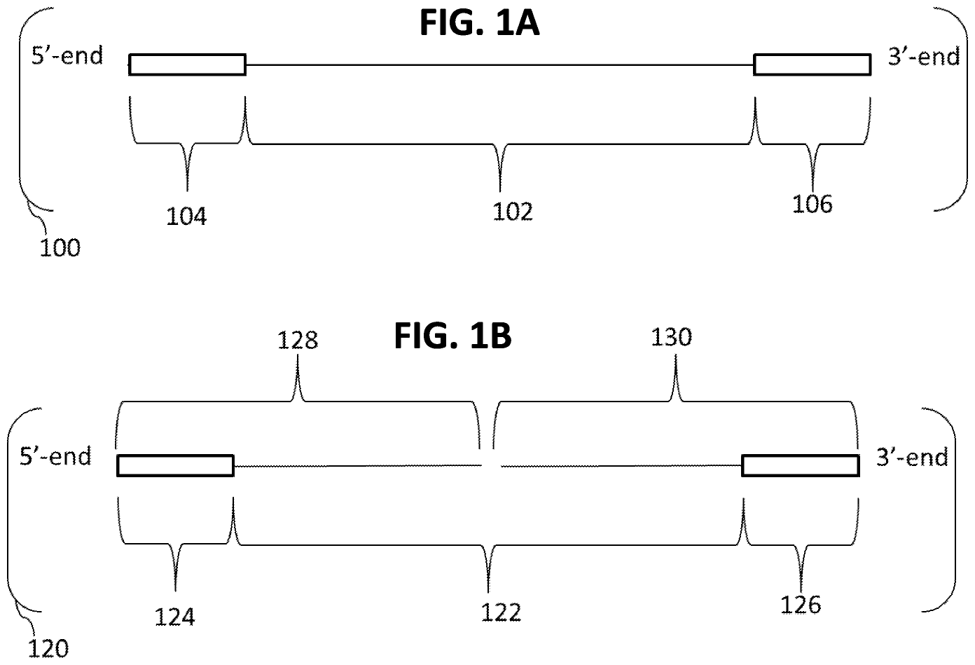

FIGS. 1A and 1B are schematic diagrams showing exemplary NPPFs, which can be used as a “surrogate” for a target RNA. The NPPF functions as a “surrogate” or representative of the target RNA. Thus, if multiple target RNAs are to be detected or sequenced, multiple NPPFs can be used in the disclosed assays. As shown in FIG. 1A, the nuclease protection probe having at least one flanking sequence (NPPF) 100 includes a region 102 that includes a sequence that specifically binds to (e.g., hybridizes to) the target RNA sequence (e.g., at least a portion of the target RNA sequence). The target RNA can be mRNA, miRNA, tRNA, siRNA, rRNA, lncRNA, snRNA, other non-coding RNAs, or combinations thereof. The NPPF includes one or more flanking sequences 104 and 106. FIG. 1A shows an NPPF 100 with both a 5′-flanking sequence 104 and a 3′-flanking sequence 106. However, NPPFs in some examples have only one flanking sequence (e.g., only one of 104 or 106). FIG. 1A shows an exemplary NPPF 100 that is a single nucleic acid molecule. FIG. 1B shows an exemplary NPPF 120 that is composed of two separate nucleic acid molecules 128, 130. For example, if NPPF 100 is a 100-mer, 128, 130 of NPPF 120 could each be a 50-mer. Like the NPPF 100 shown in FIG. 1A, the NPPF 120 includes a region 122 that includes a sequence that specifically binds to (e.g., hybridizes to) the target RNA sequence (e.g., at least a portion of the target RNA sequence), and one or more flanking sequences 124 and 126.

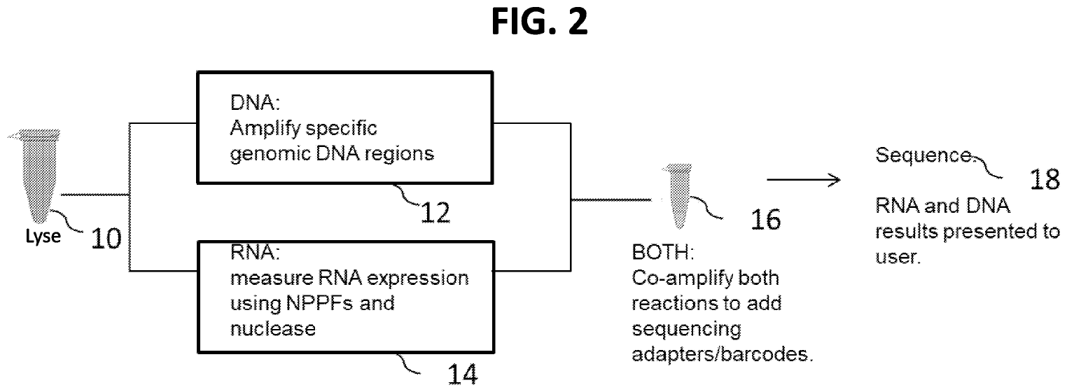

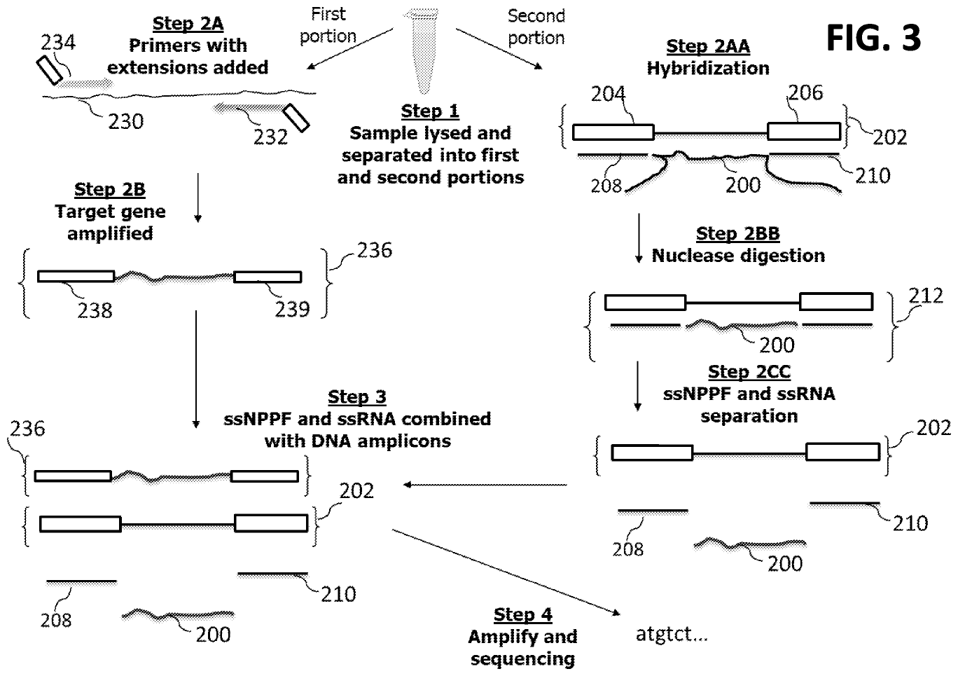

FIG. 2 is a schematic diagram showing an overview of an embodiment of the disclosed methods for nucleic acid amplification of DNA and RNA surrogates in the sample mixture. First, the sample 10 is lysed with a lysis buffer, thereby generating a lysate comprising the target DNA molecule and the target RNA molecule. The resulting lysate is divided or split into at least two fractions or portions, 12, 14. Target DNA in portion 12 is amplified, thereby generating FARs. Target RNA in portion 14 is incubated with NPPFs specific for the target RNA, under conditions that allow the NPPF to specifically bind or hybridize to the target RNA, thereby forming a double stranded (ds) nucleic acid molecule, composed of the NPPF hybridized to the target RNA molecule. The NPPF hybridized to the target RNA molecule complex is incubated with a nuclease specific for single stranded nucleic acid molecules, thereby generating a digested second portion of the lysate comprising NPPF hybridized to the target RNA molecule, and then separating the NPPF from the target RNA. This resulting mixture containing ss NPPF (comprised of DNA) and ss target RNA obtained in portion 14 is mixed with FARs obtained in portion 12, and the mixture 16 subjected to nucleic acid amplification (e.g., PCR), allowing amplification of the FARs and the ss NPPF simultaneously in the same reaction mixture. The resulting amplicons can then be sequenced 18, wherein the NPPF-generated amplicons serve as surrogates for RNA in the sample. A specific example is shown in FIG. 3.

FIG. 3 is a more detailed schematic diagram showing an overview of an embodiment of the disclosed methods for performing amplification with DNA and RNA surrogate in the sample mixture. As shown in Step 1, a sample (such as one known or suspected of containing target RNA 200 and DNA 230) is treated with a sample disruption buffer (e.g., lysed or otherwise treated to make nucleic acids accessible) and then separated into at least two portions. As shown in Step 2A, one portion is used to amplify target DNA, thereby generating FARs 236 (the FARs are double stranded, though only one strand is shown here for simplicity). For example, at least one target DNA 230 is contacted with or incubated with at least one primer (e.g., target DNA primers, such as at least two target DNA primers 234, 232), such as target DNA primers with extensions (for example, to add the same flanking sequences as on the NPPF to the FAR). Target specific primers (e.g., primer pairs) can be used for each target DNA of interest. Thus in some examples, the reaction includes at least two different sets of primers, each set specific for a target DNA (though one will recognize that in some examples a single primer set can amplify multiple DNA targets of interest). As shown in Step 2B of FIG. 3, the target DNA(s) are incubated or contacted with the primers (e.g., target DNA primers) under conditions sufficient for amplification (such as by PCR), thus generating flanked amplicon regions 236. In some examples, amplification of the target DNA in this step utilizes primers that add a 5′- and a 3′-flanking sequence to the FARs, wherein the 5′-end flanking sequence 238 added is the same as the 5′-end flanking sequence of the NPPF 204, and the 3′-end flanking sequence 239 added is the same as the 3′-end flanking sequence of the NPPF 206.

As shown in Step 2AA of FIG. 3, at least a second portion of the sample (i.e., different from the first portion, but still from the same sample) is used to obtain single stranded NPPFs, which serves as a surrogate of the target RNA. For example, the second portion of the lysed sample is contacted with or incubated with a nuclease protection probe having one or more flanking sequences (NPPF) 202 (shown here with both a 5′- and a 3′-flanking sequence, 204 and 206, respectively), which specifically binds to a first target RNA 200. In some examples, the NPPF 202 can bind to a plurality of target RNA molecules, such as different splice isoforms of a particular RNA. The reaction can include additional NPPFs that specifically bind to a second target RNA (or to a plurality of additional target RNA molecules), and so on. In one example, the method uses one or more different NPPFs designed to be specific for each unique target RNA molecule. Thus, the measurement of 100 different RNA targets (e.g., gene expression product(s)) can use at least 100 different NPPFs with at least one NPPF specific per RNA target (such as several different NPPFs/target). In another example, the method uses one or more different NPPFs designed to be specific for a plurality of target RNA molecules, such as different splice isoforms or a wild type RNA and variations thereof. Thus, the measurement of multiple different RNA targets can use a single NPPF. In some examples, combinations of these two types of NPPFs are used in a single reaction. Thus, the method can use at least 2 different NPPFs, at least 3, at least 4, at least 5, at least 10, at least 25, at least 50, at least 75, at least 100, at least 200, at least 500, at least 1000, at least 2000, or at least 2000 different NPPFs (such as 2 to 500, 2 to 100, 2 to 40,000, 2 to 30,000, 2 to 20,000, 2 to 10,000, 2 to 1000, 5 to 10, 2 to 10, 2 to 20, 100 to 500, 100 to 1000, 500 to 5000, 1000 to 3000, 30,000 to 40,000 or 1000 to 30,000 different NPPFs). In addition, one will appreciate that in some examples, a plurality of NPPFs can include more than one (such as 2, 3, 4, 5, 10, 20, 50 or more) NPPFs specific for a single target nucleic acid molecule (which is referred to as a tiled set of NPPFs). The reaction also includes nucleic acid molecules that are complementary to the flanking sequences (CFS) 208, 210. Thus, if the NPPF has a 5′-flanking sequence 204, the reaction will include a sequence complementary to the 5′-flanking sequence (5CFS) 208 and if the NPPF has a 3′-flanking sequence 206, the reaction will include a sequence complementary to the 3′-flanking sequence (3CFS) 210. One skilled in the art will appreciate that the sequence of the CFSs will vary depending on the flanking sequence present. In addition, more than one CFS can be used to ensure a flanking region is protected (e.g., at least two CFSs can use that bind to different regions of a single flanking sequence). The CFS can include natural or unnatural bases and may be RNA or DNA.

In the second portion of the sample (Step 2AA), NPPF(s), and CFS(s) are incubated under conditions sufficient for the NPPFs to specifically bind to (e.g., hybridize to) its respective target RNA molecule, and for CFSs to bind to (e.g., hybridize to) their complementary sequence on the NPPF flanking sequence. In some examples, the CFSs 208, 210 are added in excess of the NPPFs 202, for example at least 2-fold more CFSs than NPPFs (molar excess), such as at least 3-fold, at least 4-fold, at least 5-fold, at least 6-fold, at least 7-fold, at least 8-fold, at least 9-fold, at least 10-fold, at least 20-fold, at least 40-fold, at least 50-fold, or at least 100-fold more CFSs than the NPPFs. In some examples, the NPPFs 202 are added in excess of the total nucleic acid molecules in the sample, for example at least 50-fold more NPPF than total nucleic acid molecules in the sample (molar excess), such as at least 75-fold, at least 100-fold, at least 200-fold, at least 500-fold, or at least 1000-fold more NPPF than the total nucleic acid molecules in the sample. For experimental convenience, a similar concentration of each NPPF can be included to make a cocktail, such that for the most abundant RNA target measured there will be at least 50-fold more NPPF for that RNA target, such as an at least 100-fold excess. The actual excess and total amount of all NPPFs used is limited only by the capacity of the nuclease (e.g., S1 nuclease) to destroy all NPPFs that are not hybridized to target RNA targets. In some examples the reaction at Step 2AA is heated, for example incubated for overnight (such as for 16 hours) at 50° C. This results in the generation of an NPPF hybridized to (1) its target RNA molecule and (2) the 3CFS, 5CFS, or both the 3CFS and the 5CFS.

Following hybridization of the NPPF to its target RNA (and hybridization of the CFSs to their flanking sequence), as shown in Step 2BB in FIG. 3 the sample is contacted with a reagent (such as a nuclease) specific for single-stranded (ss) nucleic acid molecules under conditions sufficient to remove (or hydrolyze or digest) ss nucleic acid molecules, such as unbound nucleic acid molecules (such as unbound NPPFs, unbound CFSs, and unbound target RNA molecules, or portions of such molecules that remain single stranded, such as portions of a target RNA molecule not bound to the NPPF). This results in the generation of a ds NPPF/target RNA/CFSs complex (or duplex) 212. Incubation of the sample with a nuclease specific for ss nucleic acid molecules results in degradation of any ss nucleic acid molecules present, leaving intact double-stranded nucleic acid molecules, including NPPFs that have bound to CFSs and a target RNA molecule. For example, the reaction can be incubated at 50° C. for 1.5 hours with S1 nuclease (though hydrolysis can occur at other temperatures and be carried out for other periods of time, and, in part, the time and temperature required will be a function of the amount of nuclease, and on the amount of nucleic acid required to be hydrolyzed, as well as the Tm of the double-stranded region being protected).

As shown in Step 2CC of FIG. 3, the ds NPPF/RNA target/CFSs complex 212 is exposed to conditions that allow the target RNA sequence 200 and the CFSs (e.g., 5CFS 208, 3CFS 210, or both) to be separated from the NPPF, thereby generating ssRNA 200, ssNPPF 202, and ssCFSs (such as 208 and 210). Although two CFSs are shown, single CFS embodiments are also contemplated by this disclosure. If only one flanking sequence is present on the NPPF, only one CFS will have been bound in the NPPF/target complex. The CFSs can be DNA or RNA (or a mixture of both nucleotide types). In one example, 5CFS 208 and/or 3CFS 210 are DNA. In some examples, the reaction can be heated or the pH altered (e.g., to result in the reaction having a basic pH) under conditions that allow the NPPF 202 to dissociate from the hybridized RNA target 200, resulting in a mixed population of ssNPPFs 202 and ss target nucleic acids (e.g., ssRNA targets) 200. In some examples, Step 2CC of FIG. 3 is performed as the first step of Step 4 of FIG. 3, that is instead of performing a separate denaturation step, the ds NPPF/RNA target/CFSs complex 212 is dissociated into ss nucleic acid molecules as the first step in the second amplification reaction.

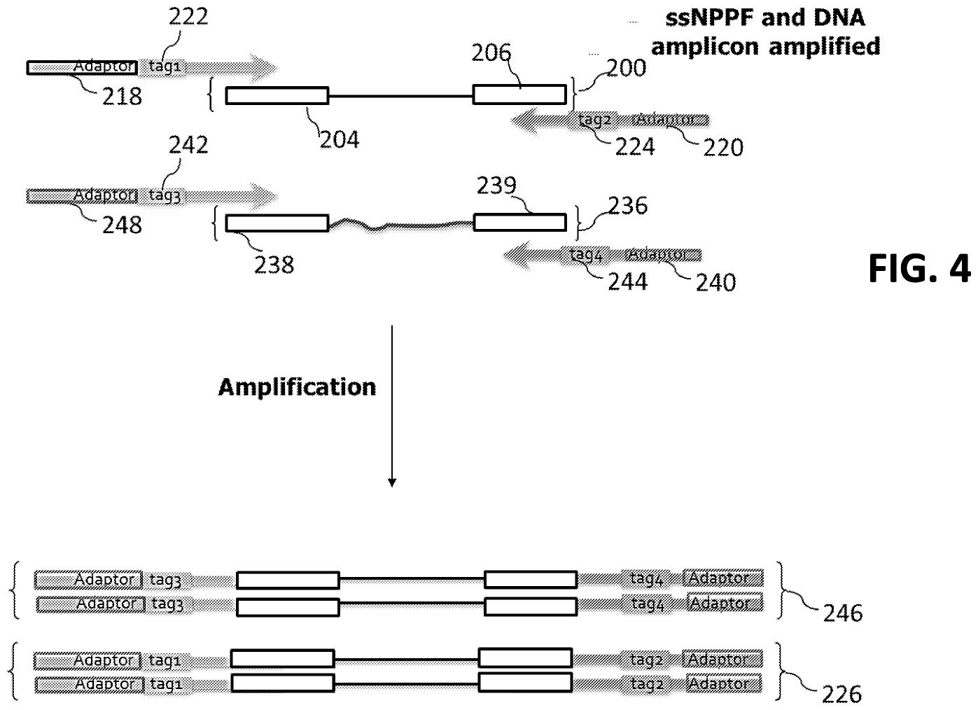

As shown in Step 3 of FIG. 3, the mixture obtained after Step 2CC containing ssNPPFs 202 (or the ds NPPF/RNA target/CFSs complex 212 obtained after step 2BB of FIG. 3), and the mixture obtained after Step 2B containing FARs (which are double stranded) 236 are combined into a single mixture. As shown in Step 4 of FIG. 3, the resulting mixture is subjected to nucleic acid amplification conditions (e.g., using PCR) to generate amplicons, prior to the sequencing. Thus, FARs and the ssNPPF (or RNA ds NPPF/RNA target/CFSs complex 212) surrogates are amplified in the same reaction, generating amplicons, which can then be sequenced. FIG. 4 shows exemplary PCR primers or probes as arrows, which can be used in the amplification reaction shown in Step 4. The PCR primers or probes can include one or more experiment tags 222, 224, 242, 244 (e.g., that allow for the identification of a sample or patient) and/or sequencing adaptors 218, 220, 248, 240 (e.g., that allow the targets to be sequenced by a particular sequencing platform, and, thus, such adaptors are complementary to capture sequences on e.g. a sequencing chip or flowcell). At least a portion of the PCR primers/probes are specific for the flanking sequences 204, 206 (and in some examples also 238, 239). In some examples, the concentration of the primers are in excess of the ssNPPFs 200 and/or the FARs 236, for example, in excess by at least 10,000-fold, at least 50,000-fold, at least 100,000-fold, at least 150,000-fold, at least 200,000-fold, at least 400,000-fold, at least 500,000-fold, at least 600,000-fold, at least 800,000-fold, or at least 1,000,000-fold. In some examples, the concentration of primers 208 in the reaction is at least 200 nM (such as at least 400 nM, at least 500 nM, at least 600 nM, at least 750 nM, or at least 1000 nM).

In Step 4 of FIG. 3, amplicons generated in Step 3 can be sequenced. In some examples, a plurality of FAR amplicons and NPPF amplicons are sequenced in parallel, for example, simultaneously or contemporaneously. Thus, this method can be used to sequence a plurality of target nucleic acid sequences.

A. Exemplary Hybridization Conditions

Disclosed herein are conditions sufficient for (1) amplification primers to specifically hybridize to their complementary nucleic acid molecules (e.g., to DNA target molecules in a lysed sample, to FARs, and to ssNPPFs), and (2) an NPPF or a plurality of NPPFs to specifically hybridize to target RNA molecule(s), such RNAs present in a at least one portion of a lysed sample, as well as specifically hybridize to CFS complementary to the flanking sequence(s). In some examples, a plurality of NPPFs include at least 2, at least 5, at least 10, at least 20, at least 100, at least 500, at least 1000, at least 3000, at least 10,000, at least 15,000, at least 20,000, at least 25,000, at least 30,000, at least 35,000, at least 40,000, at least 45,000, or at least 50,000 (such as 2 to 5000, 2 to 3000, 10 to 1000, 50 to 500, 25 to 300, 50 to 300, 10 to 100, 50 to 100, 500 to 1000, 1000 to 5000, 2000 to 10,000, 100 to 50,000, 100 to 40,000, 100 to 30,000, 100 to 20,000, 100 to 10,000, 10 to 50,000, 10 to 40,000, 10 to 30,000, 10 to 20,000, 10 to 10,000, or 30,000 to 40,000) unique NPPF sequences.

Hybridization is the ability of complementary single-stranded DNA, RNA, or DNA/RNA hybrids, to form a duplex molecule (also referred to as a hybridization complex). For example, the features (such as length, base composition, and degree of complementarity) that will enable a nucleic acid (e.g., an NPPF) to hybridize to another nucleic acid (e.g., target RNA or CFS) under conditions of selected stringency, while minimizing non-specific hybridization to other substances or molecules can be determined based on the present disclosure. “Specifically hybridize” and “specifically complementary” are terms that indicate a sufficient degree of complementarity such that stable and specific binding occurs between a first nucleic acid molecule (e.g., an NPPF or primer) and a second nucleic acid molecule (such as a nucleic acid target, for example, a DNA or RNA target, or a CFS). The first and second nucleic acid molecules need not be 100% complementary to be specifically hybridizable. Specific hybridization is also referred to herein as “specific binding.” Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11).

Characteristics of the NPPFs are discussed in more detail in Section III, below. Typically, a region of an NPPF will have a nucleic acid sequence (e.g., FIG. 1A, 102) that is of sufficient complementarity to its corresponding target RNA molecule(s) to enable it to hybridize under selected stringent hybridization conditions, as well as a region (e.g., FIG. 1A, 104, 106) that is of sufficient complementarity to its corresponding CFS(s) to enable it to hybridize under selected stringent hybridization conditions. In some examples, an NPPF shares at least 90%, at least 92%, at least 95%, at least 98%, at least 99% or 100% complementarity to its target RNA sequence(s). Exemplary hybridization conditions include hybridization at about 37° C. or higher (such as about 37° C., 42° C., 50° C., 55° C., 60° C., 65° C., 70° C., 75° C., or higher, such as 45-55° C. or 48-52° C.). Among the hybridization reaction parameters which can be varied are salt concentration, buffer, pH, temperature, time of incubation, amount and type of denaturant such as formamide. For example, nucleic acid (e.g., a plurality of NPPFs) can be added to at least one portion of a sample at a concentration ranging from about 10 pM to about 10 nM (such as about 30 pM to 5 nM, about 100 pM to about 1 nM, such as 1 nM NPPFs), in a buffer (such as one containing NaCl, KCl, H₂PO₄, EDTA, 0.05% Triton X-100, or combinations thereof) such as a lysis buffer.

In some examples, the NPPFs are added in excess of the corresponding target RNA molecules in at least one portion of the sample, such as an at least 10-fold, at least 50-fold, at least 75-fold, at least 100-fold, at least 250-fold, at least 1,000 fold, at least 10,000 fold, at least 100,000 fold, at least 1,000,000 fold, or at least 10,000,000 fold molar excess or more of NPPF to corresponding target RNA molecules in the at least one portion of the sample. In one example, each NPPF is added to the at least one portion of the sample at a final concentration of at least 10 pM, such as at least 20 pM, at least 30 pM, at least 50 pM, at least 100 pM, at least 150 pM, at least 200 pM, at least 500 pM, at least 1 nM, or at least 10 nM. In one example, each NPPF is added to the at least one portion of the sample at a final concentration of about 125 pM. In another example, each NPPF is added to the at least one portion of the sample at a final concentration of about 167 pM. In a further example, each NPPF is added to the at least one portion of the sample at a final concentration of about 1 nM. In a further example, each NPPF is added to the at least one portion of the sample at least about 100,000,000, at least 300,000,000, or at least about 3,000,000,000 copies per μl. In some examples, the CFSs are added in excess of the NPPFs, such as an at least 2-fold, at least 3-fold, at least 4-fold, at least 5-fold or at least 10-fold molar excess of CFS to NPPF. In one example, each CFS is added to the at least one portion of the sample at a final concentration of about at least 6 times the amount of probe, such as at least 10 times or at least 20 times the amount of probe (such as 6 to 20 times the amount of probe). In one example, each CFS (e.g., 5CFS and 3CFS) is added at least at 1 nM, at least 5 nM, at least 10 nM, at least 50 nM, at least 100 nM, or at least 200 nm, such as 1 to 100, 5 to 100 or 5 to 50 nM. For example if there are six probes, each at 166 pM, each CFSs can be added at 5 to 50 nM.

Prior to hybridization with NPPFs and CFS(s), the nucleic acids in at least one portion of the sample are denatured, rendering them single stranded and available for hybridization (for example at about 85° C. to about 105° C. for about 5-15 minutes, such as 85° C. for 10 minutes). By using different denaturation solutions, this denaturation temperature can be modified, so long as the combination of temperature and buffer composition leads to formation of single stranded target DNA or RNA or both.

In the portion of the lysed sample used to obtain NPPFs for surrogates of RNA present in the sample, the nucleic acids in the at least one portion of the lysed sample and the 5CFS, 3CFS, or both, are hybridized to the plurality of NPPFs for between about 10 minutes and about 72 hours (for example, at least about 1 hour to 48 hours, about 2 to 16 hours, about 6 hours to 24 hours, about 12 hours to 18 hours, about 16 hours, or overnight, such as 2 to 20 hours) at a temperature ranging from about 4° C. to about 70° C. (for example, about 37° C. to about 65° C., about 42° C. to about 60° C., or about 50° C. to about 60° C., such as 50° C.). In one example, hybridization is performed at 50° C. for 2 to 20 hours. Hybridization conditions will vary depending on the particular NPPFs and CFSs used, but are set to ensure hybridization of NPPFs to the target RNA molecules and the CFSs. In some examples, the plurality of NPPFs and CFSs are incubated with the at least one portion of the lysed sample at a temperature of at least about 37° C., at least about 40° C., at least about 45° C., at least about 50° C., at least about 55° C., at least about 60° C., at least about 65° C., or at least about 70° C. In one example, the plurality of NPPFs and CFSs are incubated with the sample at about 37° C., at about 42° C., or at about 50° C.

In some embodiments, the methods do not include nucleic acid purification (for example, nucleic acid purification is not performed before or after lysis of the sample, such as not prior to contacting a portion of the lysed sample with NPPFs and CFSs or with nucleic acid primers for target DNA amplification, and/or nucleic acid purification is not performed following contacting the sample with the NPPFs and CFSs, or with nucleic acid primers for target DNA amplification). In some examples, no pre-processing of the sample is required except for cell lysis. In some examples, cell lysis and contacting the sample with either (1) primers to amplify target DNA or (2) the plurality of NPPFs and CFSs, occur sequentially.

B. Treatment with Nuclease

As shown in Step 2BB of FIG. 3, following hybridization of the NPPFs to target RNA and to CFS(s), at least one portion of the lysed sample is subjected to a nuclease protection procedure. The target RNA molecules and CFSs (one or two CFSs, depending if there are both 5′- and 3′-flanking sequence on the NPPF or just one) that have hybridized to the NPPF are not hydrolyzed by the nuclease and can be subsequently amplified and/or sequenced.

Nucleases are enzymes that cleave a phosphodiester bond. Endonucleases cleave an internal phosphodiester bond in a nucleotide chain (in contrast to exonucleases, which cleave a phosphodiester bond at the end of a nucleotide chain). Thus, endonucleases, exonuclease, and combinations thereof, can be used in the disclosed methods. Endonucleases include restriction endonucleases or other site-specific endonucleases (which cleave DNA at sequence specific sites), DNase I, pancreatic RNAse, Bal 31 nuclease, S1 nuclease, mung bean nuclease, Ribonuclease A, Ribonuclease T1, RNase I, RNase PhyM, RNase U2, RNase CLB, micrococcal nuclease, and apurinic/apyrimidinic endonucleases. Exonucleases include exonuclease III and exonuclease VII. In particular examples, a nuclease is specific for single-stranded nucleic acids, such as S1 nuclease, P1 nuclease, mung bean nuclease, or BAL 31 nuclease. Reaction conditions for these enzymes are known and can be optimized empirically.

Treatment with one or more nucleases can destroy all ss nucleic acid molecules (including RNA and DNA in the lysed sample that is not hybridized to (thus, not protected by) NPPFs, NPPFs that are not hybridized to target RNA, and CFSs not hybridized to an NPPF), but will not destroy ds nucleic acid molecules such as NPPFs that have hybridized to CFSs and a target nucleic acid molecule present in the at least one portion of the lysed sample. For example, unwanted nucleic acids, such as one or more non-target DNA (such as genomic DNA, cDNA) and non-target RNA (e.g., non-target, tRNA, rRNA, mRNA, miRNA), and portions of the target RNA molecule(s) that are not hybridized to complementary NPPF sequences (such as overhangs), which, in the case of mRNA targets, will constitute the majority of the nucleic target sequence, can be substantially destroyed in this step. In some embodiments, this step leaves behind approximately a stoichiometric amount of target RNA/CFS/NPPF duplex. If the target RNA molecule is cross-linked to tissue that occurs from fixation, the NPPFs hybridize to the cross-linked target RNA molecule without the need, in some embodiments, to reverse cross-linking, or otherwise release the target nucleic acid from the tissue to which it is cross-linked.

In some examples, S1 nuclease diluted in a buffer (such as one containing sodium acetate, NaCl, KCl, ZnSO₄, an antimicrobial agent (such as ProClin™ biocide), or combinations thereof) is added to the hybridized NPPF/target RNA/CFS sample mixture and incubated at about 37° C. to about 60° C. (such as about 50° C.) for 10-120 minutes (for example, 10-30 minutes, 30 to 60 minutes, 60-90 minutes, 90 minutes, or 120 minutes) to digest non-hybridized nucleic acid from the at least one portion of the lysed sample and non-hybridized NPPFs, RNAs, and CFSs. In one example, the nuclease digestion is performed by incubating the at least one portion of the lysed sample with the nuclease in a nuclease buffer at 50° C. for 60 to 90 minutes.

Following nuclease digestion, the at least one portion of the lysed sample can optionally be treated to inactivate or remove residual enzymes (e.g., by phenol extraction, precipitation, column filtration, addition of proteinase K, addition of a nuclease inhibitor, chelating divalent cations required by the nuclease for activity, heating, or combinations thereof). In some examples the at least one portion of the lysed sample is treated to adjust the pH to about 7 to about 8, for example, by addition of KOH or NaOH or a buffer (such as one containing Tris-HCl at pH 9 or Tris-HCl at pH 8). Raising the pH can prevent the depurination of DNA and prevents many ss-specific nucleases (e.g., S1) from functioning fully. In some examples, the at least one portion of the lysed sample is heated (for example 80-100° C.) to inactivate the nuclease, for example for 10-30 minutes.

C. Separation of ssNPPFs from the Target Nucleic Acids

As shown in Step 2CC of FIG. 3, following nuclease treatment of the at least one portion of the lysed sample containing the double-stranded NPPF/target RNA/CFSs complexes, the NPPFs are separated (e.g., denatured) from the ss nucleic acid target and the CFS(s). Thus, the double-stranded NPPF/target RNA/CFSs complex can be separated into single-stranded nucleic acid molecules, the ssNPPF and the ss target nucleic acid (e.g., ssRNA) (as well as the ss CFSs).

In some examples, Step 2CC of FIG. 3 is performed as the first step of Step 4 of FIG. 3. For example, instead of performing a separate denaturation/separation step, the ds NPPF/RNA target/CFSs complex 212 is dissociated into ss nucleic acid molecules as the first step in the second amplification reaction (e.g., the first step of Step 4 in FIG. 3).

D. Nucleic Acid Amplification

As shown in FIG. 3, the method includes two nucleic acid amplification steps, using methods such as polymerase chain reaction (PCR) or other forms of enzymatic amplification. The first amplification amplifies target DNA in a portion of the lysed sample (Step 2B). The second amplification amplifies the FARs resulting from the first amplification along with the ss NPPFs obtained after hybridization, nuclease digestion, and denaturation (Step 4).

In some examples, no more than 30 cycles of amplification are performed at each amplification step, such as no more than 25 cycles of amplification, no more than 20 cycles of amplification, no more than 15 cycles of amplification, no more than 10 cycles of amplification, no more than 8 cycles of amplification, or no more than 5 cycles of amplification, such as 2 to 30 cycles, 5 to 30 cycles, 8 to 30 cycles, 8 to 25 cycles, 2 to 25 cycles, 5 to 25 cycles, 5 to 20 cycles, 5 to 15 cycles, or 5 to 10 cycles of amplification for each amplification step.

During the first amplification step (amplification of target DNA), the least number of cycles of amplification needed is used, to reduce the number of errors introduced during the amplification. In some examples, 5 to 20 amplification cycles are performed in the first amplification, such as 5 to 15 cycles, such as 5, 6, 7, 8, 9, 10, 11, 12, 13, 14, 15, 16, 17, 18, 19, or 20 cycles. In some examples, 10 amplification cycles are performed in the first amplification. In some examples, the primers used in the first amplification step have a Tₘ of about 50-62° C. In some examples, the annealing temperature used in the first amplification reaction is at least 50° C., at least 56° C., or at least 58° C., such as about 50° C. to 60° C., such as about 56° C. to 60° C., such as about 52° C. to 58° C., such as 56° C., 57° C. or 58° C. In some examples, the FARs generated from the first amplification step are about 70 to 200 bp, such as 70 to 150, 70 to 125, 90 to 150 bp, such as about 70 bp, about 100 bp, or about 140 bp.

In some examples, during the second amplification step (amplification of FARs and ssNPPFs), 8 to 30 amplification cycles are performed in the second amplification, such as 15 to 25 or 8 to 25 cycles, such as 8, 9, 10, 11, 12, 13, 14, 15, 16, 17, 18, 19, 20, 21, 22, 23, 24, 25, 26, 27, 28, 29 or 30 cycles. In some examples, 19 amplification cycles are performed in the second amplification. In some examples, the primers used in the second amplification step have a Tm of about 50-62° C. In some examples, the annealing temperature used in the second amplification reaction is at least 50° C., at least 56° C., or at least 56° C., such as about 50° C. to 60° C., such as about 52° C. to 58° C., such as 56° C. In some examples, the FAR amplicons generated from the second amplification step are about 150 to 250 bp, such as 150 to 200 bp, such as about 180 bp. In some examples, the NPPF amplicons generated from the second amplification step are about 150 to 250 bp, such as 150 to 200 bp, such as about 155 bp or 180 bp.

In some examples, portion of an amplification primer that anneals to its target is about 15-25 nt (such as 22 nt) with about 50% GC content. In some examples, an amplification primer is about 50 to 100 nt (such as 60 to 100 nt) in length.

Nucleic acid amplification methods that can be used include those that result in an increase in the number of copies of a nucleic acid molecule, such as a target DNA (or amplicon thereof), target RNA surrogate (i.e., indirectly by amplification of ssNPPF), and/or portion thereof. The resulting products are called amplification products or amplicons. Generally, such methods include contacting material to be amplified (e.g., target DNA (or amplicon thereof) or ssNPPF) with one or a pair of oligonucleotide primers, under conditions that allow for hybridization of the primer(s) to the nucleic acid molecule to be amplified. The primers are extended under suitable conditions, dissociated from the template, and then re-annealed, extended, and dissociated to amplify the number of copies of the nucleic acid molecule.

Examples of in vitro amplification methods that can be used include, but are not limited to, PCR, quantitative real-time PCR, isothermal amplification methods, strand displacement amplification; transcription-free isothermal amplification; repair chain reaction amplification; and NASBA™ RNA transcription-free amplification. In one example, the primers specifically hybridize to at least a portion of the NPPF flanking sequence(s). In one example, helicase-dependent amplification is used.

During the second amplification of the FARs and ssNPPFs, an experiment tag, and/or sequencing adaptor can be incorporated as, for instance, part of the primer (see FIG. 4). However, addition of such tags/adaptors is optional. For example, an amplification primer, which includes a first portion that is complementary to all or part of a 5′- or 3′-flanking sequence (e.g., 238, 239, 204, 206 of FIG. 3), can include a second portion that is complementary to a desired experiment tag and/or sequencing adaptor. One skilled in the art will appreciate that different combinations of experiment tags and/or sequencing adaptors can be added to either end of the FAR or ssNPPF.

In one example, DNA in the lysed sample is amplified using a first primer that includes a first portion complementary to all or a portion of the target DNA sequence and a second portion complementary to (or comprising) a desired flanking sequence (e.g., complementary to the 5′-flanking sequence of the NPPF) and with a second primer that includes a first portion complementary to all or a portion of the target DNA sequence and a second portion complementary to (or comprising) a desired flanking sequence (e.g., complementary to the 5′-flanking sequence of the NPPF) (see FIG. 3, Step 2A), such that the flanking sequence 238, 239 becomes incorporated into the resulting amplicon (see FIG. 3, Step 2B). In one example, two different flanking sequences are used.

In one example, the FAR and the ssNPPF are amplified using a first amplification primer that includes a first portion complementary to all or a portion of the 5′ flanking sequence and a second portion complementary to (or comprising) a desired sequencing adaptor, and the second amplification primer includes a first portion complementary to all or a portion of the 3′ flanking sequence and a second portion complementary to (or comprising) a desired experiment tag (e.g., see FIG. 4). In one example, two different sequencing adapters and two different experiment tags are used. In some examples, two different sequencing adapters and one experiment tag are used. In another example, the FAR and the ssNPPF is amplified using a first amplification primer that includes all or a portion of a first portion identical to (or complementary to) the 5′ flanking sequence and a second portion complementary to (or comprising) a desired sequencing adaptor and a desired experiment tag, and the second amplification primer includes a first portion complementary to all or a portion of the 3′ flanking sequence and a second portion complementary to (or comprising) a desired experiment tag.

Amplification can also be used to introduce a detectable label into the generated target nucleic acid amplicons (for example, if additional labeling is desired) or other molecule that permits detection or quenching. For example, the amplification primer can include a detectable label, hapten, or quencher that is incorporated into the target nucleic acid amplicons during amplification. Such a label, hapten, or quencher can be introduced at either end of the target amplicon(s) (or both ends) or anywhere in between.

In some examples, the resulting FAR amplicons and NPPF amplicons are purified before sequencing. For example, the amplification reaction mixture can be purified before sequencing using methods known in the art (e.g., gel purification, biotin/avidin capture and release, capillary electrophoresis, size-exclusion purification, or binding to and release from paramagnetic beads (solid phase reversible immobilization)). In one example, the FAR amplicons and NPPF amplicons are biotinylated (or include another hapten) and captured onto an avidin or anti-hapten coated bead or surface, washed, and then released for sequencing. Likewise, the FAR amplicons and NPPF amplicons can be captured onto a complimentary oligonucleotide (such as one bound to a surface), washed and then released for sequencing. The capture of amplicons need not be particularly specific, as the disclosed methods eliminate most of the genome or transcriptome, leaving the desired amplicons. Other methods can be used to purify the FAR amplicons and NPPF amplicons, if desired.

The FAR amplicons and NPPF amplicons can also be purified after the last step of amplification, while still double stranded, by a method which uses a nuclease that hydrolyzes single stranded oligonucleotides (such as Exonuclease I), which nuclease can in turn be inactivated before continuing to the next step such as sequencing.

1. Primers

The amplification primers that specifically bind or hybridize to the flanking sequence(s) (e.g., 5′ and/or 3′ flanking sequence(s) of the NPPF and FARs), as well as those specific for the target DNA, can be used to initiate amplification, such as PCR amplification. Thus, primers having sequence complementarity to the flanking sequence can anneal to an NPPF by nucleic acid hybridization to form a hybrid between the primer and the flanking sequence of the surrogate NPPF, and then the primer extended along the complement strand by a polymerase enzyme. Similarly, primers having sequence complementarity to the target DNA can anneal to the target DNA by nucleic acid hybridization to form a hybrid between the primer and the target DNA, and then the primer extended along the complement strand by a polymerase enzyme.

In addition, the amplification primers can be used to introduce nucleic acid markers (such as one or more experiment tags and/or sequencing adaptors) and/or detectable labels to the resulting target nucleic acid amplicons.

Primers are short nucleic acid molecules, such as a DNA oligonucleotides that are at least 12 nucleotides in length (such as about 15, 20, 25, 30, 50, 60, 61, 62, 63, 64, 65, 66, 67, 68, 69, 70, 75 or 80 nucleotides or more in length, such as 15 to 25 nt, 50 to 80 nt, 60-70 nt or 60-66 nt).

For the first amplification, primers in some examples include a region of about 15-25 nt that has complementarity to the target DNA, and another 25 nt extension on one end (e.g., complementary to a 5′- or 3′-flanking sequence of an NPPF).

For the second amplification, primers in some examples include a region of about 15-25 nt that has complementarity to the 5′- or 3′-flanking sequence of an NPPF or FAR, and a region having a nucleic acid sequence that allows for the addition of a sequence adaptor, experiment tag, or both to the resulting amplicons. It can also include a region having a nucleic acid sequence that results in addition of a detectable label to the resulting amplicon. An experiment tag and/or sequencing adaptor can be introduced at the 5′- and/or 3′-end of the amplicon. In some examples, two or more experiment tags and/or sequencing adaptors are added to a single end or both ends of the amplicon, for example using a single primer having a nucleic acid sequence that results in addition of two or more experiment tags and/or sequencing adaptors. Experiment tags can be used, for example, to differentiate one sample or sequence from another. Sequence adaptors permit capture of the resulting amplicon by a particular sequencing platform.

2. Addition of Experiment Tags

Experiment tags are short sequences or modified bases that serve as an identifier for one or several reactions to be independently discerned by, for example: patient, sample, cell type, time course timepoint, or treatment. Experiment tags can be part of the flanking sequence of the NPPF and the FAR. In another example, the experiment tag is added during amplification (e.g., amplification of the FAR and ssNPPF), resulting in an amplicon (e.g., FAR amplicon and NPPF amplicon) containing an experiment tag. The presence of universal sequences in the flanking sequence(s) permit the use of universal primers, which can introduce other sequences onto the NPPF amplicons, for example during amplification. Experimental tags can also be used for amplification, such as nested amplification, or two stage amplification. Exemplary experiment tags are provided in Tables 3-5.

Experiment tags, such as one that differentiates one sample from another, can be used to identify the particular target sequence. Thus, experiment tags can be used to distinguish experiments or patients from one another. In one example, the experiment tag is the first three, five, ten, twenty, or thirty nucleotides of the 5′- and/or 3′-end of a resulting amplicon. In some examples, the experiment tags are placed in proximity to the sequencing primer site. For Illumina® sequencing, experiment tags are immediately next to the Read 1 and Read 2 primer sites. For some sequencing platforms, experiment tags are generally the first few bases read. In particular examples, the experiment tag is at least 3 nucleotides in length, such as at least 5, at least 10, at least 15, at least 20, at least 25, at least 30, at least 40, or at least 50 nucleotides in length, such as 3-50, 3-20, 12-50, 6-8, 8-10, 6-12, or 12-30 nucleotides, for example, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotides in length.

In one example, an experiment tag is used to differentiate one sample from another. For example, such a sequence can function as a barcode, to allow one to correlate a particular sequence detected with a particular sample, patient, or experiment (such as a particular reaction well, day, or set of reaction conditions). This permits a particular target nucleic acid amplicon that is sequenced to be associated with a particular patient or sample or experiment for instance. The use of such tags provides a way to lower cost per sample and increase sample throughput, as multiple target nucleic acid amplicons can be tagged and then combined (for example, from different experiments or patients), for example, in a single sequencing run or detection array. This allows for the ability to combine different experimental or patient samples into a single run within the same instrument channel or sequencing consumable (such as a flowcell or a semiconductor chip). For example, such tags permitting 100s or 1,000s of different experiments to be sequenced in a single run within a single flowcell or chip. In addition, if the method includes the step of gel purifying the completed amplification reaction (or other method of purification or clean up that does not require actual separation) only one gel (or clean up or purification reaction or process) needs to be run per detection or sequencing run. Similarly, if sequencing requires a quantitation step, then either individual samples or only the pool of samples may be quantitated prior to sequencing. The sequenced target nucleic acid amplicons can then be sorted, for example, by the experiment tags.

In one example, the experiment tag is used to identify the particular target sequence. In this case, using an experimental tag to correspond to a particular target sequence can shorten the time or amount of sequencing needed, as sequencing the end of the target nucleic acid amplicon instead of the entire target nucleic acid amplicon can be sufficient. For example, if such an experiment tag is present on the 3′-end of the target nucleic acid amplicon, the entire target nucleic acid amplicon sequence itself does not have to be sequenced to identify the target sequence. Instead, only the 3′-end of the target nucleic acid amplicon containing the experiment tag needs to be sequenced. This can significantly reduce sequencing time and resources, as less material needs to be sequenced.

3. Addition of Sequencing Adaptors

Sequencing adaptors can, but need not, be part of the flanking sequence(s) of the NPPFs and FARs when generated. In another example, a sequencing adaptor is added during amplification of a nucleic acid (e.g., amplification of the FAR or ssNPPFs), resulting in amplicons containing a sequencing adaptor. The presence of a universal sequence in the flanking sequence(s) permit the use of universal amplification primers, which can introduce other sequences onto the NPPF surrogate and FAR, for example during amplification.

A sequencing adaptor can be used add a sequence to a nucleic acid (e.g., FAR and surrogate NPPFs) needed for a particular sequencing platform. For example, some sequencing platforms (such as the 454-branded (Roche), Ion Torrent-branded and Illumina-branded) require the nucleic acid molecule to be sequenced to include a particular sequence at its 5′- and/or 3′-end, for example, to capture the molecule to be sequenced. For example, the appropriate sequencing adaptor is recognized by a complementary sequence on the sequencing chip or beads, and the amplicon captured by the presence of the sequencing adaptor.

In one example, a poly-A (or poly-T), such as a poly-A or poly-T at least 10 nucleotides in length, is added to the nucleic acid (e.g., FARs and ssNPPFs) during the second PCR amplification. In a specific example, the poly-A (or poly-T) is added to the 3′-end of the FARs and ssNPPFs. In some examples, this added sequence is polyadenylated at its 3′ end using a terminal deoxynucleotidyl transferase (TdT).

In particular examples, the sequencing adapter added is at least 12 nucleotides (nt) in length, such as at least 15, at least 20, at least 25, at least 30, at least 40, at least 50, at least 60 or at least 70 nt in length, such as 12-50, 20-35, 50-70, 20-70, or 12-30 nt in length.

E. Sequencing Nucleic Acid Amplicons

The resulting nucleic acid amplicons (e.g., FAR amplicons for target DNA and surrogate NPPF amplicons for target RNA) are sequenced, for example, by sequencing the amplicon, or a portion thereof (such as an amount sufficient to permit identification of the target nucleic acid molecule or to permit determination that a particular mutation is or is not present). The disclosure is not limited to a particular sequencing method. It will be appreciated that the nucleic acid amplicons (e.g., DNA amplicons) can be designed for sequencing by any method on any sequencer known currently or in the future. The target nucleic acid itself does not limit the method of sequencing used, nor the sequencing enzyme used. Other methods of sequencing are or will be developed, and one skilled in the art can appreciate that the generated nucleic acid amplicons will be suitable for sequencing on these systems. In some examples, multiple different target nucleic acid amplicons are sequenced in a single reaction. Thus, a plurality of target nucleic acid amplicons can be sequenced in parallel, for example, simultaneously or contemporaneously.

Exemplary sequencing methods that can be used to determine the sequence of the resulting FAR amplicons and NPPF amplicons, such as amplicons composed of DNA, include, but are not limited to, the chain termination method, dye terminator sequencing, and pyrosequencing (such as the methods commercialized by Biotage (for low throughput sequencing) and 454 Life Sciences (for high-throughput sequencing)). In some examples, the amplicons are sequenced using an Illumina® (e.g., NovaSeq, MiSeq), Ion Torrent®, 454®, Helicos, PacBio®, Solid® (Applied Biosystems®) or any other commercial sequencing system. In one example, the sequencing method uses bridge PCR (e.g., Illumina®). In one example, the Helicos® or PacBio® single molecule sequencing method is used. In one example, a next-generation sequencer (NGS) is used, such as those from Illumina®, Roche®, Genapsys, or Thermo Fisher Scientific®, for example, SOLiD®/Ion Torrent® S5 from Thermo Fisher Scientific®, NovaSeq/NextSeq/MiSeq from Illumina®, or GS FLX Titanium®/GS Junior® from Roche®. Sequencing adaptors (such as specific sequences or poly-A or poly T tails present on the FAR amplicons and NPPF amplicons, for example, as introduced using PCR) can be used for capture of the amplicons for sequencing on a particular platform. In one example, a nanopore-type sequencer is used.

Although sequencing by Ion Torrent® or Illumina® typically involves nucleic acid preparation, accomplished by random fragmentation of nucleic acid, followed by in vitro ligation of common adaptor sequences, for the disclosed methods, the step of random fragmentation of the nucleic acid to be sequenced can be eliminated, and the in vitro ligation of adaptor sequences is replaced by sequences present in the NPPF amplicon or FAR amplicon, such as an experiment tag present in the NPPF amplicon or FAR amplicons or a sequencing adaptor sequence present in the NPPF or FAR, or added to the NPPF amplicon or FAR amplicon during amplification. For some sequencing methods, a sequencing primer is hybridized to the amplicons after amplification on the sequencing chip/bead amplicon.

F. Controls

In some examples, the control includes a “positive control” NPPF (e.g., corresponding to a target RNA known to be present in the sample, or to a synthetic target deliberately added to the sample or hybridization reaction) included in the plurality of NPPFs and corresponding CFSs that a sample is contacted with. For example, the corresponding positive control NPPFs and corresponding CFSs can be added to the sample prior to or during hybridization with the plurality of test NPPFs and corresponding CFSs. In some examples, the control includes a “negative control” NPPF (e.g., target RNA known to be absent from the sample) included in the plurality of NPPFs and corresponding CFSs that a sample is contacted with. For example, the corresponding negative control NPPFs and corresponding CFSs can be added to the sample prior to or during hybridization with the plurality of test NPPFs and corresponding CFSs.

In some examples, the control includes a “positive control” NPPF (e.g., target RNA known to be present in the sample) included in the plurality of NPPFs and corresponding CFSs that a sample is contacted with. For example, the corresponding positive control NPPFs and corresponding CFSs can be added to the sample prior to or during hybridization with the plurality of test NPPFs and corresponding CFSs. In some examples, the control includes a “negative control” NPPF (e.g., target RNA known to be absent from the sample) included in the plurality of NPPFs and corresponding CFSs that a sample is contacted with. For example, the corresponding negative control NPPFs and corresponding CFSs can be added to the sample prior to or during hybridization with the plurality of test NPPFs and corresponding CFSs.

In some examples, the control includes a “positive control” DNA (e.g., target DNA known to be present in the sample) and corresponding primers included in the portion of the lysed sample where DNA is amplified. For example, the corresponding positive control DNA and corresponding primers and can be added to the sample prior to or during hybridization with the target DNA amplification primers (e.g., step 2A of FIG. 3). In some examples, the control includes a “negative control” DNA (e.g., target DNA known to be absent from the sample) included in the portion of the lysed sample where DNA is amplified. For example, the corresponding negative control DNA and primers can be added to the sample prior to or during hybridization with the target DNA amplification primers (e.g., step 2A of FIG. 3).

In some examples, this positive control is an internal normalization control for variables such as the number of cells lysed for each sample, recovery of RNA or DNA, hybridization efficiency, or error introduced by amplification and sequencing. In some examples the positive control includes one or more NPPFs and corresponding CFSs specific for an RNA known to be present in the sample (for example a nucleic acid sequence likely to be present in the species being tested, such as one or more basal level or constitutive housekeeping RNAs). Exemplary DNA positive control targets include, but are not limited to, structural genes (e.g., actin, tubulin, or others) or DNA binding proteins (e.g., transcription regulation factors, or others), as well as housekeeping genes.

In some examples, a positive control target includes one or more NPPFs and corresponding CFSs specific for RNA from glyceraldehyde-3-phosphate dehydrogenase (GAPDH), peptidylproylyl isomerase A (PPIA), large ribosomal protein (RPLPO), ribosomal protein L19 (RPL19), SDHA (succinate dehydrogenase), HPRT1 (hypoxanthine phosphoribosyl transferase 1), HBS1L (HBS1-like protein), 3-actin (ACTB), 5-Aminolevulinic acid synthase 1 (ALAS1), β-2 microglobulin (B2M), alpha hemoglobin stabilizing protein (AHSP), ribosomal protein S13 (RPS13), ribosomal protein S20 (RPS20), ribosomal protein L27 (RPL27), ribosomal protein L37 (RPL37), ribosomal protein 38 (RPL38), ornithine decarboxylase antizyme 1 (OAZ1), polymerase (RNA) II (DNA directed) polypeptide A, 220 kDa (POLR2A), thioredoxin like 1 (TXNL1), yes-associated protein 1 (YAP1), esterase D (ESD), proteasome (prosome, macropain) 26S subunit, ATPase, 1 (PSMC1), eukaryotic translation initiation factor 3, subunit A (EIF3A), or 18S rRNA. In some examples, a positive control target includes one or more of these DNA molecules (or a portion thereof). In some examples, the positive control targets are repetitive DNA elements such as HSAT1, ACRO1, and LTR3. In some examples, the positive control targets are single-copy genomic DNA sequences (assuming a haploid genome).

In some examples, a positive control includes one or more NPPFs and corresponding CFSs, whose complement is a spiked in (e.g., added) target nucleic acid molecule (such as one or more in vitro transcribed nucleic acids, nucleic acids isolated from an unrelated sample, or synthetic nucleic acids such as a DNA or RNA oligonucleotide) added to the sample prior to or during hybridization with the plurality of NPPFs and corresponding CFSs. In one example, the positive control NPPFs and spike ins have a single nucleotide mismatch. In one example, a plurality of NPPFs and spike ins are added, with the spike ins added at a range of known concentrations (such as 1 pM, 10 pM, and 100 pM) that form a “ladder” of input and demonstrate the dynamic range of the assay in the final sequencing output.

In some examples, a “negative control” includes one or more NPPFs and corresponding CFSs, whose complement is known to be absent from the sample, for example as a control for hybridization specificity, such as a nucleic acid sequence from a species other than that being tested, e.g., a plant nucleic acid sequence when human nucleic acids are being analyzed (for example, Arabidopsis thaliana AP2-like ethylene-responsive transcription factor (ANT)), or a nucleic acid sequence not found in nature. In some examples, a “negative control” includes one or more DNAs (and corresponding primers), known to be absent from the sample, such as DNA from a species other than that being tested, e.g., a plant nucleic acid sequence when human nucleic acids are being analyzed (for example, Arabidopsis thaliana AP2-like ethylene-responsive transcription factor (ANT)), or a nucleic acid sequence not found in nature.

In some examples, the control is used to determine if a particular step in the method is operating properly. In some examples, the positive or negative controls are assessed in the final sequencing results. In one such example, this analysis includes the use of Taqman or other detectable qPCR probes for the negative control probes to assess the effectiveness of the nuclease. All negative control NPPF should be removed by the nuclease step, therefore if the amount of negative control NPPF is high, it may indicate that the nuclease protection did not perform properly and that the sample may be compromised. In another such example, the Taqman assay for negative control probes is combined with a simultaneous measurement quantification of the amount of the entire captured target (i.e., using SYBR-based qPCR methods).

In one example, the sample to be analyzed is exposed to amplification conditions (e.g., qPCR) prior to performing the disclosed methods, to determine if the sample has a sufficient amount of (and quality of) nucleic acid molecules. For example, qPCR may be performed using primers that amplify a target region of interest such as KRAS or BRAF, a housekeeper RNA gene such as GAPDH, or a repetitive DNA element such as LTR3 to determine the assessable nucleic acid within the sample. In one example, the primers are designed such that they amplify a region close to the size of the target region, to determine whether available nucleic acid is large enough to be assessed. In one example, the range of acceptable sample amounts and qualities is determined experimentally, for example using a particular sample type (e.g., lung or melanoma samples) or format (e.g., formalin fixed tissues or cell lines).

### III. Nuclease Protection Probes with Flanking Sequences (NPPFs)

The disclosed methods permit sequencing of DNA and RNA in the same sample, in part by using a surrogate for the RNA, namely an NPPF. The NPPF amplicons and FAR amplicons can be sequenced from the same mixture simultaneously or contemporaneously. Based on the target RNA, NPPFs can be designed for use in the disclosed methods using the criteria set forth herein in combination with the knowledge of one skilled in the art. In some examples, the disclosed methods include generation of one or more appropriate NPPFs for detection of particular target RNA molecules. The NPPF, under a variety of conditions (known or empirically determined), specifically binds (or is capable of specifically binding, e.g., specifically hybridizing) to a target RNA or portion thereof, if such target RNA is present in the sample.

FIG. 1A shows an exemplary NPPF 100 having a region 102 that includes a sequence that specifically binds to or hybridizes to the target nucleic acid sequence(s), as well as flanking sequences 104, 106 at the 5′- and 3′-end of the NPPF, respectively, wherein the flanking sequences bind or hybridize to their complementary sequences (referred to herein as CFSs). Although two flanking sequences are shown, in some examples the NPPF has only one flanking sequence, such as one at the 5′-end or one at the 3′-end. In some examples, the NPPF includes two flanking sequences: one at the 5′-end and the other at the 3′-end. In some examples, the flanking sequence at the 5′-end differs from the flanking sequence at the 3′-end. FIG. 1B shows an embodiment of an NPPF 120 that is composed of two separate nucleic acid molecules 128, 130. In one example, the NPPF is 100 nt, 25 nt for each flanking sequence 104, 106, and 50 nt for the region 102 that specifically binds to or hybridizes to the target nucleic acid sequence(s).

The NPPF (as well as CFSs that bind to the NPPFs) can be any nucleic acid molecule, such as a DNA or RNA molecule, and can include unnatural bases. Thus, the NPPFs (as well as CFSs that bind to the NPPFs) can be composed of natural (such as ribonucleotides (RNA), or deoxyribonucleotides (DNA)) or unnatural nucleotides (such as locked nucleic acids (LNAs, see, e.g., U.S. Pat. No. 6,794,499), peptide nucleic acids (PNAs)), and the like. The NPPFs can be single- or double-stranded. In one example, the NPPFs and CFSs are ssDNA. In one example, the NPPF is a ss DNA and the CFS(s) is/are RNA (e.g., and the target is RNA). In some examples, the NPPFs (as well as CFSs that bind to the NPPFs) include one or more synthetic bases or alternative bases (such as inosine). Modified nucleotides, unnatural nucleotides, synthetic, or alternative nucleotides can be used in NPPFs at one or more positions (such as 1, 2, 3, 4, 5, or more positions). For example, NPPFs and/or CFSs can include one or more nucleotides containing modified bases, and/or modified phosphate backbones. In some examples, use of one or more modified or unnatural nucleotides in the NPPF can increase the Tₘ of the NPPF relative to the Tₘ of a NPPF of the same length and composition which does not include the modified nucleic acid. One of skill in the art can design probes including such modified nucleotides to obtain a probe with a desired Tₘ. In one example, an NPPF is composed of DNA or RNA, such as single stranded (ssDNA) or branched DNA (bDNA). In one example, an NPPF is an aptamer.

The NPPFs include a region that is complementary to one or more target RNA molecules. NPPFs used in the same reaction can be designed to have similar Tₘ's. In one example, at least one NPPF is present in the reaction that is specific for a single target RNA sequence. In such an example, if there are 2, 3, 4, 5, 6, 7, 8, 9 or 10 different target RNA sequences to be detected or sequenced using NPPFs as surrogates, the method can correspondingly use at least 2, 3, 4, 5, 6, 7, 8, 9 or 10 different NPPFs (wherein each NPPF corresponds to/has sufficient complementarity to hybridize to a particular RNA target). Thus in some examples, the methods use at least two NPPFs, wherein each NPPF is specific for a different target RNA molecule. However, one will appreciate that several different NPPFs can be generated to a particular target RNA molecule, such as many different regions of a single target RNA sequence.

However, in some examples, a single NPPF is present in the reaction is specific for two or more target RNA sequences, such as a wild type RNA sequence and one or more alternative sequences for a particular RNA. Thus, in some examples, a single NPPF is present in the reaction is specific for two or more target RNA sequences, such as a wild type RNA sequence and one or more mutant sequences or one or more different splice isoforms for a particular RNA (such as 2-15 different transcripts from the same RNA). For example, if there are 2, 3, 4, 5, 6, 7, 8, 9, 10, 11, 12, 13, 14, or 15 different RNA isoforms, one skilled in the art will appreciate that an NPPF can be designed to only hybridize to one splice isoform, such that the NPPF hybridizes over a splice junction or in a region of sequence unique to that isoform.

Combinations of NPPFs can be used in a single reaction, such as (1) one or more NPPFs each having specificity (e.g., complementarity) for a single target RNA sequence (e.g., can only sufficiently hybridize to a single target RNA molecule), and (2) one or more NPPFs each having specificity (e.g., complementarity) for a single target RNA, but with the ability to detect a plurality of variations in that RNA (e.g., can sufficiently hybridize to two or more variations of the target RNA, such as the wild type sequence and at least one splice isoform, such as 2, 3, 4, 5, 6, 7, 8, 9, 10, 11, 12, 13, 14 or 15 different transcripts of the wild type RNA sequence). In some examples, the reaction includes (1) at least 2, at least 3, at least 4, at least 5, at least 10, at least 15, at least 20, at least 25, or at least 30 different NPPFs that each have specificity (e.g., complementarity) for a single target RNA sequence, and (2) at least 2, at least 3, at least 4, at least 5, at least 10, at least 15, at least 20, at least 25, or at least 30 different NPPFs each having specificity (e.g., complementarity) for a single target RNA, but with the ability to detect a plurality of variations in that RNA.

Thus, at least one portion (such as a second portion) of a single sample may be contacted with one or more NPPFs. A set of NPPFs is a collection of two or more NPPFs each specific for (1) a different target RNA sequence and/or a different portion of a same target RNA, or specific for (2) a single target RNA but with the ability to detect variations of the RNA sequence. A set of NPPFs can include at least, up to, or exactly 2, 3, 4, 5, 6, 7, 8, 9, 10, 12, 15, 20, 25, 30, 50, 100, 500, 1000, 2000, 3000, 5000, 10,000, 15,000, 20,000, 25,000, 30,000, 35,000, 40,000 or 50,000 different NPPFs. In some examples, at least one portion (such as a second portion) of a sample is contacted with a sufficient amount of NPPF to be in excess of the target(s) for such NPPF, such as a 100-fold, 500-fold, 1000-fold, 10,000-fold, 100,000-fold or 10⁶-fold excess. In some examples, if a set of NPPFs is used, each NPPF of the set can be provided in excess to its respective target(s) (or portion of a target(s)) in the at least one portion (such as a second portion) of the sample. Excess NPPF can facilitate quantitation of the amount of NPPF that binds a particular target(s). Some method embodiments involve a plurality of samples (e.g., at least, up to, or exactly 10, 25, 50, 75, 100, 500, 1000, 2000, 3000, 5000 or 10,000 different samples) with at least one portion (such as a second portion) thereof simultaneously or contemporaneously contacted with the same NPPF or set of NPPFs.

Methods of empirically determining the appropriate size of a NPPF for use with a particular target(s) or samples (such as fixed or crosslinked samples) are routine. In specific embodiments, a NPPF can be up to 500 nucleotides in length, such as up to 400, up to 250, up to 100, or up to 75 nucleotides in length, including, for example, in the range of 20 to 1500, 20 to 1250, 25 to 1200, 25 to 1100, 25-75, 25 to 150, 75 to 100, 90 to 110, 100 to 250, or 125 to 200 nt in length. In one non-limiting example, an NPPF is at least 35 nt in length, such as at least 40, at least 45, at least 50, at least 75, at least 100, at least 150, at least 180, or at least 200 nt in length, such as 50 to 200, 50 to 150, 50 to 100, 75 to 200, 40 to 80, 35 to 150, or 36, 72, 75, 100, 125, 150, 160, 170, 180, 190, or 200 nt in length. In one example, the RNA target is mRNA and the NPPF is 100 nt. In one example, the RNA target is miRNA, and the NPPF is 75 nt. Particular NPPF embodiments may be longer or shorter depending on desired functionality. In some examples, the NPPF is appropriately sized (e.g., sufficiently small) to penetrate fixed and/or crosslinked samples. Fixed or crosslinked samples may vary in the degree of fixation or crosslinking; thus, an ordinarily skilled artisan may determine an appropriate NPPF size for a particular sample condition or type, for example, by running a series of experiments using samples with known, fixed target concentration(s) and comparing NPPF size to target signal intensity. In some examples, the sample (and, therefore, at least a proportion of target) is fixed or crosslinked, and the NPPF is sufficiently small that signal intensity remains high and does not substantially vary as a function NPPF size.

Factors that affect NPPF-target and NPPF-CFS hybridization specificity include length of the NPPF and CFS, melting temperature, self-complementarity, and the presence of repetitive or non-unique sequence. See, e.g., Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999. Conditions resulting in particular degrees of hybridization (stringency) will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. In some examples, the NPPFs utilized in the disclosed methods have a Tₘ of at least about 37° C., at least about 42° C., at least about 45° C., at least about 50° C., at least about 55° C., at least about 60° C., at least about 65° C., at least about 70° C., at least about 75° C., at least about 80° C., such as about 42° C.-80° C. (for example, about 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C.). In one non-limiting example, the NPPFs utilized in the disclosed methods have a Tₘ of about 42° C. Methods of calculating the Tₘ of a probe are known to one of skill in the art (see e.g., Sambrook et al., Molecular Cloning: A Laboratory Manual, 3d ed., Cold Spring Harbor Press, 2001, Chapter 10). In some examples, the NPPFs for a particular reaction are selected to each have the same or a similar Tₘ in order to facilitate simultaneous detection or sequencing of multiple target nucleic acid molecules in a sample, such as Tₘs+/− about 10° C. of one another, such as +/−10° C., 9° C., 8° C., 7° C., 6° C., 5° C., 4° C., 3° C., 2° C., or 1° C. of one another.

A. Region that Hybridizes to the Target

The portion of the NPPF sequence (shown in FIG. 1) 102 (or 122) that specifically hybridizes to a target RNA is complementary in sequence to the target RNA sequence(s) of interest. This complementarity can be designed such that the NPP only hybridizes to a single target RNA sequence or can hybridize to a plurality of target RNA sequences, such as wild type RNA and variations thereof.

One skilled in the art will appreciate that the sequence 102 (or 122) need not be complementary to an entire target RNA (e.g., if the target is a gene of 100,000 nucleotides, the sequence 102 (or 122) can be a portion of that, such as at least 10, at least 15, at least 20, at least 25, at least 30, at least 40, at least 50, at least 100, or more consecutive nucleotides complementary to a particular target RNA molecule(s)). The specificity of a probe increases with length. Thus for example, a sequence 102 (or 122) that specifically binds to the target RNA sequence(s) which includes 25 consecutive ribonucleotides will anneal to a target sequence with a higher specificity than a corresponding sequence of only 15 ribonucleotides. Thus, the NPPFs disclosed herein can have a sequence 102 (or 122) that specifically binds to the target RNA sequence(s) which includes at least 6, at least 10, at least 15, at least 20, at least 25, at least 30, at least 40, at least 50, at least 60, at least 100, or more consecutive nucleotides complementary to a particular target RNA molecule (such as about 6 to 50, 6 to 60, 10 to 40, 10 to 60, 15 to 30, 15 to 27, 16 to 27, 16 to 50, 15 to 50, 18 to 23, 19 to 22, or 20 to 25 consecutive nucleotides complementary to a target RNA).

Particular lengths of sequence 102 (or 122) that specifically binds to the target RNA sequence(s) that can be part of the NPPFs used to practice the methods of the present disclosure include 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 contiguous nucleotides complementary to a target RNA molecule. In one example, the length of the sequence 102 (or 122) that specifically binds to the target RNA is 50 nt. In some examples where the target RNA molecule is an miRNA (or siRNA), the length of the sequence 102 (or 122) that specifically binds to the target RNA sequence can be shorter, such as 16 to 27 nucleotides in length (such as 16, 17, 18, 19, 20, 21, 22, 23, 24, 25, 26, or 27 nt) to match the miRNA (or siRNA) length. However, one skilled in the art will appreciate that the sequence 102 (or 122) that specifically binds to the target RNA need not be 100% complementary to the target RNA molecule. In some examples, the region of the NPPF complementary to the target and the target RNA share at least 80%, at least 90%, at least 95%, at least 96%, at least 97%, at least 98%, at least 99%, or 100% complementarity, but wherein any mismatch can survive digestion with a nuclease.

B. Flanking Sequence(s)

The sequence of the flanking sequence 104, 106 (or 124, 126) provides a complementary sequence to which CFSs can specifically hybridize (similarly, the sequence of flanking sequence 238, 239 in FIG. 3 has a complementary sequence to which amplification primers in the second amplification step can hybridize). Thus, each flanking sequence 104, 106 (or 124, 126) is complementary to at least a portion of a CFS (e.g., a 5′-flanking sequence is complementary to a 5CFS and a 3′-flanking sequence is complementary to a 3CFS). The flanking sequence is not similar to a sequence otherwise found in the sample (e.g., not found in the human genome). Thus, the flanking sequence includes a sequence of contiguous nucleotides not found in a nucleic acid molecule otherwise present in the sample. For example, if the target nucleic acid is a human sequence, the sequence of the flanking sequence is not similar to a sequence found in the target (e.g., human) genome. This helps to reduce non-specific binding (or cross-reactivity) of non-target sequences that may be present in the target genome to the NPPFs. Methods of analyzing a sequence for its similarity to a genome are known.

An NPPF can include one or two flanking sequences (e.g., one at the 5′-end, one at the 3′-end, or both), and the flanking sequences can be the same or different. In specific examples, each flanking sequence does not specifically bind to any other NPPF sequence (e.g., sequence 102, 122 or other flanking sequence) or to any component of the sample. In some examples, if there are two flanking sequences, the sequence of each flanking sequence 104, 106 (or 124, 126) is different. If there are two different flanking sequences (for example two different flanking sequences on the same NPPF and/or to flanking sequences of other NPPFs in a set of NPPFs), each flanking sequence 104, 106 (or 124, 126) in some examples has a similar melting temperature (Tₘ), such as a Tₘ+/− about 10° C. or +/−5° C. of one another, such as +/−4° C., 3° C., 2° C., or 1° C.

In one example, the flanking sequence 104, 106 (or 124, 126) portion of the NPPF includes at least one nucleotide mismatch. That is, at least one nucleotide is not complementary to its corresponding nucleotide in the CFS, and thus will not form a base pair at this position.

The flanking sequence(s) of the NPPF (and the FAR) can provide a universal amplification point that is complementary to at least a portion of an amplification primer used in Step 4 of FIG. 3. Thus, the flanking sequence(s) permit use of the same amplification primers to amplify surrogate NPPFs specific for different target RNA molecules and to amplify the FARs for target DNA molecules. Thus, at least a portion of sequence of the flanking sequence(s) can be complimentary to at least a portion of an amplification primer used in the second amplification reaction. As shown in FIG. 4, this allows the primer to hybridize to the flanking sequence(s), and amplify the ssNPPF for the target RNA and the FAR for the target DNA. As flanking sequence(s) can be identical between NPPFs (and the FARs), while the region specific for different target nucleic acid molecules and vary, this permits the same primer to be used to amplify (1) any number of different ssNPPFs for different RNA targets and (2) any number of different FARs for different DNA targets, in the same reaction (e.g., co-amplify both the different ssNPPFs and different FARs). Thus an amplification primer that includes a sequence complementary to the 5′ flanking sequence(s), and an amplification primer that includes a sequence complementary to the 3′ flanking sequence(s), can both be used in a single reaction to amplify NPPFs and FARs, even if the NPPF target RNA sequences differ and the FAR target DNA sequences differ.

In some examples, the flanking sequence(s) do not include an experiment tag sequence and/or a sequencing adaptor sequence. In some examples, flanking sequence(s) include or consist of an experiment tag sequence and/or sequencing adaptor sequence. In other examples, the primers used to amplify the ssNPPFs and FARs (which include at least one flanking sequence) include an experiment tag sequence and/or sequencing adaptor sequence (such as a poly-A or poly-T sequence needed for some sequencing platforms), thus, permitting incorporation of the experiment tag and/or sequencing adaptor into the NPPF amplicon and FAR amplicon during amplification of NPPF (step 4 in FIG. 3). Experimental tags and sequencing adaptors are described above in Section II, D. One will appreciate that more than one experiment tag can be included (such as at least 2, at least 3, at least 4, or at least 5 different experiment tags), such as those used to uniquely identify a target DNA or RNA, or identify a sample.

In particular examples, the flanking sequence 104, 106 (or 124, 126) portion of the NPPF (or FAR) is at least 12 nucleotides in length, or at least 25 nucleotides in length, such as at least 15, at least 20, at least 25, at least 30, at least 40, or at least 50 nucleotides in length, such as 12 to 50, 12 to 25, or 12 to 30 nucleotides, for example, 20, 21, 22, 23, 24, 25, 26, 27, 28, 29 or 30 nucleotides in length, wherein the contiguous nucleotides are not found in a nucleic acid molecule present in the sample to be tested. In one example, the flanking sequence 104, 106 (or 124, 126) portion of the NPPF (or FAR) is 25 nt in length. The flanking sequences are protected from degradation by the nuclease by hybridizing molecules to the flanking sequences which have a sequence complementary to the flanking sequences (CFSs).

### IV. Complementary Flanking Sequences (CFSs)

Each CFS (e.g., 208 or 210 of FIG. 3) is complimentary to its corresponding flanking sequence of the NPPF. The method can use at least one CFS. For example, the method can use a single CFS (with an NPPF having one flanking sequence) or two CFSs (with an NPPF having two flanking sequences), one at the 5′-end, the other at the 3′-end of the target RNA. For example, if an NPPF includes a 5′-flanking sequence, a 5CFS will be used in the method. If an NPPF includes a 3′-flanking sequence, a 3CFS will be used in the method. If the 5′- and the 3′-flanking sequences are different from one another, the 5CFS and 3CFS will be different from one another. One skilled in the art will appreciate that the CFS and the flanking sequence of the NPPF need not be 100% complementary (i.e., need not have 100% complementarity), as long as hybridization can occur between the NPPF and its RNA target and corresponding CFS(s). In some examples, the flanking sequence of the NPPF and the CFS share at least 80%, at least 90%, at least 95%, at least 98%, at least 99%, or 100% complementarity. In some examples the CFS is the same length as its corresponding flanking sequence of the NPPF. For example, if the flanking sequences 25 nt, the CFS can be 25 nt.

In some examples, the CFS is not similar to a sequence found in the target genome. For example, if the target RNA is a human sequence, the sequence of the CFS (and corresponding flanking sequence) is not similar to a RNA sequence found in the target genome. This helps to reduce binding of non-target sequences that may be present in the target genome from binding to the CFSs (and NPPFs). Methods of analyzing a sequence for its similarity to a genome are known.

### V. Samples

A sample is any collective comprising one or more targets, such as a biological sample or biological specimen, such as those obtained from a subject (such as a human or other mammalian subject, such as a veterinary subjects, for example a subject known or suspected of having a tumor or an infection). The sample can be collected or obtained using methods known to those ordinarily skilled in the art. The samples of use in the disclosed methods can include any specimen that includes nucleic acid (such as genomic DNA, cDNA, viral DNA or RNA, rRNA, tRNA, mRNA, miRNA, oligonucleotides, nucleic acid fragments, modified nucleic acids, synthetic nucleic acids, or the like). In one example, the sample includes RNA and DNA. In some examples, the target nucleic acid molecule to be sequenced is cross-linked in the sample (such as a cross-linked DNA, mRNA, miRNA, or vRNA) or is soluble in the sample. In some examples, the sample is a fixed sample, such as a sample that includes an agent that causes target molecule cross-linking (and thus in some examples the target nucleic acid molecule can be fixed). In some examples, the target nucleic acids in the sample are not extracted, solubilized, or both, prior to detecting or sequencing the target nucleic acid molecule (or a surrogate thereof). In some examples, the sample is an ex situ biological sample.

In some examples, the disclosed methods include obtaining the sample prior to analyzing the sample. In some examples, the disclosed methods include selecting a subject having a particular disease or tumor, and then in some examples further selecting one or more target DNAs and one or more RNAs to detect based on the subject's particular disease or tumor, for example, to determine a diagnosis or prognosis for the subject or for selection of one or more therapies. In some examples, nucleic acid molecules in a sample to be analyzed are first isolated, extracted, concentrated, or combinations thereof, from the sample. In some examples, nucleic acid molecules in a sample to be analyzed are not isolated, extracted, concentrated, or combinations thereof, from the sample, prior to their analysis.

In some examples, reference to “a” or “the” sample refers to one single or individual sample, such as one slice or section from an FFPE tissue block. In some examples, a single or individual sample analyzed using the disclosed methods has less than 250,000 cells (for example less than 100,000, less than 50,000, less than 10,000, less than 1,000, less than 500, less than 200, less than 100 cells, or less than 10 cells, such as 1 to 250,000 cells, 1 to 100,000 cells, 1 to 10,000 cells, 1 to 1000 cells, 1 to 100 cells, 1 to 50 cells, 1 to 25 cells, or about 1 cell). In some examples, two or more single or individual samples are analyzed simultaneously (but in some examples separately) using the disclosed methods, for example where each single or individual sample is different, for example from different subjects, from different tissues, or from different parts of the same tissue.

In some examples, the sample, such as an ex situ sample, is lysed. The lysis buffer in certain examples may inactivate enzymes that degrade RNA, but a limited dilution into a hybridization dilution buffer permits nuclease activity and facilitates hybridization with stringent specificity. A dilution buffer can be added to neutralize the inhibitory activity of the lysis and other buffers, such as inhibitory activity for other enzymes (e.g., polymerase). Alternatively, the composition of the lysis buffer and other buffers can be changed to a composition that is tolerated, for example by a polymerase or ligase.

In some examples, the methods include analyzing a plurality of samples simultaneously or contemporaneously. For example, the methods can analyze at least two different samples (for example from different subjects, e.g., patients) simultaneously or contemporaneously. In one such example, the methods further can detect or sequence at least two different target DNA and at least two different RNA molecules (such as 2, 3, 4, 5, 6, 7, 8, 9 or 10 different targets) in at least two different samples (such as at least 5, at least 10, at least 100, at least 500, at least 1000, or at least 10,000 different samples) simultaneously or contemporaneously.

Exemplary samples include, without limitation, cells, cell lysates, blood smears, cytocentrifuge preparations, flow-sorted or otherwise selected cell populations, cytology smears, chromosomal preparations, bodily fluids (e.g., blood and fractions thereof such as white blood cells, serum or plasma; saliva; sputum; urine; spinal fluid; gastric fluid; sweat; semen; nipple aspirate fluid (NAF), etc.), buccal cells, extracts of tissues, cells or organs, tissue biopsies (e.g., tumor or lymph node biopsies), liquid biopsies, fine-needle aspirates, bronchoscopic lavage, punch biopsies, circulating tumor cells, extracellular vesicles, circulating nucleic acids from tumors, bone marrow, amniocentesis samples, autopsy material, fresh tissue, frozen tissue, fixed tissue, fixed and wax- (e.g., paraffin-) embedded tissue, bone marrow, and/or tissue sections (e.g., cryostat tissue sections and/or paraffin-embedded tissue sections). The biological sample may also be a laboratory research sample such as a cell culture sample or supernatant. In one example, the sample analyzed is a single section of FFPE tissue about five microns thick.

Exemplary samples may be obtained from normal cells or tissues, or from neoplastic cells or tissues. Neoplasia is a biological condition in which one or more cells have undergone characteristic anaplasia with loss of differentiation, increased rate of growth, invasion of surrounding tissue, and in which cells may be capable of metastasis. In particular examples, a biological sample includes a tumor sample, such as a sample containing neoplastic cells.

Exemplary neoplastic cells or tissues may be included in or isolated from solid tumors, including lung cancer (e.g., non-small cell lung cancer, such as lung squamous cell carcinoma), breast carcinomas (e.g., lobular and ductal carcinomas), adrenocortical cancer, ameloblastoma, ampullary cancer, bladder cancer, bone cancer, cervical cancer, cholangioma, colorectal cancer, endometrial cancer, esophageal cancer, gastric cancer, glioma, granular cell tumors, head and neck cancer, hepatocellular cancer, hydatiform mole, lymphoma, melanoma, mesothelioma, myeloma, neuroblastoma, oral cancer, osteochondroma, osteosarcoma, ovarian cancer, pancreatic cancer, pilomatricoma, prostate cancer, renal cell cancer, salivary gland tumor, soft tissue tumors, Spitz nevus, squamous cell cancer, teratoid cancer, and thyroid cancer. Exemplary neoplastic cells may also be included in or isolated from hematological cancers including leukemias, including acute leukemias (such as acute lymphocytic leukemia, acute myelocytic leukemia, acute myelogenous leukemia, erythroleukemia, and myeloblastic, promyelocytic, myelomonocytic, and monocytic leukemias), chronic leukemias (such as chronic myelocytic (granulocytic) leukemia, chronic myelogenous leukemia, and chronic lymphocytic leukemia), polycythemia vera, lymphomas such as Hodgkin's disease or non-Hodgkin's lymphoma (indolent and high grade forms), multiple myeloma, Waldenstrom's macroglobulinemia, heavy chain disease, myelodysplastic syndrome, and myelodysplasia.

For example, a sample from a tumor that contains cellular material can be obtained by surgical excision of all or part of the tumor, by biopsy techniques such as needle biopsies, by collecting a fine needle aspirate from the tumor, as well as other methods. In some examples, a tissue or cell sample is applied to a substrate and analyzed to determine the presence of one or more target DNAs and one or more target RNAs. A solid support useful in a disclosed method need only bear the biological sample and, optionally, permit the convenient detection of components (e.g., proteins and/or nucleic acid sequences) in the sample. Exemplary supports include microscope slides (e.g., glass microscope slides or plastic microscope slides), coverslips (e.g., glass coverslips or plastic coverslips), tissue culture dishes, multi-well plates, membranes (e.g., nitrocellulose or polyvinylidene fluoride (PVDF)) or BIACORE™ chips.

The disclosed methods are sensitive and specific and allow sequencing of target nucleic acid molecules in a sample containing even a limited number of cells. Samples that include small numbers of cells, such as less than 250,000 cells (for example less than 100,000, less than 50,000, less than 10,000, less than 1,000, less than 500, less than 200, less than 100 cells, or less than 10 cells, include but are not limited to, FFPE samples, fine needle aspirates (such as those from lung, prostate, lymph, breast, or liver), punch biopsies, needle biopsies, bone marrow biopsies, small populations of (e.g., FACS) sorted cells or circulating tumor cells, lung aspirates, small numbers of laser captured, flow-sorted, or macrodissected cells or circulating tumor cells, exosomes and other subcellular particles, or body fluids (such as plasma, serum, spinal fluid, saliva, semen, and breast aspirates). For example, a target DNA and target RNA (e.g, via a surrogate) can be sequenced (and thus detected) in as few as 100 cells (such as a sample including 100 or more cells, such as 100, 500, 1000, 2000, 3000, 4000, 5000, 6000, 7000, 8000, 9000, 10,000, 15,000, 20,000, 50,000, or more cells). In some examples, expression of a target DNA and target RNA can be detected in about 1000 to 100,000 cells, for example about 1000 to 50,000, 1000 to 15,000, 1000 to 10,000, 1000 to 5000, 3000 to 50,000, 6000 to 30,000, or 10,000 to 50,000 cells). In some examples, expression of a target DNA and target RNA can be detected in about 100 to 250,000 cells, for example about 100 to 100,000, 100 to 50,000, 100 to 10,000, 100 to 5000, 100 to 500, 100 to 200, or 100 to 150 cells. In other examples, a target DNA and target RNA (e.g, via a surrogate) can be sequenced in about 1 to 1000 cells (such as about 1 to 500 cells, about 1 to 250 cells, about 1 to 100 cells, about 1 to 50 cells, about 1 to 25 cells, or about 1 cell).

Samples may be treated in a number of ways prior to (or contemporaneous with) contacting the sample with a target-specific reagent (such as NPPFs and corresponding CFSs for target RNA, or with primers for target DNA). One relatively simple treatment is suspension of the sample in a buffer, e.g., lysis buffer, which conserves all components of the sample in a single solution. In some examples, the sample is treated to partially or completely isolate (e.g., extract) a target (e.g., DNA and mRNA) from the sample. A target (such as DNA and RNA) has been isolated or extracted when it is purified away from other non-target biological components in a sample. Purification refers to separating the target from one or more extraneous components (e.g., organelles, proteins) also found in a sample. Components that are isolated, extracted or purified from a mixed specimen or sample typically are enriched by at least 50%, at least 60%, at least 75%, at least 90%, or at least 98% or even at least 99% compared to the unpurified or non-extracted sample.

Isolation of biological components from a sample is time consuming and bears the risk of loss of the component that is being isolated, e.g., by degradation and/or poor efficiency or incompleteness of the process(es) used for isolation. Moreover, with some samples, such as fixed tissues, targets (such as DNA and RNA (e.g., mRNA or miRNA)) are notoriously difficult to isolate with high fidelity (e.g., as compared to fresh or frozen tissues) because it is thought that at least some proportion of the targets are cross-linked to other components in the fixed sample and, therefore, cannot be readily isolated or solubilized and may be lost upon separation of soluble and insoluble fractions. Additionally, isolated DNA and RNA from fixed samples is often fragmented into short pieces. Very short DNA and RNA fragments may be lost during precipitation or matrix-binding steps, leading to measurement biases. Accordingly, in some examples, the disclosed methods of sequencing a target nucleic acid do not require or involve purification, extraction or isolation of a target nucleic acid molecules from a sample prior to contacting the lysed sample with amplification primers or NPPF(s) and corresponding CFS(s), and/or involve only suspending the sample in a solution, e.g., lysis buffer, that retains all components of the sample prior to contacting the sample with amplification primers or NPPF(s) and corresponding CFS(s). Thus, in some examples, the methods do not include isolating nucleic acid molecules from a sample prior to their analysis.

In some examples, cells in the sample are lysed or permeabilized in an aqueous solution (for example using a lysis buffer). The aqueous solution or lysis buffer includes detergent (such as sodium dodecyl sulfate) and one or more chaotropic agents (such as formamide, guanidinium HCl, guanidinium isothiocyanate, or urea). The solution may also contain a buffer (for example SSC). In some examples, the lysis buffer includes about 8% to 60% formamide (v/v) about 0.01% to 0.1% SDS, and about 0.5-6×SSC (for example, about 3×SSC). The buffer may optionally include tRNA (for example, about 0.001 to about 2 mg/ml); a ribonuclease; DNase; proteinase K; enzymes (e.g. collagenase or lipase) that degrade protein, matrix, carbohydrate, lipids, or one species of oligonucleotides, or combinations thereof. The lysis buffer may also include a pH indicator, such as phenol red. Cells are incubated in the aqueous solution (optionally overlaid with oil) for a sufficient period of time (such as about 1 minute to about 6 hours, for example about 30 minutes to 3 hours, about 2 to 6 hours, about 3 to 6 hours, about 5 minutes to about 20 minutes, or about 10 minutes) and at a sufficient temperature (such as about 22° C. to about 110° C., for example, about 80° C. to about 105° C., about 37° C. to about 105° C., or about 90° C. to about 100° C.) to lyse or permeabilize the cell. In some examples, lysis is performed at about 50° C., 65° C., 95° C., or 105° C.

In one example, the sample is an FFPE sample (such as an FFPE slice or RNA and DNA isolated from such a sample), and the cells are lysed for at least 2 hours, such as at least 3 hours, at least 4 hours, at least 5 hours, or at last 6 hours, for example at 50° C. following a brief period at 95° C. or 105° C. In one example Proteinase K is included with the lysis buffer.

In some examples, the crude cell lysis is used directly without further purification. The crude cell lysis can be divided into one or more portions, such as portions of equal volume, wherein one or more of the NPPFs and corresponding CFSs are added to at least one first portion, and one or more amplification primers are added to a different/second portion. In other examples, nucleic acids (such as DNA and RNA) are isolated from the cell lysate prior to contacting the lysate with one or more NPPFs and corresponding CFSs or with the amplification primers.

In other examples, tissue samples are prepared by fixing and embedding the tissue in a medium or include a cell suspension is prepared as a monolayer on a solid support (such as a glass slide), for example by smearing or centrifuging cells onto the solid support. In further examples, fresh frozen (for example, unfixed) tissue or tissue sections may be used in the methods disclosed herein. In particular examples, FFPE tissue sections are used in the disclosed methods.

In some examples an embedding medium is used. An embedding medium is an inert material in which tissues and/or cells are embedded to help preserve them for future analysis. Embedding also enables tissue samples to be sliced into thin sections. Embedding media include paraffin, celloidin, OCT™ compound, agar, plastics, or acrylics. Many embedding media are hydrophobic; therefore, the inert material may need to be removed prior to analysis, which utilizes primarily hydrophilic reagents. The term deparaffinization or dewaxing refers to the partial or complete removal of any type of embedding medium from a biological sample. For example, paraffin-embedded tissue sections are dewaxed by passage through organic solvents, such as toluene, xylene, limonene, or other suitable solvents. In other examples, paraffin-embedded tissue sections are utilized directly (e.g., without a dewaxing step).

Tissues can be fixed by any suitable process, including perfusion or by submersion in a fixative. Fixatives can be classified as cross-linking agents (such as aldehydes, e.g., formaldehyde, paraformaldehyde, and glutaraldehyde, as well as non-aldehyde cross-linking agents), oxidizing agents (e.g., metallic ions and complexes, such as osmium tetroxide and chromic acid), protein-denaturing agents (e.g., acetic acid, methanol, and ethanol), fixatives of unknown mechanism (e.g., mercuric chloride, acetone, and picric acid), combination reagents (e.g., Carnoy's fixative, methacarn, Bouin's fluid, B5 fixative, Rossman's fluid, and Gendre's fluid), microwaves, and miscellaneous fixatives (e.g., excluded volume fixation and vapor fixation). Additives may also be included in the fixative, such as buffers, detergents, tannic acid, phenol, metal salts (such as zinc chloride, zinc sulfate, and lithium salts), and lanthanum. The most commonly used fixative in preparing tissue or cell samples is formaldehyde, generally in the form of a formalin solution (4% formaldehyde in a buffer solution, referred to as 10% buffered formalin). In one example, the fixative is 10% neutral buffered formalin, and thus in some examples the sample is formalin fixed.

In some examples, the sample is an environmental sample (such as a soil, air, air filter, or water sample, or a sample obtained from a surface (for example by swabbing)), or a food sample (such as a vegetable, fruit, dairy or meat containing sample) for example to detect pathogens that may be present.

### VI. Target Nucleic Acids

A target nucleic acid molecule (such as a target DNA or target RNA) is a nucleic acid molecule whose detection, amount, and/or sequence is intended to be determined (for example in a quantitative or qualitative manner), with the disclosed methods. In some examples, DNA is detected directly by amplification of DNA from the sample, while RNA is detected indirectly by the use of a surrogate, such an NPPF. In one example, the target is a defined region or particular portion of a nucleic acid molecule, for example a DNA or RNA of interest. In an example where the target nucleic acid sequence is target DNA and target RNA, such a target can be defined by its specific sequence or function; by its gene or protein name; or by any other means that uniquely identifies it from among other nucleic acids.

In some examples, alterations of a target nucleic acid sequence (e.g., a DNA and/or RNA) are “associated with” a disease or condition. That is, sequencing of the target nucleic acid sequence (either directly or indirectly, such as by detecting or sequencing a surrogate, such as DNA amplicons or NPPF amplicons) can be used to infer the status of a sample with respect to the disease or condition. For example, the target nucleic acid sequence(s) can exist in two (or more) distinguishable forms, such that a first form correlates with absence of a disease or condition and a second (or different) form correlates with the presence of the disease or condition. The two different forms can be qualitatively distinguishable, such as by nucleotide (or ribonucleotide) polymorphisms or mutation, and/or the two different forms can be quantitatively distinguishable, such as by the number of copies of the target nucleic acid sequence that are present in a sample.

Targets include single-, double- and/or other multiple-stranded nucleic acid molecules (such as, DNA (e.g., genomic, mitochondrial, or synthetic), RNA (such as mRNA, miRNA, tRNA, siRNA, long non-coding (nc) RNA, biologically occurring anti-sense RNA, Piwi-interacting RNAs (piRNAs), and/or small nucleolar RNAs (snoRNAs)), whether from eukaryotes, prokaryotes, viruses, fungi, bacteria, parasites, or other biological organisms. Genomic DNA targets may include one or several parts of the genome, such as coding regions (e.g., genes or exons), non-coding regions (whether having known or unknown biological function, e.g., enhancers, promoters, regulatory regions, telomeres, or “nonsense” DNA). In some embodiments, a target may contain or be the result of a mutation (e.g., germ line or somatic mutation) that may be naturally occurring or otherwise induced (e.g., chemically or radiation-induced mutation). Such mutations may include (or result from) genomic rearrangements (such as translocations, insertions, deletions, or inversions), single nucleotide variations, and/or genomic amplifications. In some embodiments, a target may contain one or more modified or synthetic monomer units (e.g., peptide nucleic acid (PNA), locked nucleic acid (LNA), methylated nucleic acid, post-translationally modified amino acid, cross-linked nucleic acid or cross-linked amino acid).

The portion of a target nucleic acid molecule to which a NPPF may specifically bind, or which an amplification primer amplifies, also may be referred to as a “target,” again, as context dictates, but more specifically may be referred to as target portion, complementary region (CR), target site, protected target region or protected site, or similar. A NPPF specifically bound to its complementary region forms a complex, which complex may remain integrated with the target as a whole and/or the sample, or be separate (or be or become separated) from the target as a whole and/or the sample. In some embodiments, a NPPF/CR complex is separated (or becomes disassociated) from the target RNA as a whole and/or the sample.

All types of target nucleic acid molecules can be analyzed using the disclosed methods, such as at least one DNA and at least one RNA. In one example, the target includes a ribonucleic acid (RNA) molecule, such as a messenger RNA (mRNA), a ribosomal RNA (rRNA), a transfer RNA (tRNA), micro RNA (miRNA), an siRNA, anti-sense RNA, or a viral RNA (vRNA). In another example, the target includes a deoxyribonucleic (DNA) molecule, such as genomic DNA (gDNA), mitochondrial DNA (mtDNA), chloroplast DNA (cpDNA), viral DNA (vDNA), cDNA, or a transfected DNA. In a specific example, the target includes an antisense nucleotide. In some examples, the whole transcriptome of a cell or a tissue can be sequenced using the disclosed methods. In one example, one target nucleic acid molecule to be sequenced is a rare nucleic acid molecule, for example only appearing less than about 100,000 times, less than about 10,000 times, less than about 5,000 times, less than about 100 times, less than 10 times, or only once in the sample, such as a nucleic acid molecule only appearing 1 to 10,000, 1 to 5,000, 1 to 100 or 1 to 10 times in the sample.

A plurality of DNA and RNA targets can be sequenced in the same sample or assay, or even in multiple samples or assays, for example simultaneously or contemporaneously. Similarly, a single RNA target and a single DNA can be sequenced in a plurality of samples, for example simultaneously or contemporaneously. In one example the target nucleic acid molecules are a DNA and an RNA (e.g., an miRNA or an mRNA). Thus, in such an example, the method would include the use of at least one set of amplification primers specific for the target DNA, and one NPPF specific for the RNA (e.g., at least one NPPF specific for an miRNA or at least one NPPF specific for an mRNA). In one example, the target nucleic acid molecules include two different RNA molecules. Thus, in such an example, the method could include the use of at least one NPPF specific for the first target RNA and at least one NPPF specific for the second target RNA. In some examples, the DNA target is amplified directly in a least one portion of the sample (e.g., a first portion, such as using at least one target DNA primer), generating FARs. In such examples, the at least one primer (e.g., at least two target DNA primers) can include an extension (e.g., a 5′ and/or 3′ flanking sequence), such as for use in a later amplification step.

In some examples, the disclosed methods permit sequencing of DNA or RNA single nucleotide polymorphisms (SNPs) or variants (sNPVs), splice junctions, methylated DNA, gene fusions or other mutations, protein-bound DNA or RNA, and also cDNA, as well as levels of expression (such as DNA copy number or RNA expression, such as cDNA expression, mRNA expression, miRNA expression, rRNA expression, siRNA expression, or tRNA expression). Any nucleic acid molecule that can be amplified directly and/or to which a NPPF can be designed to hybridize can be quantified and identified by the disclosed methods.

In one example, DNA methylation is detected by using an NPPF that includes a base mismatch at the site where methylation has or has not occurred, such that upon treatment of the target sample, methylated bases are converted to a different base, complementary to the base in the NPPF. Thus, in some examples, the methods include treating the sample with bisulfite.

One skilled in the art will appreciate that the target can include natural or unnatural bases, or combinations thereof.

In specific non-limiting examples, a target nucleic acid (such as a target DNA or target RNA) associated with a neoplasm (for example, a cancer) is selected. Numerous chromosome abnormalities (including translocations and other rearrangements, duplication or deletion) or mutations have been identified in neoplastic cells, especially in cancer cells, such as B cell and T cell leukemias, lymphomas, breast cancer, colon cancer, neurological cancers, and the like.

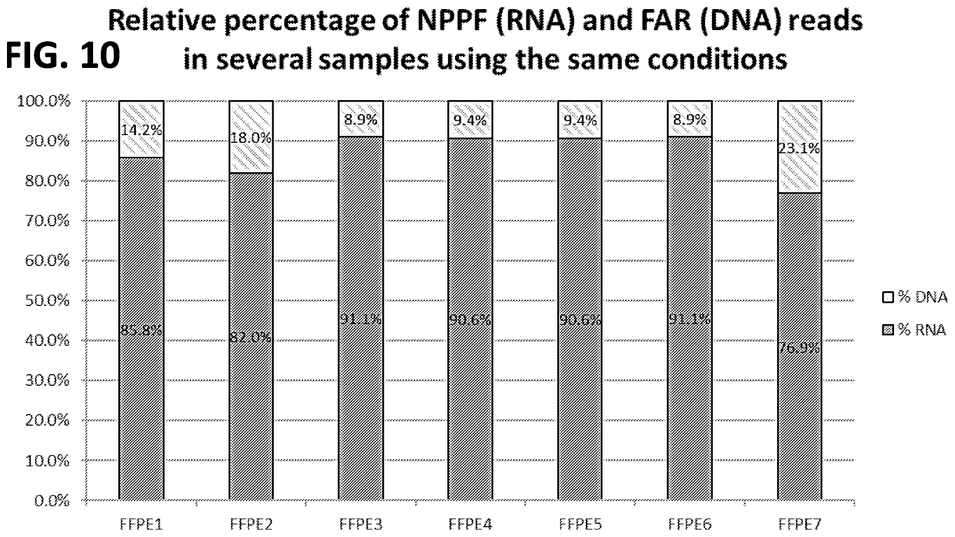

In some examples, a target nucleic acid molecule includes wild type and/or mutated: delta-aminolevulinate synthase 1 (ALAS1) (e.g., GenBank Accession No. NM_000688.5 or OMIM 125290), 60S ribosomal protein L38 (RPL38) (e.g., GenBank Accession No. NM_000999.3 or OMIM 604182), proto-oncogene B-Raf (BRAF) (e.g., GenBank Accession No. NM_004333.4 or OMIM 164757) (such as the wild type BRAF or the V600E, V600K, V600R, V600E2, and/or V600D mutation, e.g., see FIG. 10), forkhead box protein L2 (FOXL2) (e.g., GenBank Accession No. NM_023067.3 or OMIM 605597) (such as the wild type FOXL2 or the nt820 snp C->G); epidermal growth factor receptor (EGFR) (e.g., GenBank Accession No. NM_005228.3 or OMIM 131550) (such as the wild type EGFR, and/or one or more of a T790M, L858R, D761Y, G719A, G719S, and a G719C mutation, or other mutation shown in FIG. 9); GNAS (e.g., GenBank Accession No. NM_000516.5 or OMIM 139320); or KRAS (e.g., GenBank Accession No. NM_004985.4 or OMIM 190070) (such as the wild type KRAS, a D761Y mutation, a G12 mutation such as one or more of G12D, G12V, G12A, G12C, G12S, G12R, a G13 mutation such as G13D and/or a Q61 mutation such as one or more of Q61E, Q61R, Q61L, Q61H-C, and/or Q61H-T).

In some examples, a target nucleic acid molecule includes GAPDH (e.g., GenBank Accession No. NM_002046), PPIA (e.g., GenBank Accession No. NM_021130), RPLPO (e.g., GenBank Accession Nos. NM_001002 or NM_053275), RPL19 (e.g., GenBank Accession No. NM_000981), ZEB1 (e.g., GenBank Accession No. NM_030751), Zeb2 (e.g., GenBank Accession Nos. NM_001171653 or NM_014795), CDH1 (e.g., GenBank Accession No. NM_004360), CDH2 (e.g., GenBank Accession No. NM_007664), VIM (e.g., GenBank Accession No. NM_003380), ACTA2 (e.g., GenBank Accession No. NM_001141945 or NM_001613), CTNNB1 (e.g., GenBank Accession No. NM_001904, NM_001098209, or NM_001098210), KRT8 (e.g., GenBank Accession No. NM_002273), SNAI1 (e.g., GenBank Accession No. NM_005985), SNAI2 (e.g., GenBank Accession No. NM_003068), TWIST1 (e.g., GenBank Accession No. NM_000474), CD44 (e.g., GenBank Accession No. NM_000610, NM_001001389, NM_00100390, NM_001202555, NM_001001391, NM_001202556, NM 001001392, NM_001202557), CD24 (e.g., GenBank Accession No. NM_013230), FN1 (e.g., GenBank Accession No. NM_212474, NM_212476, NM 212478, NM 002026, NM_212482, NM_054034), IL6 (e.g., GenBank Accession No. NM_000600), MYC (e.g., GenBank Accession No. NM_002467), VEGFA (e.g., GenBank Accession No. NM 001025366, NM 001171623, NM_003376, NM_001171624, NM_001204384, NM_001204385, NM 001025367, NM 001171625, NM_001025368, NM_001171626, NM_001033756, NM 001171627, NM 001025370, NM_001171628, NM_001171622, NM_001171630), HIF1A (e.g., GenBank Accession No. NM_001530, NM_181054), EPAS1 (e.g., GenBank Accession No. NM_001430), ESR2 (e.g., GenBank Accession No. NM 001040276, NM 001040275, NM_001214902, NM_001437, NM_001214903), PRKCE (e.g., GenBank Accession No. NM_005400), EZH2 (e.g., GenBank Accession No. NM_001203248, NM_152998, NM_001203247, NM_004456, NM_001203249), DAB2IP (e.g., GenBank Accession No. NM_032552, NM 138709), B2M (e.g., GenBank Accession No. NM_004048), and SDHA (e.g., GenBank Accession No. NM_004168).

In some examples, a target miRNA includes hsa-miR-205 (MIR205, e.g., GenBank Accession No. NR_029622), hsa-miR-324 (MIR324, e.g., GenBank Accession No. NR_029896), hsa-miR-301a (MIR301A, e.g., GenBank Accession No. NR_029842), hsa-miR-106b (MIR106B, e.g., GenBank Accession No. NR_029831), hsa-miR-877 (MIR877, e.g., GenBank Accession No. NR_030615), hsa-miR-339 (MIR339, e.g., GenBank Accession No. NR_029898), hsa-miR-10b (MIR10B, e.g., GenBank Accession No. NR_029609), hsa-miR-185 (MIR185, e.g., GenBank Accession No. NR_029706), hsa-miR-27b (MIR27B, e.g., GenBank Accession No. NR_029665), hsa-miR-492 (MIR492, e.g., GenBank Accession No. NR_030171), hsa-miR-146a (MIR146A, e.g., GenBank Accession No. NR_029701), hsa-miR-200a (MIR200A, e.g., GenBank Accession No. NR 029834), hsa-miR-30c (e.g., GenBank Accession No. NR_029833, NR_029598), hsa-miR-29c (MIR29C, e.g., GenBank Accession No. NR_029832), hsa-miR-191 (MIR191, e.g., GenBank Accession No. NR_029690), or hsa-miR-655 (MIR655, e.g., GenBank Accession No. NR_030391).

In one example the target includes a pathogen nucleic acid, such as viral RNA or DNA. Exemplary pathogens include, but are not limited to, viruses, bacteria, fungi, parasites, and protozoa. In one example, the target includes a viral RNA. Viruses include positive-strand RNA viruses and negative-strand RNA viruses. Exemplary positive-strand RNA viruses include, but are not limited to: Picornaviruses (such as Aphthoviridae [for example foot-and-mouth-disease virus (FMDV)]), Cardioviridae; Enteroviridae (such as Coxsackie viruses, Echoviruses, Enteroviruses, and Polioviruses); Rhinoviridae (Rhinoviruses)); Hepataviridae (Hepatitis A viruses); Togaviruses (examples of which include rubella; alphaviruses (such as Western equine encephalitis virus, Eastern equine encephalitis virus, and Venezuelan equine encephalitis virus)); Flaviviruses (examples of which include Dengue virus, West Nile virus, and Japanese encephalitis virus); and Coronaviruses (examples of which include SARS coronaviruses, such as the Urbani strain). Exemplary negative-strand RNA viruses include, but are not limited to: Orthomyxyoviruses (such as the influenza virus), Rhabdoviruses (such as Rabies virus), and Paramyxoviruses (examples of which include measles virus, respiratory syncytial virus, and parainfluenza viruses). In one example the target includes viral DNA from a DNA virus, such as Herpesviruses (such as Varicella-zoster virus, for example the Oka strain; cytomegalovirus; and Herpes simplex virus (HSV) types 1 and 2), Adenoviruses (such as Adenovirus type 1 and Adenovirus type 41), Poxviruses (such as Vaccinia virus), and Parvoviruses (such as Parvovirus B19). In another example, the target is a retroviral nucleic acid, such as one from human immunodeficiency virus type 1 (HIV-1), such as subtype C, HIV-2; equine infectious anemia virus; feline immunodeficiency virus (FIV); feline leukemia viruses (FeLV); simian immunodeficiency virus (SIV); and avian sarcoma virus. In one example, the target nucleic acid includes a bacterial nucleic acid. In one example the bacterial nucleic acid is from a gram-negative bacteria, such as Escherichia coli (K-12 and O157:H7), Shigella dysenteriae, and Vibrio cholerae. In another example the bacterial nucleic acid is from a gram-positive bacteria, such as Bacillus anthracis, Staphylococcus aureus, pneumococcus, gonococcus, or streptococcal meningitis. In one example, the target nucleic acid includes a nucleic acid from protozoa, nemotodes, or fungi. Exemplary protozoa include, but are not limited to, Plasmodium, Leishmania, Acanthamoeba, Giardia, Entamoeba, Cryptosporidium, Isospora, Balantidium, Trichomonas, Trypanosoma, Naegleria, and Toxoplasma. Exemplary fungi include, but are not limited to, Coccidiodes immitis and Blastomyces dermatitidis.

One of skill in the art can identify additional target DNAs or RNAs and/or additional target miRNAs which can be detected utilizing the methods disclosed herein.

### VII. Assay Output

In some embodiments, the disclosed methods include determining the sequence of one or more target nucleic acid molecules in a sample, which can include quantification of sequences detected. In some example, the sequence of a target RNA is determined indirectly using an NPPF surrogate, such as an amplicon generated from a ssNPPF surrogate (which bound to the target RNA in the sample). In some examples, the sequence of a target DNA is determined directly using a FAR generated from target DNA in the sample. The results of the methods can be provided to a user (such as a scientist, clinician or other health care worker, laboratory personnel, or patient) in a perceivable output that provides information about the results of the test. In some examples, the output can be a paper output (for example, a written or printed output), a display on a screen, a graphical output (for example, a graph, chart, or other diagram), or an audible output. In one example, the output is a table or graph including a qualitative or quantitative indicator of presence or amount (such as a normalized amount) of a target DNA and RNA sequenced (or sequence not detected) in the sample. In other examples, the embodiments, the output is the sequence of one or more target DNA and RNA nucleic acid molecules in a sample, such a report indicting the presence of a particular mutation(s) in the target molecules.

The output can provide quantitative information (for example, an amount of a particular target nucleic acid molecule or an amount of a particular target nucleic acid molecule relative to a control sample or value), or can provide qualitative information (for example, a determination of presence or absence of a particular target nucleic acid molecule). In additional examples, the output can provide qualitative information regarding the relative amount of a target nucleic acid molecule in the sample, such as identifying an increase or decrease relative to a control or no change relative to a control.

As discussed herein, the final amplicons, NPPF amplicons and FAR amplicons, can include one or more experiment tags, which can be used, for example, to identify a particular patient, sample, experiment, or target sequence. The use of such tags permits the sequenced target (e.g., NPPF amplicons for a target RNA or FAR amplicons for a target DNA) to be “sorted” or even counted, and, thus, permits analysis of multiple different samples (for example from different patients), multiple different targets (for example at least two different nucleic acid targets), or combinations thereof in a single reaction. In one example, Illumina and Bowtie 2 or other sequence-alignment software can be used for such analysis.

In one example, the NPPF amplicons for a target RNA and FAR amplicons for a target DNA include an experiment tag unique for each different target nucleic acid molecule. The use of such a tag allows one to merely sequence or detect this tag, without sequencing the entire target (e.g., NPPF amplicons and FAR amplicons), to identify the target (e.g., DNA or RNA target present in the sample). In addition, when multiple nucleic acid targets are analyzed, the use of a unique experiment tag for each target simplifies the analysis, as each detected or sequenced experiment tag can be sorted, and if desired counted. This permits for semi-quantification or quantification of the target nucleic acid that was in the sample as the NPPF amplicons and FAR amplicons are in roughly in stoichiometric proportion to the target in the sample. For example if multiple target nucleic acids are detected or sequenced in a sample, the methods permit the generation of a table or graph showing each target sequence and the number of copies detected or sequenced, by simply detecting or sequencing and then sorting the experimental tag.

In another example, the NPPF amplicons and FAR amplicons include an experiment tag unique for each different sample (such as a unique tag for each patient sample). The use of such a tag allows one to associate a particular detected target (e.g., via NPPF amplicons and FAR amplicons) with a particular sample. Thus, if multiple samples are analyzed in the same reaction (such as the same well or same sequencing reaction), the use of a unique experiment tag for each sample simplifies the analysis, as each detected or sequenced NPPF amplicon and FAR amplicon can be associated with a particular sample. For example if a target nucleic acid is detected or sequenced in samples, the methods permit the generation of a table or graph showing the result of the analysis for each sample.

One skilled in the art will appreciate that each target (e.g., NPPF amplicons and FAR amplicons) can include a plurality of experiment tags (such as at least 2, 3, 4, 5, 6, 7, 8, 9 or 10 experiment tags), such as a tag representing the target sequence and another representing the sample. Once each tag is detected or sequenced, appropriate software can be used to sort the data in any desired format, such as a graph or table. For example, this permits analysis of multiple target sequences in multiple samples simultaneously or contemporaneously. Similarly, the first about 5 to 25 bases of the target region of the NPPF amplicon or FAR amplicon can be sequenced and used to identify the RNA or DNA (i.e., it does not need to be an added tag).

In some examples, the sequenced target (e.g., NPPF amplicons for a target RNA or FAR amplicons for a target DNA) is compared to a database of known sequences for each target nucleic acid sequence. In some examples, such a comparison permits detection of mutations, such as SNVs. In some examples, such a comparison permits for a comparison of a reference NPPF's abundance to the abundance of an NPPF probe, which can represent expression of the target RNA in the sample.

### Example 1

### Simultaneous Sequencing of a Plurality of NPPFs and FARs to Simultaneously Measure RNA Abundance and DNA with Single Base Resolution

This example describes methods used to generate and co-sequence nuclease protection probes with a flanking sequence (NPPFs) and flanked amplicon regions (FARs). A set of 470 NPPFs were designed to RNA targets. Each NPPF was 100 bases in length, included a 50-base region specific for a particular target nucleic acid molecule, and flanking sequences on both the 5′- and 3′-end. The average Tₘ of the 100-base NPPFs was 81.0° C. for all 470 probes (73.2° C. for the protection regions only). A set of four DNA primers were also generated. Each primer set amplified a region of genomic DNA between 50 and 80 bases in size. Each of the four regions encompassed a site known to sometimes have a mutation or mutations. Each primer carried a flanking or extension sequence at its 5′ end. The average Tₘ of the four DNA primer sets was 69.7° C.

In this example, for all NPPFs, regardless of their target, the 5′- and 3′-flanking sequences (FS) differed from one another, but each 5′ FS and each 3′ FS was the same on each NPPF. The 5′-flanking sequence (5′ AGTTCAGACGTGTGCTCTTCCGATC 3′; SEQ ID NO: 1) was 25 nucleotides with a Tₘ of 56° C., and the 3′-flanking sequence (5′ GATCGTCGGACTGTAGAACTCTGAA 3′; SEQ ID NO: 2) was 25 nucleotides with a Tₘ of 53.3° C. In this example, each DNA primer also carried a flanking sequence at the 5′ end. Primers designated as 5′-specific or “forward” primers carried the reverse-complement of the 3′ FS (5′ TTCAGAGTTCTACAGTCCGACGATC 3′; SEQ ID NO: 3), and those primers designated as 3′-specific or “reverse” primers carried the 5′-FS (5′ AGTTCAGACGTGTGCTCTTCCGATC 3′; SEQ ID NO: 1). The full sequences of the four primer sets used are shown in Table 1.

TABLE 1
Primer sets

| Primer name | Sequence (5′ -> 3′) |
|---|---|
| BRAF_V600-F | TTCAGAGTTCTACAGTCCGA |
| | CGATCCAGTAAAAATAGGTG |
| | ATTTTGGTCTAGC |
| | (SEQ ID NO: 4) |
| BRAFV_600-R | AGTTCAGACGTGTGCTCTT |
| | CCGATCCTGATGGGACCCA |
| | CTCCATC |
| | (SEQ ID NO: 5) |
| KRAS_G12F | TTCAGAGTTCTACAGTCCG |
| | ACGATCAAATGACTGAATA |
| | TAAACTTGTGGTAG |
| | (SEQ ID NO: 6) |
| KRAS_G12-R | AGTTCAGACGTGTGCTCTT |
| | CCGATCAATGATTCTGAAT |
| | TAGCTGTAT |
| | (SEQ ID NO: 7) |
| EGFR_T790-F | TTCAGAGTTCTACAGTCCG |
| | ACGATCATCTGCCTCACCT |
| | CCACCG |
| | (SEQ ID NO: 8) |
| EGFR_T790-R | AGTTCAGACGTGTGCTCTT |
| | CCGATCGCAGCCGAAGGGC |
| | ATGA (SEQ ID NO: 9) |
| EGFR_G719-F | TTCAGAGTTCTACAGTCCG |
| | ACGATCTCTTGAGGATCTT |
| | GAAGGAAAC |
| | TGA (SEQ ID NO: 10) |
| EGFR_G719-R | AGTTCAGACGTGTGCTCTT |
| | CCGATCTATACACCGTGCC |
| | GAACGCA |
| | (SEQ ID NO: 11) |

In this example, both formalin-fixed, paraffin-embedded (FFPE) specimens and cell line samples were used. Samples were prepared by addition of sample to a lysis buffer. No extraction of nucleic acids was performed, nor was RNA separated from DNA at any time. To demonstrate the ability to measure DNA mutations, two commercially available cell lines with a known mutation status at their KRAS and BRAF genomic loci were used. The first, LS 174T (“KRAS mut cell line”), is heterozygous for the KRAS 35G>T base change (G12D amino acid change) and is wildtype for BRAF. The second, COLO-205 (“BRAF mut cell line”), carries a BRAF 1799T>A base change (V600E amino acid change) and is wildtype for KRAS. This latter cell line is known to be triploid for the BRAF locus with two of the three loci carrying the mutant allele.

Some of the samples lysed and used in this example were a set of cell line mixtures derived from the two cell lines described above. Cells were diluted in a lysis buffer. Each mixture contained a total of ˜400 cells per microliter of lysis buffer. The two cell lines were mixed together in a ratio dilution series, in which the total number of cells was the same for each sample, but the composition of each sample differed. Eight different samples were formed, as described in Table 2.

TABLE 2
Samples analyzed

| | KRAS mut cell line | BRAF mut cell line |
|---|---|---|
| Sample # | (composition) | (composition) |
| 1 | 100% | 0% |
| 2 | 99% | 10% |
| 3 | 95% | 5% |
| 4 | 90% | 10% |
| 5 | 10% | 90% |
| 6 | 5% | 95% |
| 7 | 1% | 99% |
| 8 | 0% | 100% |

Two portions of lysate from a given sample were used in two separate reactions. One was a nuclease protection reaction to measure the abundance of RNA molecules targeted by the 470 NPPFs described above. The second was an amplification reaction to amplify genomic DNA regions from the sample using the four DNA primer sets described above. In all cases, triplicate reactions were generated. In some cases, triplicate reactions were performed on separate days, for a total of nine replicates per sample.

To measure RNA abundance, the first reaction was constructed with a portion of the lysed material. The 470 NPPFs described above were pooled and hybridized to the sample in solution as well as to CFSs, which are complementary to each of the NPPFs. Hybridization was performed at 50° C. after an initial denaturation at 95° C. Following hybridization, S1 digestion was performed on the hybridized mixture by the addition of S1 enzyme in a buffer. The S1 reaction was incubated at 50° C. for 90 minutes. Following S1-mediated digestion of unhybridized target RNA, NPPFs, and CFSs, the reaction was stopped by addition of the mixture to a fresh vessel containing stop solution. The reaction was heated to 100° C. for 10 minutes and then allowed to cool to room temperature.

In parallel, a second portion of the lysed sample was incubated with a mixture of the four DNA primer sets described above. Ten cycles of amplification were performed using a DNA polymerase that included a proofreading domain.

A portion of the finished nuclease protection experiment (containing NPPFs specific for the target RNAs) and a portion of the finished DNA amplification reaction (containing FARs specific for the target DNAs) were then combined and incubated with DNA primers in a co-amplification reaction. One primer included a sequence that was complementary to the 5′-flanking sequence, and a second primer included a sequence that was complementary to the 3′-flanking sequence. Both primers also included a sequence to allow for incorporation of an experiment tag into the resulting amplicon so that each amplified NPPF or FAR in a single sample amplified using these primers had the same two nucleotide experiment tags. Both primers also included a sequence to allow them to be sequenced using a next-generation sequencing instrument (referred to herein as a sequencing adaptor). Nineteen cycles of amplification were performed.

The first primer, (5′ AATGATACGGCGACCACCGAGATCTACACxxxxxxCGACAGGTTCAGAGTTCTACAGTCC GACG 3′; SEQ ID NO: 12) was 64 bases in length and carried a six-nucleotide experimental tag (designated “xxxxxx” in the sequence above). Twenty-two nucleotides of the primer were exactly complementary to the 3′-flanking region and had a Tₘ of about 50° C.

The second primer: (5′ CAAGCAGAAGACGGCATACGAGATxxxxxxGTGACTGGAGTTCAGACGTGTGCTCTTCC G 3′; SEQ ID NO: 13) was 60 bases in length and carried a six-nucleotide experimental tag (designated “xxxxxx” in the sequence above). Twenty-two of these bases were identical to the 5′-flanking sequence, and had a Tₘ of about 53° C.

The experimental tags designated as “xxxxxx” above were one of the sequences shown in Table 3.

TABLE 3
Exemplary experimental tag sequences.

| | 5′ Barcode | 3′ Barcode |
|---|---|---|
| | sequence | sequence |
| | (5′-3′ in | (5′-3′ in |
| Designation | primer) | primer) |
| F1 | ATTGGC | |
| F2 | GCCAAT | |
| F3 | TGACCA | |
| F4 | CACTGT | |
| F5 | TAGCTT | |
| F6 | ACATCG | |
| F7 | TGGTCA | |
| F8 | CTGATC | |
| F9 | GATCTG | |
| R1 | | ACATCG |
| R2 | | ATTGGC |
| R3 | | GATCTG |
| R4 | | TTAGGC |
| R5 | | ACAGTG |
| R6 | | GCCAAT |
| R7 | | ACTTGA |
| R8 | | CGTACG |

Each reaction was amplified in a separate PCR reaction, and each was amplified with a different combination of experimental tags, so each reaction could be separately identified following sequencing of the pooled reactions.

The samples (containing both NPPF amplicons and FAR amplicons, “tagged” with their unique experimental tags) were then individually cleaned up using a bead-based sample cleanup (AMPure XP PCR™ from BeckmanCoulter). Each sample was individually quantified, and an equal amount of each sample was combined together into one library pool for sequencing. Sequencing was performed on an Illumina sequencer. While the experimental tags can be located in several places, in this example, they were located at both sides of the amplicon, immediately adjacent to a region complimentary to an index-read sequencing primer. Thus, Illumina sequencing was performed in three steps, which included an initial read of the sequence followed by two shorter reads of the experimental tags using two other sequencing primers. The sequencing method described herein and used is a standard method for sequencing multiplexed samples on an Illumina platform.

Following sequencing, each molecule sequenced was first sorted by sample based on the experimental tags; next, within each experiment tag group, the number of molecules identified for each of the different tags was counted. Sequence results, whether stemming from NPPFs or FARs, were compared to the expected sequences using the open-source software Bowtie 2 (Langmead B and Salzberg S., Nature Methods., 2012, 9:357-359).

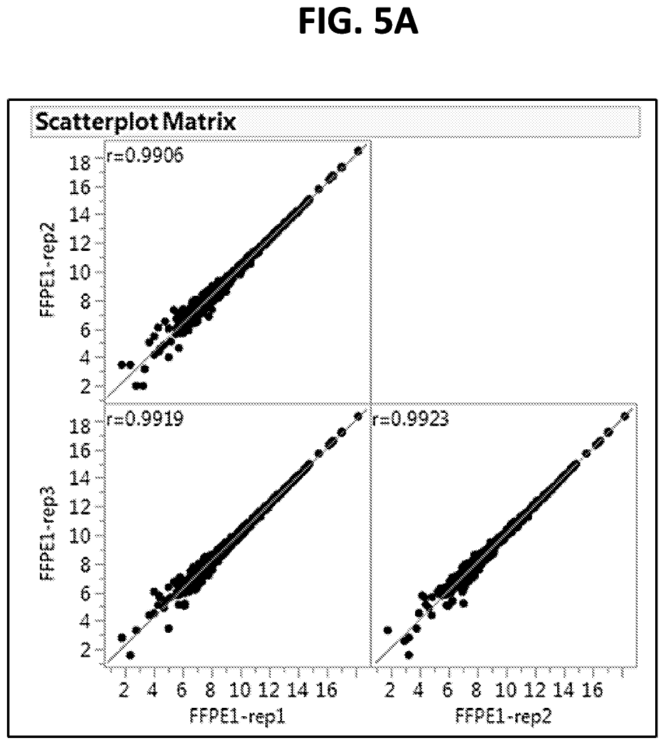

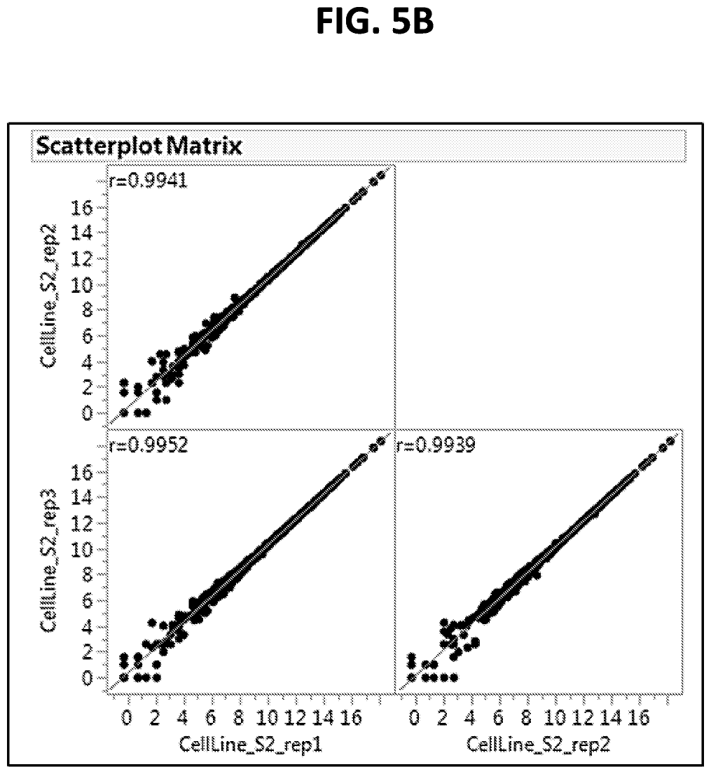

FIGS. 5-8 show the results from sequencing the combined reactions. First, the measurement of RNA expression was highly repeatable, as demonstrated by Pearson correlations of greater than 0.95 for triplicate samples (FIGS. 5A-5B). The data shown are raw data for the 470 RNA measurements, log₂-transformed. Pairwise correlations have been plotted for each comparison shown with the r value for the comparison clearly shown in the graph. Triplicate results for two samples are shown as examples: one FFPE sample (FIG. 5A) and one cell line mixture sample (FIG. 5B).

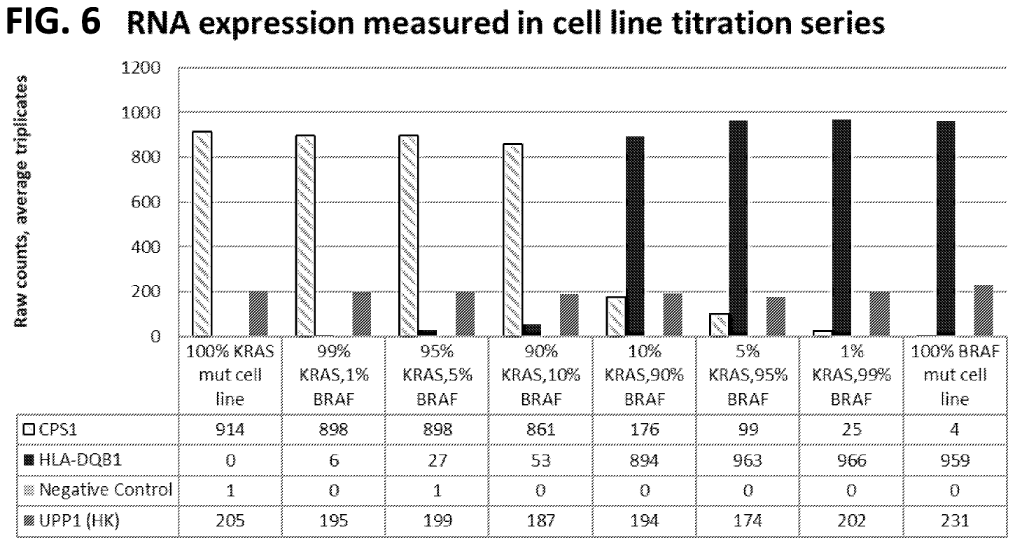

Second, RNA expression was measured throughout the titration series. The expression data from four elements (HLA-DQB1, CPS1, UPP1, and the assay negative control) were plotted across the titration series and are consistent with known expression in the cell lines. For each sample, the average of triplicate experiments was used. The raw data from the triplicate experiments were standardized (the total number of counts for each sample was set as equal, and each signal was re-calculated as a proportion of the total counts). The graph in FIG. 6 shows the results for the four elements. CPS1 is well-expressed in the 100% KRAS cell line sample, but is not expressed in the 100% BRAF sample, while HLA-DQB1 shows the opposite pattern. The expression level of these two transcripts changes across the titration series based on the 100% cell line results. For the two control elements, UPP1 was used as a “housekeeper” (labeled “HK” on the graph) because it does not change between the cell lines and, thus, remains constant across the titration series. The assay negative control is also shown, which is and should be zero or close to zero for all samples.

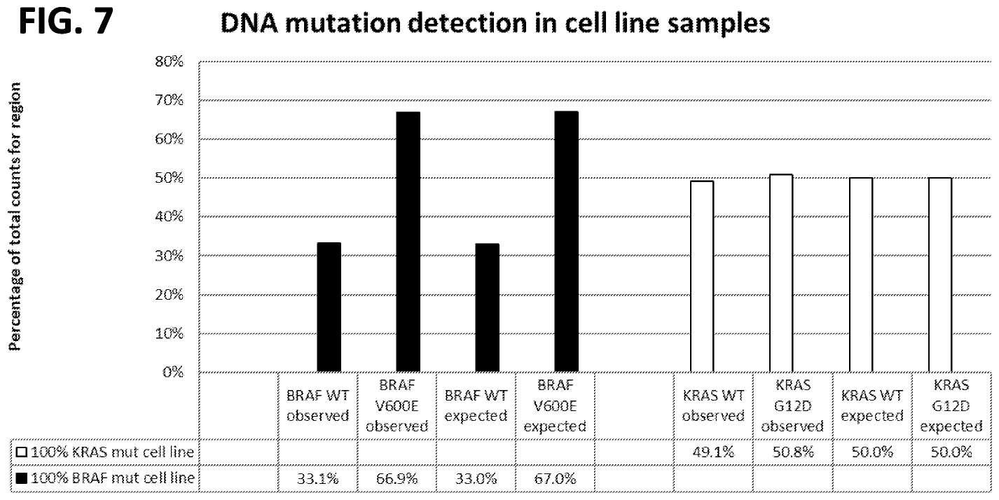

Third, the data show that DNA mutations can be measured at single-base resolution and reliably generate results that are consistent with known mutations in these cell lines. This is exemplified in the DNA results for the 100% samples with each cell line (Samples 1 and 8 in Table 1) in FIG. 7. In the BRAF cell line, a ˜67% composition of BRAF V600E and a ˜33% of wildtype BRAF is expected (remember that this cell line is known to be triploid at the BRAF locus, so three copies are expected, two of which carry the V600E mutation). In the KRAS cell line, which is heterozygous for the G12D mutation, a 50%-50% ratio of WT and mutation is expected. The data in FIG. 7 were generated by averaging the raw counts for nine replicates (triplicates measurements on three different days) of Samples 1 and 8. The total counts measuring the entire locus (BRAF or KRAS) was set to 100%, and the counts for the mutant or WT sequences were calculated as a percentage of those total counts. The data are labeled “observed” and are graphed next to the expected values (labeled “expected”). It is clear from these results that the DNA mutation status of the cell lines is correctly measured using the methods described above.

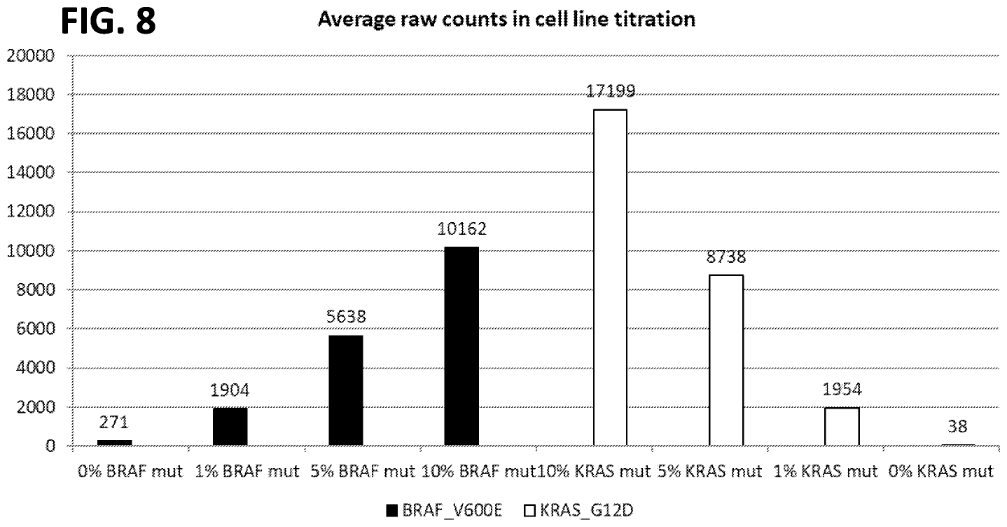

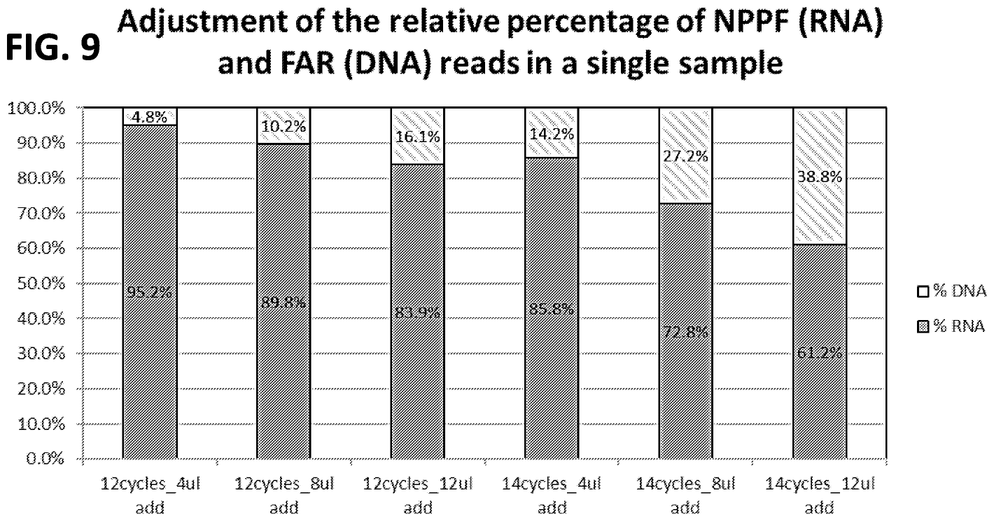

Finally, a mutated allele can be reliably measured using these methods even when the mutation is present at less than 1% of the total sequences for that locus. This is demonstrated by the titration series of the two cell lines (Samples 1-8). In Samples 2 and 7, one mutation-carrying cell lines is present at only 1% of the total sample (˜10 cells). In both cases, the mutation carried by the 1% cell line is clearly and reliably measured, and the measurement is well above the background. The data in FIG. 8 were generated by averaging the raw values for nine measurements per sample (triplicates run on three different days) and graphing the raw values. Notably, each mutation is present in heterozygous form within the cell lines. Thus, the methods can discern single-base changes in genomic DNA even when less than 1% of the measured locus carries a mutation within the sample.

These results demonstrate that the disclosed methods can both reliably measure the expression levels of multiple RNAs and discern single-base changes of multiple genomic regions, even when the single-base changes are present at less than 1% of the total DNA at the given locus.

### Example 2

### Adjusting Relative Amounts of NPPFs and FARs

This example describes methods used to generate and sequence NPPFs and FARs as well as to adjust the balance of NPPF (RNA) and FAR (DNA) reads in the final sample. This example demonstrates that the balance can be adjusted using one or more described parameters. This adjustment aids in assay flexibility; and the desired total signal and signal balance can be modeled prior to experimentation.

The four DNA primer sets and 470 NPPFs used were as described in Example 1. A set of seven samples was generated. These samples were commercially procured, formalin-fixed, paraffin-embedded (FFPE), 5 microns thick, and mounted on glass slides. The samples were lysed by addition of the sample to a lysis buffer at 0.5 mm² of tissue per microliter of lysis buffer. No RNA or DNA extraction was performed. Portions of the lysed samples were used in two reactions. As in Example 1, one was a nuclease protection reaction to measure the abundance of RNA molecules targeted by the 470 NPPFs. The second was an amplification reaction to amplify genomic DNA regions from the sample, using the four DNA primer sets.

To measure RNA abundance, the first reaction was set up with a portion of the lysed material. The 470 NPPFs described above were pooled and hybridized to the sample in solution as well as to CFSs that are exactly complementary to the FS on the NPPFs. Hybridization was performed at 50° C. after an initial denaturation at 85° C. Following hybridization, S1 digestion was performed on the hybridized mixture by the addition of S1 enzyme in a buffer. The S1 reaction was incubated at 50° C. for 90 minutes. Following S1-mediated digestion of the unhybridized target RNA, NPPFs, and CFSs, the reaction was stopped by addition of the mixture to a fresh vessel containing stop solution. The reaction was heated to 100° C. for 10 minutes and then allowed to cool to room temperature.

In parallel, a second portion of the lysed sample was incubated with a mixture of the four DNA primer sets described in Example 1. Twelve or 14 cycles of amplification were performed using a DNA polymerase. Each sample was amplified once at each cycle number.

A portion of the finished nuclease protection experiment (NPPFs) and a portion of the finished DNA amplification reaction (FARs) were then combined and incubated with DNA primers in a co-amplification reaction. In all cases, a constant four microliters of the NPPFs reaction was used, but the 12-cycle or 14-cycle FARs were added at either 4 microliters (1-to-1), 8 microliters (2-to-1), or 12 microliters (3-to-1), for a total of 6 different co-amplification reactions per sample. The DNA primers used in the co-amplification reaction are exactly as described in Example 1; they included a sequence to allow for incorporation of an experiment tag into the resulting amplicon and a sequence to allow them to be sequenced using a next-generation sequencing instrument. Nineteen cycles of amplification were performed. Each reaction was amplified in a separate PCR reaction, and each was amplified with a different combination of experimental tags, so each reaction could be separately identified following sequencing of the pooled reactions

For one of the seven samples, the reaction conditions (DNA amplification cycles and amount of FARs added to the co-amplification reaction) and the sequences of the experimental tags for the co-amplification reaction are displayed in Table 4. The other six samples were treated identically, except that the experimental tag combination assigned to each sample and condition were unique.

TABLE 4
Reaction conditions

| | Amplifi- | FARs | | 5′ | | 3′ |
|---|---|---|---|---|---|---|
| | cation | added | | Barcode | | Barcode |
| | cycles, | to | 5′ | sequence | | sequence |
| | DNA | second | prim- | (5′-3′ | 3′ | (5′-3′ |
| Sample | amplifi- | PCR | er | in | primer | in |
| Name | cation | (μl) | name | primer) | name | primer) |
| FFPE1_ | 12 | 4 | F1 | ATTGGC | R1 | AAGCTA |
| 124 | | | | | | |
| FFPE1_ | 14 | 4 | F2 | GCCAAT | R1 | AAGCTA |
| 144 | | | | | | |
| FFPE1_ | 12 | 8 | F3 | TGACCA | R1 | AAGCTA |
| 128 | | | | | | |
| FFPE1_ | 14 | 8 | F4 | CACTGT | R1 | AAGCTA |
| 148 | | | | | | |
| FFPE1_ | 12 | 12 | F5 | TAGCTT | R1 | AAGCTA |
| 1212 | | | | | | |
| FFPE1_ | 14 | 12 | F6 | ACATCG | R1 | AAGCTA |
| 1412 | | | | | | |

The samples (containing both NPPF amplicons and FAR amplicons, now “tagged” with their unique experimental tags) were then individually cleaned up using bead-based sample cleanup (AMPure XP from BeckmanCoulter). Each sample was individually quantified, and an equal amount of each sample was combined together into one library pool for sequencing. Sequencing was performed on an Illumina sequencer. While the experimental tags can be located in several places, in this example, they were located at both sides of the amplicon, immediately adjacent to a region complimentary to an index-read sequencing primer. Thus, Illumina sequencing was performed in three steps, including an initial read of the sequence followed by two shorter reads of the experimental tags using two other sequencing primers. The sequencing method described herein and used is a standard method for sequencing multiplexed samples on an Illumina platform. Following sequencing, each molecule sequenced was first sorted by sample based on the experimental tags; next, within each experiment tag group, the number of molecules identified for each of the different tags was counted. Sequence results, whether stemming from NPPFs or FARs, were compared to the expected sequences using the open-source software Bowtie 2 (Langmead and Salzberg, Nature Methods., 2012, 9:357-359.).

FIGS. 9-10 show the results from sequencing the combined reactions for a single sample and show that the described methods can be used to adjust the balance between NPPF (RNA) and FAR (DNA) signals assigned to an individual sample. The graph displayed in FIG. 9 shows the percentage of total reads consumed by NPPFs/RNA (grey) and by FARs/DNA (hatched grey) for one sample under the different conditions used. In this sample, the DNA reads resulting from adjustment of the amplification cycles and addition to the co-amplification reaction ranges from about 5% to about 40%. Thus, this range can be altered still further by adjusting either amplification cycle number or material added to the co-amplification reaction. Thus, both amplification cycles in the initial DNA amplification and the volume of FARs added to the co-amplification reaction are adjustable conditions. A third adjustable parameter is the detector (in this example, a sequencer with a particular kit and a particular innate error rate) used for measurement.

The results also demonstrate that the relative percentages of DNA and RNA analytes measured remains constant among different samples using the disclosed methods. FIG. 10 shows the results for a single set of conditions (14 cycles and 4 ul added) for all seven FFPE samples. As in FIG. 9, the graph shows the percentage of total reads consumed by NPPFs or RNA (grey) and by FARs or DNA (hatched grey). FIG. 10 demonstrates that, for a given set of conditions, the RNA and DNA percentages measured in different samples is similar, albeit within a range.

This example demonstrates that the methods described herein allow the number of DNA regions and/or the number of NPPFs measured to be flexible. The desired total signal and signal assigned to either component can, therefore, change based on the total number of analytes, the relative number of different types of analyte, the desired limit of detection (sensitivity) of the measurements, and the capacity of the detector (in this example, counts, or number of sequencing reads, on the sequencer). The detector influences sensitivity in two ways both via the capacity or number of total signals it will generate and by innate error of the detector system, such as an error in basecalling during sequencing. The parameters described above may all be modeled to give a theoretical number of total reads and relative percentages for a particular set of conditions, which, in turn, provides the acceptance criteria for judging the success of “tuning” or adjustment experiments for an assay(s).

### Example 3

### Simultaneous Assessment of Clinical FFPE Samples for BRAF Mutation and RNA Expression Status

This example describes methods used to assess both RNA expression and BRAF genomic mutation status in a set of eight commercially available, formalin-fixed, paraffin-embedded (FFPE) lung and melanoma samples with a known BRAF genomic mutational status.

For this example, the four DNA primer sets and 470 NPPFs used were as described in Example 1. FFPE samples were cut in 5 micron-thick sections and mounted on glass slides prior to use. Samples were lysed by addition of sample to a lysis buffer at 0.17 mm² of tissue per microliter of lysis buffer. No RNA or DNA extraction was performed. Portions of the lysed samples were used in two reactions, as described in Example 1. One reaction was a nuclease protection reaction to measure the abundance of RNA molecules targeted by the 470 NPPFs. The second was an amplification reaction to amplify genomic DNA regions from the sample using the four DNA primer sets. Each sample was run in triplicate.

To measure RNA abundance, the first reaction was set up with a portion of the lysed material. The 470 NPPFs described above were pooled and hybridized to the sample in solution as well as to CFSs that were complementary to the flanking regions on the NPPFs. Hybridization was performed at 50° C. after an initial denaturation at 85° C. Following hybridization, S1 digestion was performed on the hybridized mixture by the addition of S1 enzyme in a buffer. The S1 reaction was incubated at 50° C. for 90 minutes. Following S1-mediated digestion of the unhybridized target RNA, NPPFs, and CFSs, the reaction was stopped by addition of the mixture to a fresh vessel containing stop solution. The reaction was heated to 100° C. for 10 minutes and then allowed to cool to room temperature.

In parallel, a second portion of the lysed sample was incubated with a mixture of the four DNA primer sets described above. Ten cycles of amplification were performed using a DNA polymerase or mixture of polymerases that included a proofreading domain.

A portion of the finished nuclease protection experiment and a portion of the finished DNA amplification reaction were then combined and incubated with DNA primers in a co-amplification reaction. The primers used in this co-amplification reaction were as described in Examples 1 and 2. Nineteen cycles of amplification were performed. Each reaction was amplified in a separate PCR reaction, and each was amplified with a different combination of experimental tags, so each reaction could be separately identified following sequencing of the pooled reactions. Experimental tags used are shown in Table 5.

TABLE 5
Experimental Tags

| | 5′ | 3′ |
|---|---|---|
| | Barcode | Barcode |
| | sequence | sequence |
| | (5′-3′ | (5′-3′ |
| | in | in |
| Designation | primer) | primer) |
| F1 | ATTGGC | |
| F2 | GCCAAT | |
| F3 | TGACCA | |
| R1 | | ACATCG |
| R2 | | ATTGGC |
| R3 | | GATCTG |
| R4 | | TTAGGC |
| R5 | | ACAGTG |
| R6 | | GCCAAT |
| R7 | | ACTTGA |
| R8 | | CGTACG |

The samples (containing both NPPF amplicons and FAR amplicons, now “tagged” with their unique experimental tags) were then individually cleaned up using bead-based sample cleanup (AMPure XP from BeckmanCoulter). Each sample was individually quantified, and an equal amount of each sample was combined together into one library pool for sequencing. Sequencing was performed on an Illumina sequencer. While the experimental tags can be located in several places, in this example, they were located at both sides of the amplicon, immediately adjacent to a region complimentary to an index-read sequencing primer. Thus, Illumina sequencing was performed in three steps, including an initial read of the sequence followed by two shorter reads of the experimental tags using two other sequencing primers. The sequencing method described herein and used is a standard method for sequencing multiplexed samples on an Illumina platform. Following sequencing, each molecule sequenced was first sorted by sample based on the experimental tags; next, within each experiment tag group, the number of molecules identified for each of the different tags was counted. Sequence results, whether stemming from NPPFs or FARs, were compared to the expected sequences using the open-source software Bowtie 2 (Langmead and Salzberg, Nature Methods., 2012, 9:357-359).

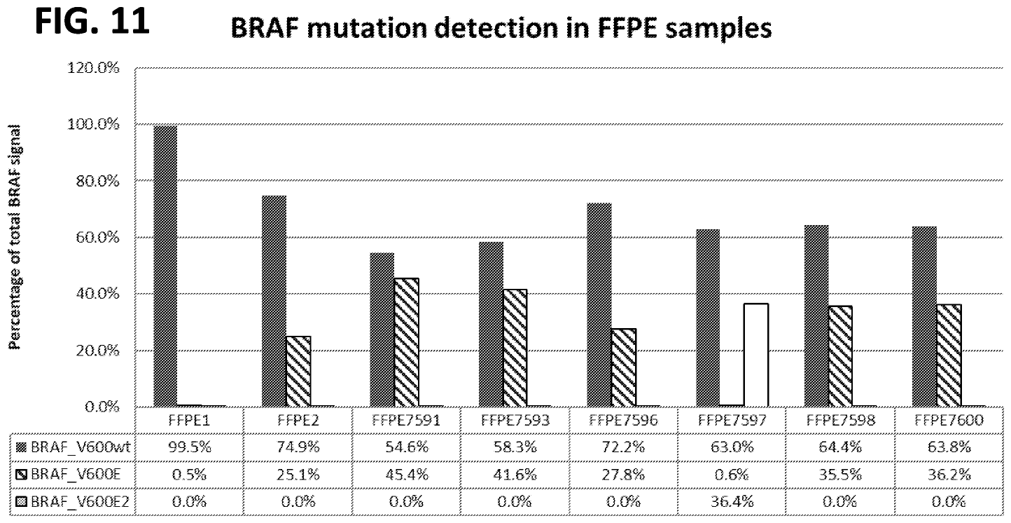

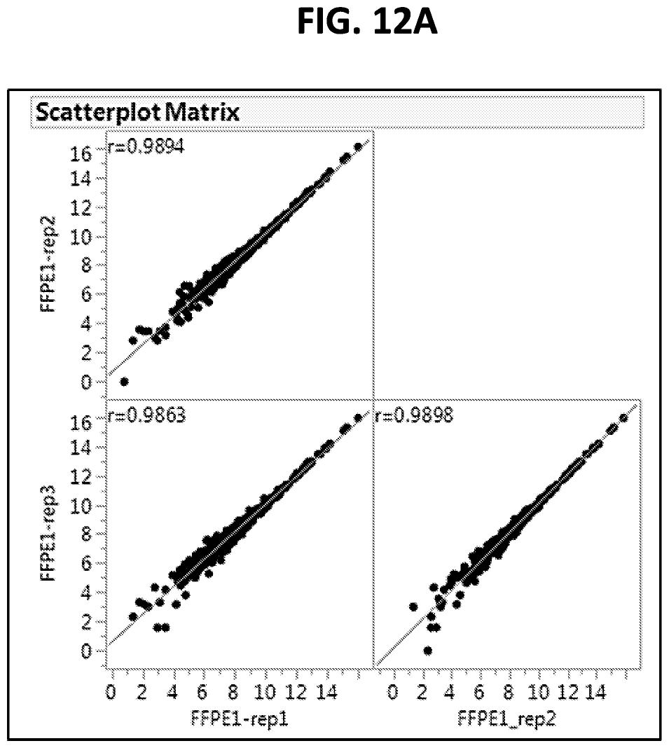

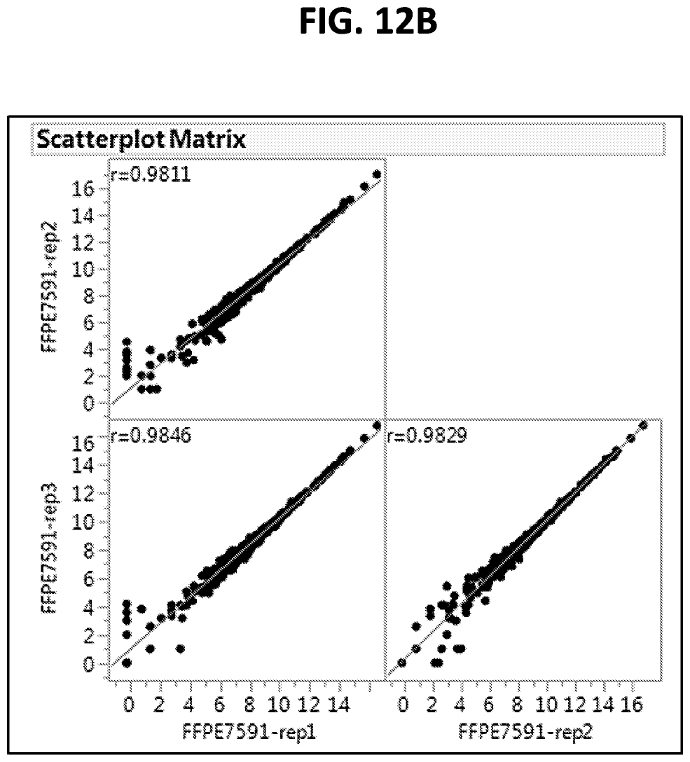

FIGS. 11-12 show the results from co-sequencing the NPPFs and FARs from each sample. DNA mutation information is shown in FIG. 11. The graph displayed was generated by first averaging the raw counts from triplicate samples. The total number of counts generated from the BRAF region, whether wildtype or mutant was summed, and the proportion of wildtype or mutant signal for each sample was calculated. These proportions are shown in the graph in FIG. 11. This figure also displays the BRAF genomic sequence. The wildtype sequence is shown at the top of the figure, with two known mutations (V600E and V600E2) below and the changes marked in red.

These data demonstrate that the described methods can be used to correctly identify the BRAF V600E mutation within these clinical FFPE samples. Three observations were made. One, a single sample carried the V600E2 mutation (sequences shown in the figure), demonstrating the ability of these methods to differentiate between these two similar mutations. The sample was described by the vendor as carrying a “V600E” mutation, but previous sequencing of this sample had shown that the E2 mutation was present. These two mutations have the same effect (amino acid change V>E) and cannot be differentiated by most PCR-based assays, meaning that the vendor was likely unaware of the exact mutation. This result demonstrates that the disclosed methods can uncover unknown mutations s. Third, the results for FFPE1 and FFPE2, both lung cancer samples, closely match their previously-generated exome-sequencing data; the allelic frequency for the V600E mutation in FFPE2 was estimated at 0.22 by exome sequencing and was estimated at 0.25 using the methods described herein. FFPE1 was shown by exome sequencing to be wildtype for BRAF and is clearly also wildtype in this example.

FIGS. 12A-12B and the table below display aspects of the RNA expression data generated for these eight samples. Pearson correlations for triplicate measurements of the entire 470 NPPF set are shown for FFPE1 (lung, FIG. 12A) and FFPE7591 (melanoma, FIG. 12B). All correlations are excellent, with r values greater than 0.95. Data shown are raw data, log₂ transformed. The measured expression level for a few relevant transcripts are shown for two samples, again for FFPE1 (lung adenocarcinoma) and FFPE7591 (melanoma) (see Table 3). The lung cancer specimen is known to be an adenocarcinoma and clearly shows strong expression of lung-specific markers, such as MUC1 and SFTPA2, and adenocarcinoma markers KRT7 and NAPSA. The melanoma sample, conversely, shows strong expression of melanocyte markers PMEL and TYR and melanoma markers SOX10 and MITF. The levels of positive and negative assay control elements and B2M (a housekeeper) are also shown for each sample to demonstrate the similarity of these measurements between samples. The data displayed in Table 6 are an average of raw data for triplicate samples, standardized (see Example 1) to set the total counts for each sample equal to one another.

TABLE 6
RNA Expression levels in lung cancer and melanoma samples

| | FFPE1 | FFPE7951 |
|---|---|---|
| Sample | (lung adenocarcinoma) | (melanoma) |
| Negative control | 1 | 2 |
| Positive control | 1502 | 1172 |
| B2M (housekeeper) | 12887 | 10161 |
| KRT7 | 3820 | 25 |
| NAPSA | 17526 | 206 |
| MUC1 | 4928 | 140 |
| SFTPA2 | 71536 | 161 |
| PMEL | 15 | 95282 |
| TYR | 341 | 26976 |
| MLANA | 290 | 9711 |
| SOX10 | 65 | 10884 |
| MITF | 196 | 4758 |

This example demonstrates the ability of the described methods to co-detect both RNA expression and DNA mutation status within fixed, clinically-relevant samples. DNA mutations are clearly discriminated at the single-base level, such as between the BRAF V600E and V600E2 mutations carried by the samples assessed within this example. Measurement of RNA expression in replicate samples is highly repeatable, and expected markers are expressed by samples of known tissue origin. Additionally, these results were generated using a parsimonious amount (˜6 mm²) of fixed tissue with no RNA or DNA extraction, demonstrating the ability of the described methods to work well using small amounts of clinically-relevant samples.

### Example 4

### Simultaneous Assessment of FFPE Reference Standards for DNA Mutations, Insertions, and Deletions, and RNA Expression Status Using an NPPF and FAR Assay

This example describes methods used to generate and co-sequence NPPFs and FARs in three separate, individual samples, from three types of cancer. In this example, the samples utilized were commercially-available, characterized reference standards, carrying known DNA variations at known allelic frequencies. Data generated for these samples by the disclosed methods, described below, were compared to the expected results reported by the vendor of the reference material.

For this example, the 470 NPPFs used were as described in Example 1.

For this example, a set of eight DNA primer pairs to generate FARs was designed. As in the previous examples, each DNA primer carried a flanking sequence at the 5′ end. Primers designated as 5′-specific or “forward” primers carried the reverse-complement of the 3′ FS (5′ TTCAGAGTTCTACAGTCCGACGATC 3′, SEQ TD NO: 3), and those primers designated as 3′-specific or “reverse” primers carried the 5′-FS (5′ AGTTCAGACGTGTGCTCTTCCGATC 3′ SEQ ID NO: 1). The full sequences of the eight primer sets used are displayed in Table 7. Each of these primers included a phosphorothioate linkage between the last two bases at their 3′ end.

TABLE 7
Primers used

| | Sequence |
|---|---|
| Primer name | (5′->3′) |
| BRAF_V600_F | TTCAG |
| | AGTTC |
| | TACAG |
| | TCCGA |
| | CGATC |
| | TGTTC |
| | AAACT |
| | GATGG |
| | GACC |
| | (SEQ ID |
| | NO: 17) |
| BRAF_V600_R | AGTTC |
| | AGACG |
| | TGTGC |
| | TCTTC |
| | CGATC |
| | CATGA |
| | AGACC |
| | TCACA |
| | GTAAA |
| | (SEQ ID |
| | NO: 18) |
| EGFR_G719 F | TTCAG |
| | AGTTC |
| | TACAG |
| | TCCGA |
| | CGATC |
| | CCAGG |
| | GACCT |
| | TACCT |
| | TATAC |
| | (SEQ ID |
| | NO: 19) |
| EGFR_G719 R | AGTTC |
| | AGACG |
| | TGTGC |
| | TCTTC |
| | CGATC |
| | GCTCT |
| | CTTGA |
| | GGATC |
| | TTGAA |
| | (SEQ ID |
| | NO: 20) |
| EGFR_Ex19-D761_F | TTCAG |
| | AGTTC |
| | TACAG |
| | TCCGA |
| | CGATC |
| | CACAC |
| | AGCAA |
| | AGCAG |
| | AAAC |
| | (SEQ ID |
| | NO: 21) |
| EGFR_Ex19-D761_R | AGTTC |
| | AGACG |
| | TGTGC |
| | TCTTC |
| | CGATC |
| | CCAGA |
| | AGGTG |
| | AGAAA |
| | GTTAA |
| | (SEQ ID |
| | NO: 22) |
| EGFR_Ex20_F | TTCAG |
| | AGTTC |
| | TACAG |
| | TCCGA |
| | CGATC |
| | CAGGA |
| | AGCCT |
| | ACGTG |
| | ATG |
| | (SEQ ID |
| | NO: 23) |
| EGFR_Ex20_R | AGTTC |
| | AGACG |
| | TGTGC |
| | TCTTC |
| | CGATC |
| | AGCCG |
| | AAGGG |
| | CATGA |
| | G |
| | (SEQ ID |
| | NO: 24) |
| EGFR_L858_F | TTCAG |
| | AGTTC |
| | TACAG |
| | TCCGA |
| | CGATC |
| | CACCG |
| | CAGCA |
| | TGTCA |
| | A |
| | (SEQ ID |
| | NO: 25) |
| EGFR_L858-L861_R | AGTTC |
| | AGACG |
| | TGTGC |
| | TCTTC |
| | CGATC |
| | ACCTA |
| | AAGCC |
| | ACCTC |
| | CTT |
| | (SEQ ID |
| | NO: 26) |
| KRAS_G12_F | TTCAG |
| | AGTTC |
| | TACAG |
| | TCCGA |
| | CGATC |
| | ATTCT |
| | GAATT |
| | AGCTG |
| | TATCG |
| | T |
| | (SEQ ID |
| | NO: 27) |
| KRAS_G12_R | AGTTC |
| | AGACG |
| | TGTGC |
| | TCTTC |
| | CGATC |
| | ATGAC |
| | TGAAT |
| | ATAAA |
| | CTTGT |
| | GGT |
| | (SEQ ID |
| | NO: 28) |
| KRAS_Q61_F | TTCAG |
| | AGTTC |
| | TACAG |
| | TCCGA |
| | CGATC |
| | GCAAG |
| | TAGTA |
| | ATTGA |
| | TGGAG |
| | AA |
| | (SEQ ID |
| | NO: 29) |
| KRAS_Q61_R | AGTTC |
| | AGACG |
| | TGTGC |
| | TCTTC |
| | CGATC |
| | GGCAA |
| | ATACA |
| | CAAAG |
| | AAAGC |
| | (SEQ ID |
| | NO: 30) |
| PIK3CA_F | TTCAG |
| | AGTTC |
| | TACAG |
| | TCCGA |
| | CGATC |
| | AAAGC |
| | AATTT |
| | CTACA |
| | CGAGA |
| | T |
| | (SEQ ID |
| | NO: 31) |
| PIK3CA_R | AGTTC |
| | AGACG |
| | TGTGC |
| | TCTTC |
| | CGATC |
| | ACTTA |
| | CCTGT |
| | GACTC |
| | CATAG |
| | (SEQ ID |
| | NO: 32) |

To demonstrate the ability of the described technique to measure DNA mutations, characterized reference standards—with known mutations present at known allelic frequencies—were obtained from Horizon Discovery. Three such reference samples were obtained (HD300, HD301, HD789). These samples were obtained as FFPE sections. Samples were prepared by addition of the FFPE section to a lysis buffer. No extraction of nucleic acids was performed, nor was RNA separated from DNA at any time.

Each lysed sample was run separately and as part of a mixture, for a total of six samples. Mixtures were designed to allow measurement of mutations at allelic frequencies of 100 or less, and were generated by diluting one lysed sample into another at a 20%:80% ratio.

Two portions of lysate from one sample (HD300, HD301, or HD789) were used in two separate reactions. The total input used for each reaction was ˜1000 cells. One portion was used for a nuclease protection reaction to measure the abundance of RNA molecules, targeted by the 470 NPPFs described above. The second portion was used for an amplification reaction to amplify genomic DNA regions from the sample, using the DNA primers set described above. In all cases, triplicate reactions were run. Triplicate reactions were run on separate days, for a total of nine replicates per sample.

To measure RNA abundance, the nuclease protection reaction was set up with a first portion of the lysed material. The 470 NPPFs described above were pooled, and hybridized to the sample in solution, as well as to oligonucleotides called CFSs—these are exactly complementary to the flanking regions on the NPPFs. Hybridization was performed at 50° C. after an initial denaturation at 85° C. Following hybridization, S1 digestion was performed on the hybridized mixture by the addition of S1 enzyme in a buffer. The S1 reaction was incubated at 50° C. for 90 minutes. Following S1-mediated digestion of unhybridized target RNA, NPPFs, and CFSs, the reaction was stopped by addition of the mixture to a fresh vessel containing stop solution. The reaction was heated to 100° C. for 10 minutes and then allowed to cool to room temperature.

In parallel, a second portion of the lysed sample was incubated with a mixture of the DNA primer sets described above. Ten cycles of amplification were performed using a DNA polymerase or mixture of polymerases that included a proofreading domain. The PCR reactions were cleaned up using bead-based sample cleanup (AMPure XP from BeckmanCoulter).

A portion of the finished nuclease protection experiment and a portion of the cleaned-up DNA amplification reaction were then combined and incubated with DNA primers in a co-amplification reaction, as described in the previous examples. Nineteen cycles of amplification were performed.

Each reaction was amplified in a separate PCR reaction, and each was amplified with a different combination of experimental tags, so each reaction could be separately identified following sequencing of the pooled reactions. Samples were pooled by triplicate and the pools cleaned up using bead-based sample cleanup (AMPure XP from BeckmanCoulter). Each pool was individually quantified, and an equal amount of each pool was combined together into one library pool for sequencing. Paired-end sequencing was performed on an Illumina sequencer, with 100 cycles of sequencing on each end and two tag-specific reads of 6 bases each. The experimental tags were located in the library at both sides of the amplicon, immediately adjacent to a region complimentary to an index-read sequencing primer. Illumina sequencing was performed in four steps: An initial read of the sequence followed by two shorter reads of the experimental tags using two other sequencing primers, and finally a second read of the insert, from the opposite end. The sequencing method described herein and used is a standard method for paired-end sequencing of multiplexed samples on an Illumina platform.

Following sequencing, each molecule sequenced was first sorted by sample, or demultiplexed, based on the experimental tags. Demultiplexed fastq files were processed twice to extract DNA and RNA information. For the latter (RNA), fastq files were aligned to expected NPPF sequences using the open-source software Bowtie 2 (Langmead and Salzberg, Fast gapped-read alignment with Bowtie 2. Nature Methods. 2012, 9:357-359), and counts for each alignment compiled. Counts data were log 2 transformed and standardized prior to PCA analysis. For the former (DNA), fastq files were aligned to genomic sequences, also using Bowtie2, the counts for each region and variant compiled, and the total counts for each amplicon region set equal to 100%.

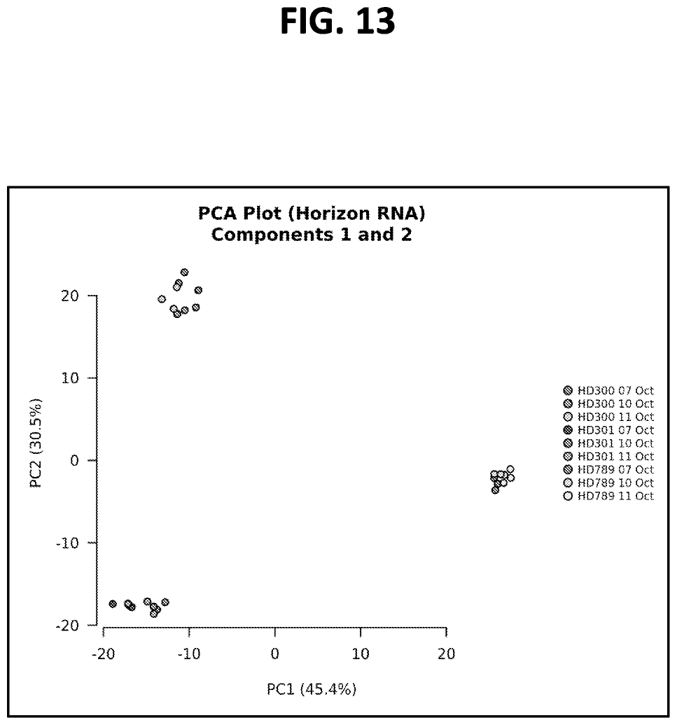

Repeatability and differential expression (RNA): Measurement of RNA expression using the disclosed methods is highly repeatable and reflective of biology. This is demonstrated by the principal component analysis (PCA) plot shown in FIG. 13, which was constructed using the RNA data from the nine replicates of samples HD300, HD301, and HD789. The first two principal components are graphed on the X and Y axes. The three different cell lines are strongly separated, demonstrating the expected differences in expression profiles and thus in their biology, but the replicates are tightly clustered together, demonstrating excellent repeatability between technical replicates and replicates run on different days.

The results of detecting of known mutations at known allelic frequencies using reference standards (DNA) are shown in FIG. 14. FIG. 14 shows a table of observed and expected allelic frequencies for each of the three reference standards and the three mixture samples. Each pair of sample and corresponding mixture/dilution sample are shown separately, to highlight the mutations carried by that sample. DNA variants in these samples include single nucleotide variants in EGFR (L861Q, L858R, T790M, G719S), KRAS (G12D, G13D, Q61H), and PIK3CA (E545), as well as a 15-base deletion variant (EGFR ΔE746-A750) and a 9-base insertion variant (EGFR V769_D770insASV). In all cases, the expected and observed allelic frequencies for these variants were well-correlated. Mutations were detected reliably at a range of frequencies, from 1% up, despite the small sample size of 1000 cells. Importantly, there were no false-positives signals detected; i.e., if a variant was not expected to be present in a sample, no significant counts for that mutation were detected.

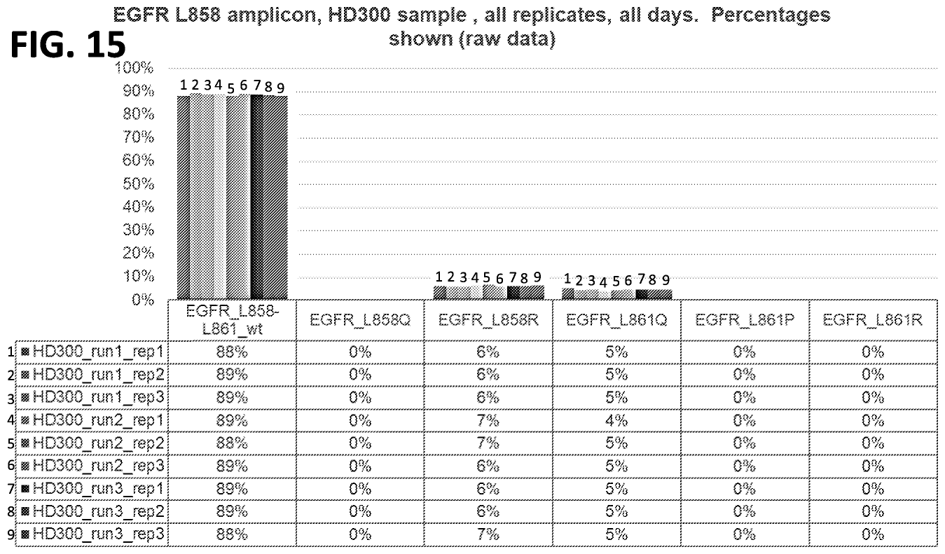

FIG. 15 displays the repeatability of individual measurements of DNA variants. A representative sample (HD300) and a representative amplicon (EGFR 858) are shown, with the percentages of wildtype and the indicated variants displayed. Each of the nine replicates is represented by a bar in the graph.

It is clear from these results that the DNA mutation status of these reference samples is faithfully and reliably measured using the disclosed methods. While mutations at a low allelic frequency (1% or less) were also detected in Example 3, the reference samples used in this Example are prepared and tested by an outside party and therefore represent an excellent calibration mark for the sensitivity of the described technique. Additionally, these standards included not only single nucleotide variants, but insertion and deletion variants, and provide an excellent example of the ability of the described techniques to detect multiple variations in a single sample, while simultaneously performing RNA profiling on the same sample.

Overall, the results indicate that the disclosed methods can both reliably measure the expression levels of multiple RNAs, as well as discern a range of single-base, insertion, and deletion changes at the DNA level, matching the expected results in reference samples, even when DNA mutations are present at 1% or less of the total allelic frequency for that locus.

In view of the many possible embodiments to which the principles of the disclosed invention may be applied, it should be recognized that the illustrated embodiments are only examples of the disclosure and should not be taken as limiting the scope of the disclosure. Rather, the scope of the invention is defined by the following claims. We therefore claim as our invention all that comes within the scope and spirit of these claims.

1. A method of determining a sequence of a target DNA molecule and a target RNA molecule in a sample, comprising:
lysing the sample with a lysis buffer, thereby generating a lysate comprising the target DNA molecule and the target RNA molecule;
amplifying the target DNA from a first portion of the lysate using at least one target DNA primer, thereby generating flanked amplicon regions (FARs);
incubating a second portion of the lysate with at least one nuclease protection probe comprising a flanking sequence (NPPF) under conditions sufficient for the NPPF to specifically bind to the target RNA molecule,
wherein the NPPF comprises:
a 5′-end and a 3′-end,
a sequence complementary to a region of the target RNA molecule, permitting specific binding between the NPPF and the target RNA molecule,
wherein the flanking sequence is located 5′, 3′, or both, to the sequence complementary to the target RNA molecule, wherein the 5′-flanking sequence is 5′ of the sequence complementary to the target RNA molecule, and the 3′-flanking sequence is 3′ of the sequence complementary to the target RNA molecule,
wherein the flanking sequence comprises at least 12 contiguous nucleotides not found in a nucleic acid molecule present in the sample,
if the NPPF comprises a 5′-flanking sequence, contacting the second portion of the lysate with a nucleic acid molecule comprising a sequence complementary to the 5′-flanking sequence (5CFS), under conditions sufficient for the 5′-flanking sequence to specifically hybridize to the 5CFS;
if the NPPF comprises a 3′-flanking sequence, contacting the second portion of the lysate with a nucleic acid molecule comprising a sequence complementary to the 3′-flanking sequence (3CFS) under conditions sufficient for the 3′-flanking sequence to specifically hybridize to the 3CFS;
generating an NPPF hybridized to the target RNA molecule, hybridized to the 3CFS, hybridized to the 5CFS, or hybridized to both the 3CFS and the 5CFS;
contacting the second portion of the lysate with a nuclease specific for single-stranded nucleic acid molecules under conditions sufficient to remove unbound nucleic acid molecules, thereby generating a digested second portion of the lysate comprising NPPF hybridized to the target RNA molecule, hybridized to the 3CFS, hybridized to the 5CFS, or hybridized to both the 3CFS and the 5CFS;
optionally separating the NPPF from the target RNA molecule and from the 3CFS, 5CFS, or both the 3CFS and the 5CFS, thereby generating a single stranded NPPF;
combining the FARs and the (i) single stranded NPPF or (ii) the NPPF hybridized to the target RNA molecule, hybridized to the 3CFS, hybridized to the 5CFS, or hybridized to both the 3CFS and the 5CFS, thereby generating a FARs:single stranded NPPF mixture;
amplifying the FARs and the single stranded NPPF in the FARs:single stranded NPPF mixture, thereby generating FAR amplicons and NPPF amplicons; and
sequencing at least a portion of the FAR amplicons and at least a portion of the NPPF amplicons, thereby determining the sequence of the target DNA molecule and the target RNA molecule in the sample.

2. The method of claim 1, wherein the NPPF comprises both a 5′-flanking sequence and a 3′-flanking sequence, and amplifying the FARs and the single stranded NPPF comprises contacting the FARs and the single stranded NPPF with a first amplification primer comprising a region that is identical to the 5′-flanking sequence and with a second amplification primer comprising a region that is complementary to the 3′-flanking sequence.

3. The method of claim 2, wherein the first amplification primer and/or the second amplification primer further comprises one or more sequences that permit attachment of an experimental tag, sequencing adaptor, or both, to the FAR amplicons or NPPF amplicons during the amplifying of the FARs and the single stranded NPPF.

4. The method of claim 3, wherein the experiment tag or sequencing adaptor is 12 to 50 nucleotides in length.

5. The method of claim 1, wherein the at least one target DNA primer comprises at least two target DNA primers, each comprising a flanking sequence at its 5′ end, wherein a first target DNA primer comprises a flanking sequence comprising a reverse-complement sequence of the 3′-flanking sequence, and wherein a second target DNA primer comprises a flanking sequence comprising the sequence of the 5′-flanking sequence.

6. The method of claim 1, wherein amplifying the target DNA from a first portion of the lysate comprises 8 to 12 amplification cycles.

7. The method of claim 1, wherein amplifying the amplifying the FARs and the single stranded NPPF comprises 8 to 25 amplification cycles.

8. The method of claim 1, wherein the target DNA molecule is a target genomic DNA molecule.

9. The method of claim 1, wherein the lysis buffer comprises a detergent and a chaotropic agent.

10. The method of claim 1, wherein the 5CFS and 3CFS are DNA.

11. The method of claim 1, wherein determining the sequence of the target RNA molecule in the sample comprises determining an absolute or relative abundance of the target RNA in the sample.

12. The method of claim 1, wherein the NPPF comprises a DNA molecule.

13. The method of claim 1, wherein the NPPF is 35 to 200 nucleotides in length.

14. The method of claim 1, wherein the sequence complementary to a region of the target nucleic acid molecule is 10 to 60 nucleotides in length.

15. The method of claim 1, wherein each flanking sequence is 12 to 50 nucleotides in length.

16. The method of claim 1, wherein the NPPF comprises a flanking sequence at the 5′-end and the 3′-end, and wherein the flanking sequence at the 5′-end differs from the flanking sequence at the 3′-end.

17. The method of claim 1, wherein the FARs are 100 to 200 nucleotides in length.

18. The method of claim 1, wherein the at least one target DNA primer comprises a Tm of 50° C. to 62° C., and the first and second amplification primers comprise a Tm of 50° C. to 62° C.

19. The method of claim 1, wherein the target RNA molecule is fixed, cross-linked, or insoluble.

20. The method of claim 1, wherein the sample is fixed.

21. The method of claim 1, wherein the sample is formalin fixed.

22. The method of claim 1, wherein the NPPF is a DNA, and the nuclease comprises an exonuclease, an endonuclease, or a combination thereof.

23. The method of claim 1, wherein the nuclease specific for single-stranded nucleic acid molecules comprises S1 nuclease.

24. The method of claim 1, wherein the method sequences or detects one or more target RNA molecules and one or more target DNA molecules in a plurality of samples simultaneously.

25. The method of claim 1, wherein the method sequences or detects at least two different target RNA molecules, and wherein the sample is contacted with at least two different NPPFs, each NPPF specific for a different target RNA molecule.

26. The method of claim 1, wherein the method sequences or detects at least two different target RNA molecules, and wherein the sample is contacted with at least one NPPF specific for the at least two different target RNA molecules.

27. The method of claim 1, wherein the method sequences or detects at least two different target DNA molecules, wherein the at least two different target DNA molecules comprise a wild type gene sequence and at least one mutation in the gene sequence.

28. The method of claim 1, wherein the method is performed on a plurality of samples and at least two different target RNA molecules and at least two different target DNA molecules are detected in each of the plurality of samples.

29. The method of claim 1, wherein at least one NPPF is specific for a miRNA target nucleic acid molecule and at least one NPPF is specific for an mRNA target nucleic acid molecule.

30. The method of claim 1, wherein the at least one NPPF comprises at least 10 different NPPFs.

31. The method of claim 1, wherein sequencing comprises next-generation sequencing or single molecule sequencing.

32. The method of claim 1, wherein determining the sequence of the at least one target DNA molecule determines if the target DNA molecule comprises a point mutation, insertions, and/or deletions, and determining the sequence of the at least one target RNA molecule determines abundance of the target RNA molecule.

33. The method of claim 2, further comprising
removing amplification primers after the amplifying the target DNA from a first portion of the lysate using at least one target DNA primer,
removing the first and second amplification primers after the amplifying of the FARs and the single stranded NPPF,
or both,
prior to the sequencing.

34. The method of claim 2, wherein the experiment tag comprises a nucleic acid sequence that permits identification of a sample, subject, treatment or target RNA or DNA molecule.

35. The method of claim 2, wherein the sequencing adaptor comprises a nucleic acid sequence that permits capture onto a sequencing platform.

36. The method of claim 2, wherein the experiment tag or sequencing adaptor is present on the 5′-end or 3′-end of the FAR amplicons and NPPF amplicons after amplifying the FARs and the single stranded NPPF.

37. The method of claim 1, further comprising:
comparing at least one NPPF amplicon sequence to a reference database, and determining a number of each of the identified at least one NPPF amplicons sequence; and/or
comparing at least one FAR amplicon sequence to a reference database, and determining any mutations in the at least one FAR amplicon sequence.

38. The method of claim 1, wherein the at least one target DNA primer comprises a phosphorotioate link between the last two bases at its 3′-end.

39. An isolated nucleic acid molecule comprising or consisting of the nucleic acid sequence of any one of SEQ ID NOS: 4-13 and 17-32.

40. A set of nucleic acid primers comprising:
SEQ ID NOs: 4 and 5;
SEQ ID NOs: 6 and 7;
SEQ ID NOs: 8 and 9;
SEQ ID NOs: 10 and 11;
SEQ ID NOs: 12 and 13;
SEQ ID NOs: 17 and 18;
SEQ ID NOs: 19 and 20;
SEQ ID NOs: 21 and 22;
SEQ ID NOs: 23 and 24;
SEQ ID NOs: 25 and 26;
SEQ ID NOs: 27 and 28;
SEQ ID NOs: 29 and 30;
SEQ ID NOs: 31 and 32; or
combinations thereof.


SEQUENCE LISTING (32 sequences)

SEQ ID NO: 1 - DNA, 25 bp, Artificial Sequence
  Exemplary 5′-flanking sequence
agttcagacgtgtgctcttccgatc

SEQ ID NO: 2 - DNA, 25 bp, Artificial Sequence
  Exemplary 3′- flanking sequence
gatcgtcggactgtagaactctgaa

SEQ ID NO: 3 - DNA, 25 bp, Artificial Sequence
  Exemplary forward primer
ttcagagttctacagtccgacgatc

SEQ ID NO: 4 - DNA, 53 bp, Artificial Sequence
  Exemplary BRAF primer
ttcagagttctacagtccgacgatccagtaaaaataggtgattttggtctagc

SEQ ID NO: 5 - DNA, 45 bp, Artificial Sequence
  Exemplary BRAF primer
agttcagacgtgtgctcttccgatcctgatgggacccactccatc

SEQ ID NO: 6 - DNA, 52 bp, Artificial Sequence
  Exemplary KRAS primer
ttcagagttctacagtccgacgatcaaatgactgaatataaacttgtggtag

SEQ ID NO: 7 - DNA, 47 bp, Artificial Sequence
  Exemplary KRAS primer
agttcagacgtgtgctcttccgatcaatgattctgaattagctgtat

SEQ ID NO: 8 - DNA, 44 bp, Artificial Sequence
  Exemplary EGFR primer
ttcagagttctacagtccgacgatcatctgcctcacctccaccg

SEQ ID NO: 9 - DNA, 42 bp, Artificial Sequence
  Exemplary EGFR primer
agttcagacgtgtgctcttccgatcgcagccgaagggcatga

SEQ ID NO: 10 - DNA, 50 bp, Artificial Sequence
  Exemplary EGFR primer
ttcagagttctacagtccgacgatctcttgaggatcttgaaggaaactga

SEQ ID NO: 11 - DNA, 45 bp, Artificial Sequence
  Exemplary EGFR primer
agttcagacgtgtgctcttccgatctatacaccgtgccgaacgca

SEQ ID NO: 12 - DNA, 64 bp, Artificial Sequence
  Exemplary first primer
  misc_feature (30)..(35): n is a, c, g, t or u
aatgatacggcgaccaccgagatctacacnnnnnncgacaggttcagagttctacagtccgacg

SEQ ID NO: 13 - DNA, 60 bp, Artificial Sequence
  Exemplary second primer
  misc_feature (25)..(30): n is a, c, g, t or u
caagcagaagacggcatacgagatnnnnnngtgactggagttcagacgtgtgctcttccg

SEQ ID NO: 14 - DNA, 50 bp, Artificial Sequence
  Exemplary BRAF sequence
ggtgattttggtctagctacagtgaaatctcgatggagtgggtcccatca

SEQ ID NO: 15 - DNA, 50 bp, Artificial Sequence
  Exemplary BRAF sequence
ggtgattttggtctagctacagagaaatctcgatggagtgggtcccatca

SEQ ID NO: 16 - DNA, 50 bp, Artificial Sequence
  Exemplary BRAF sequence
ggtgattttggtctagctacagaaaaatctcgatggagtgggtcccatca

SEQ ID NO: 17 - DNA, 44 bp, Artificial Sequence
  BRAF primer
ttcagagttctacagtccgacgatctgttcaaactgatgggacc

SEQ ID NO: 18 - DNA, 45 bp, Artificial Sequence
  BRAF primer
agttcagacgtgtgctcttccgatccatgaagacctcacagtaaa

SEQ ID NO: 19 - DNA, 45 bp, Artificial Sequence
  EGFR primer
ttcagagttctacagtccgacgatcccagggaccttaccttatac

SEQ ID NO: 20 - DNA, 45 bp, Artificial Sequence
  EGFR primer
agttcagacgtgtgctcttccgatcgctctcttgaggatcttgaa

SEQ ID NO: 21 - DNA, 44 bp, Artificial Sequence
  EGFR primer
ttcagagttctacagtccgacgatccacacagcaaagcagaaac

SEQ ID NO: 22 - DNA, 45 bp, Artificial Sequence
  EGFR primer
agttcagacgtgtgctcttccgatcccagaaggtgagaaagttaa

SEQ ID NO: 23 - DNA, 43 bp, Artificial Sequence
  EGFR primer
ttcagagttctacagtccgacgatccaggaagcctacgtgatg

SEQ ID NO: 24 - DNA, 41 bp, Artificial Sequence
  EGFR primer
agttcagacgtgtgctcttccgatcagccgaagggcatgag

SEQ ID NO: 25 - DNA, 41 bp, Artificial Sequence
  EGFR primer
ttcagagttctacagtccgacgatccaccgcagcatgtcaa

SEQ ID NO: 26 - DNA, 43 bp, Artificial Sequence
  EGFR primer
agttcagacgtgtgctcttccgatcacctaaagccacctcctt

SEQ ID NO: 27 - DNA, 46 bp, Artificial Sequence
  KRAS primer
ttcagagttctacagtccgacgatcattctgaattagctgtatcgt

SEQ ID NO: 28 - DNA, 48 bp, Artificial Sequence
  KRAS primer
agttcagacgtgtgctcttccgatcatgactgaatataaacttgtggt

SEQ ID NO: 29 - DNA, 47 bp, Artificial Sequence
  KRAS primer
ttcagagttctacagtccgacgatcgcaagtagtaattgatggagaa

SEQ ID NO: 30 - DNA, 45 bp, Artificial Sequence
  KRAS primer
agttcagacgtgtgctcttccgatcggcaaatacacaaagaaagc

SEQ ID NO: 31 - DNA, 46 bp, Artificial Sequence
  PIK3CA primer
ttcagagttctacagtccgacgatcaaagcaatttctacacgagat

SEQ ID NO: 32 - DNA, 45 bp, Artificial Sequence
  PIK3CA primer
agttcagacgtgtgctcttccgatcacttacctgtgactccatag